SEQUENCE LISTING (6 sequences)

SEQ ID NO: 1 - protein, 5 aa, Synthetic construct
HHHHH

SEQ ID NO: 2 - protein, 6 aa, Synthetic construct
HHHHHH

SEQ ID NO: 3 - protein, 10 aa, Synthetic construct
EQKLISEEDL

SEQ ID NO: 4 - protein, 8 aa, Synthetic construct
DYKDDDDK

SEQ ID NO: 5 - protein, 8 aa, Synthetic construct
WSHPQFEK

SEQ ID NO: 6 - protein, 9 aa, Synthetic construct
YPYDVPDYA

(19) United States
(12) Patent Application Publication
(10) Pub. No.: US 2023/0366045 A1
(43) Pub. Date: Nov. 16, 2023

(54) NUCLEIC ACID CROSSLINKING REAGENTS AND USES THEREOF

(71) Applicant: Promega Corporation, Madison, WI (US)

(72) Inventors: Subhanjan Mondal, Middleton, WI (US); Nathan Feirer, Marshall, WI (US); Min Zhou, Madison, WI (US); Michael Scurria, San Luis Obispo, CA (US); Wenhui Zhou, Madison, WI (US); Said Goueli, Fitchburg, WI (US); James J. Cali, Verona, WI (US)

(21) Appl. No.: 18/176,918

(22) Filed: Mar. 1, 2023

Related U.S. Application Data
(60) Provisional application No. 63/315,414, filed on Mar. 1, 2022.

(51) Int. Cl. C12Q 1/70; C12Q 1/6851; C07D 221/12; C07F 15/00; C07D 235/20; C07F 9/6558; C07D 417/06; C07D 417/14; C07D 495/04

(52) U.S. Cl. CPC C12Q 1/701; C12Q 1/6851; C07D 221/12; C07F 15/0093; C07D 235/20; C07F 9/65583; C07D 417/06; C07D 417/14; C07D 495/04; C12Q 1/6806

(57) ABSTRACT

Provided herein multifunctional compounds that bind and modify nucleic acids, forming crosslinks between the nucleic acid strands. In particular embodiments, the multifunctional nucleic-acid binding/modifying compounds are selective for nucleic acids of non-viable cells and viruses (i.e., they are excluded from or degraded within viable cells and viruses), and therefore find use in methods for detecting or discriminating viable and non-viable cells/viruses and nucleic acids therefrom.

### CROSS-REFERENCE TO RELATED APPLICATIONS

This application claims the benefit of U.S. Provisional Patent Application No. 63/315,414, filed on Mar. 1, 2022, which is incorporated by reference herein.

### SEQUENCE LISTING

The text of the computer readable sequence listing filed herewith, titled “40644-202_SEQUENCE_LISTING”, created Aug. 3, 2023, having a file size of 6,223 bytes, is hereby incorporated by reference in its entirety.

### FIELD

Provided herein are multifunctional compounds that bind and/or modify nucleic acids by forming crosslinks between the nucleic acid strands. In particular embodiments, the multifunctional nucleic acid binding/modifying compounds are selective for nucleic acids of non-viable cells and viruses (i.e., they are excluded from or degraded within viable cells and viruses), and therefore find use in methods for detecting or discriminating viable and non-viable cells/viruses and nucleic acids therefrom.

### BACKGROUND

Nucleic acid-based analytical methods, ranging from species-specific PCR to metagenomics, have greatly expanded our understanding of microbiological diversity in natural samples. However, cell viability cannot easily be assessed by standard DNA-targeted methods such as PCR or qPCR and isothermal amplification methods.

Propidium monoazide (PMA) has been shown to differentiate DNA associated with viable cells from DNA associated with non-viable cells in a technique known as viability PCR (vPCR). In a sample having both live and dead cells, PMA can access the DNA from the dead cells owing to the compromised cell membrane. PMA has a photoreactive azide moiety that covalently modifies DNA upon photolysis, rendering the DNA unable to serve as a template in an amplification reaction. After the photoactivation step, lysis of the remaining viable cells exposes their DNA to allow for amplification. However, variations in light spectrum and intensity can result in sample-to-sample variability, particularly in complex, turbid samples. These difficulties limit the broad utilization and adoption of the vPCR technique.

### SUMMARY

Provided herein are multifunctional compounds that bind and modify nucleic acids by forming crosslinks between the nucleic acid strands. In particular embodiments, the multifunctional nucleic acid binding/modifying compounds are selective for nucleic acids of non-viable cells and viruses (i.e., they are excluded from or degraded within viable cells and viruses), and therefore find use in methods for detecting or discriminating viable and non-viable cells/viruses and nucleic acids therefrom.

In some embodiments, provided herein are multifunctional compounds, or a salt thereof, the multifunctional compound comprising: (A) a RNA binding moiety (“RAB moiety”); (B) a live/dead cell differentiating moiety (“LDCD moiety”); and (C) a nucleic acid modifying moiety (“NAM moiety”). In some embodiments, the multifunctional compound has the structure:

A-B—C,

wherein A is the RAB moiety, B is the LDCD moiety, C is the nucleic acid modifying moiety. In some embodiments, the multifunctional compound has more than one RAB moiety, more than one LDCD moiety, and/or more than one NAM moiety.

In some embodiments, an RAB moiety comprises structure of formula (I):

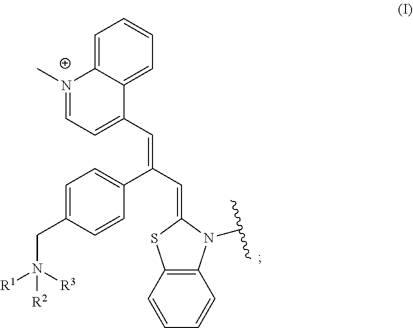

or a tautomer or a salt thereof, wherein:

R¹ and R² are each independently selected from hydrogen, C₁-C₆ alkyl, C₂-C₆ alkenyl, C₂-C₆ alkynyl, aryl, and aryl-C₁-C₆ alkyl, each of which is optionally substituted with 1-3 substituents, or R¹ and R² are taken together with the nitrogen atom to which they are attached to form an optionally substituted heterocyclyl or heteroaryl ring; and

R³ is absent or is C₁-C₆ alkyl.

In some embodiments, R¹ and R² are each independently selected from C₁-C₆ alkyl, and R³ is absent or is C₁-C₆ alkyl.

In some embodiments, R¹, R², and R³ are each independently selected from C₁-C₆ alkyl.

In some embodiments, R¹ and R² are each independently selected from C₁-C₆ alkyl (e.g., methyl or ethyl), and R³ is absent.

In some embodiments, R³ is absent, and R¹ and R² are taken together with the nitrogen atom to which they are attached to form an optionally substituted monocyclic 5- or 6-membered heterocyclyl or heteroaryl ring having 1, 2, or 3 heteroatoms independently selected from N, O, and S.

In some embodiments, R³ is absent, and R¹ and R² are taken together with the nitrogen atom to which they are attached to form a pyrrolidinyl, piperidinyl, morpholino, piperazinyl, or imidazolyl ring, each of which is optionally substituted with one substituent (e.g., C₁-C₆ alkyl, such as methyl).

In some embodiments, the group —NR¹R²R³ in the moiety of formula (I) has a formula selected from:

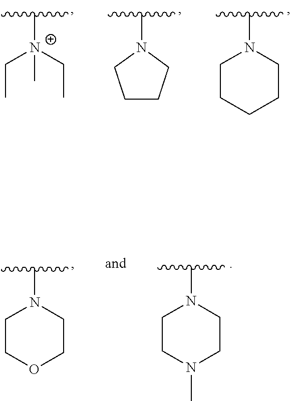

In some embodiments, the RAB moiety has a structure selected from:

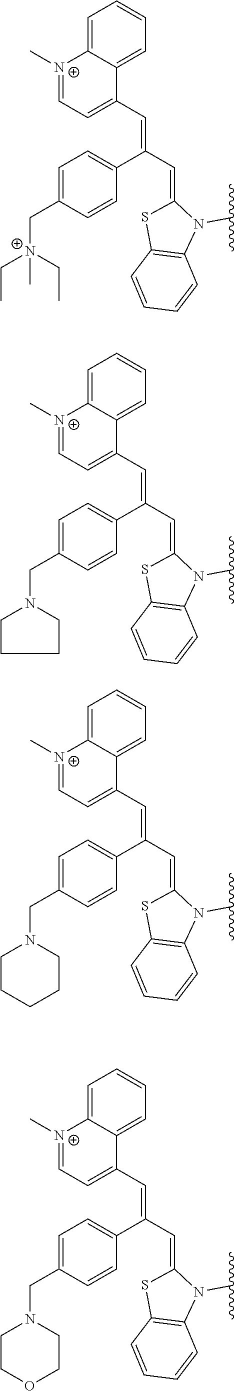 [SMILES: *N1/C(=C/C(=C/c2cc[n+](C)c3ccccc23)c2ccc(C[N+](C)(CC)CC)cc2)Sc2ccccc21 ; *N1/C(=C/C(=C/c2cc[n+](C)c3ccccc23)c2ccc(CN3CCCC3)cc2)Sc2ccccc21 ; *N1/C(=C/C(=C/c2cc[n+](C)c3ccccc23)c2ccc(CN3CCCCC3)cc2)Sc2ccccc21 ; *N1/C(=C/C(=C/c2cc[n+](C)c3ccccc23)c2ccc(CN3CCOCC3)cc2)Sc2ccccc21]

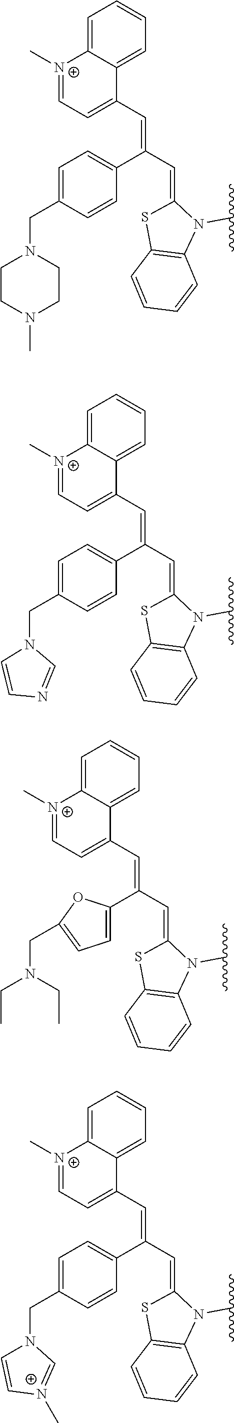 [SMILES: *N1/C(=C/C(=C/c2cc[n+](C)c3ccccc23)c2ccc(CN3CCN(C)CC3)cc2)Sc2ccccc21 ; *N1/C(=C/C(=C/c2cc[n+](C)c3ccccc23)c2ccc(Cn3ccnc3)cc2)Sc2ccccc21 ; *N1/C(=C/C(=C/c2cc[n+](C)c3ccccc23)c2ccc(CN(CC)CC)o2)Sc2ccccc21 ; *N1/C(=C/C(=C/c2cc[n+](C)c3ccccc23)c2ccc(Cn3cc[n+](C)c3)cc2)Sc2ccccc21]

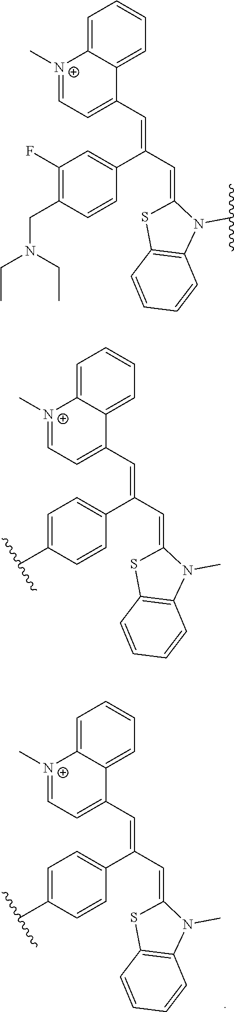

In some embodiments, the NAM moiety comprises a bischloroethylamine (nitrogen mustard) moiety, a platinum-based moiety, a 1-(chloromethyl)-2,3-dihydro-1H-benzo[e]indolyl moiety, or a pyrrolo[2,1-c][1,4]benzodiazepine (PBD) moiety. In some embodiments, the NAM moiety has a structure selected from:

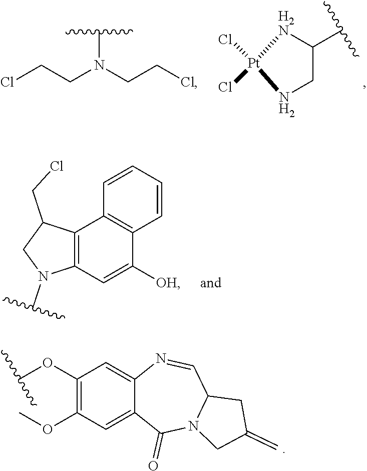

In some embodiments, the LDCD moiety comprises at least one charged moiety. In some embodiments, the LDCD moiety comprises at least one quaternary ammonium group. In some embodiments, the LDCD moiety comprises at least one poly(ethylene glycol) moiety. In some embodiments, the poly(ethylene glycol) moiety has formula: —(CH₂CH₂O)ₙ—, wherein n is 2, 3, 4, 5, 6, 7, 8, 9, or 10. In some embodiments, the LDCD moiety comprises a functional group bound to a solid support. In some embodiments, the LDCD moiety comprises a metabolically cleavable group.

In some embodiments, the multifunctional compound is selected from:

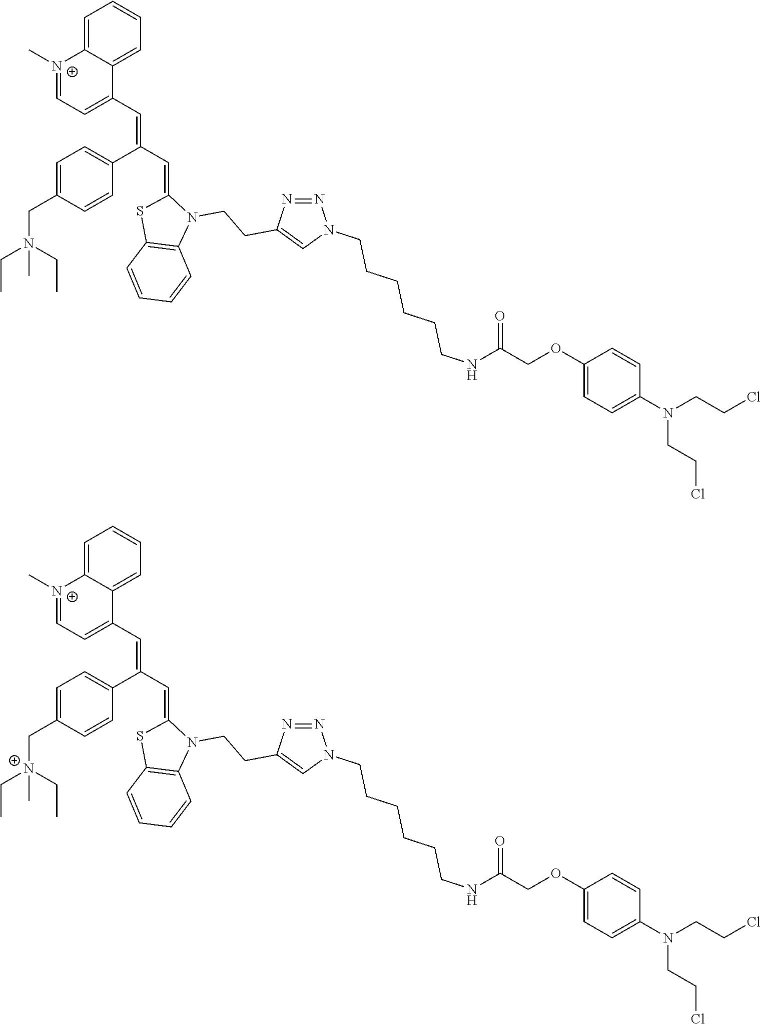

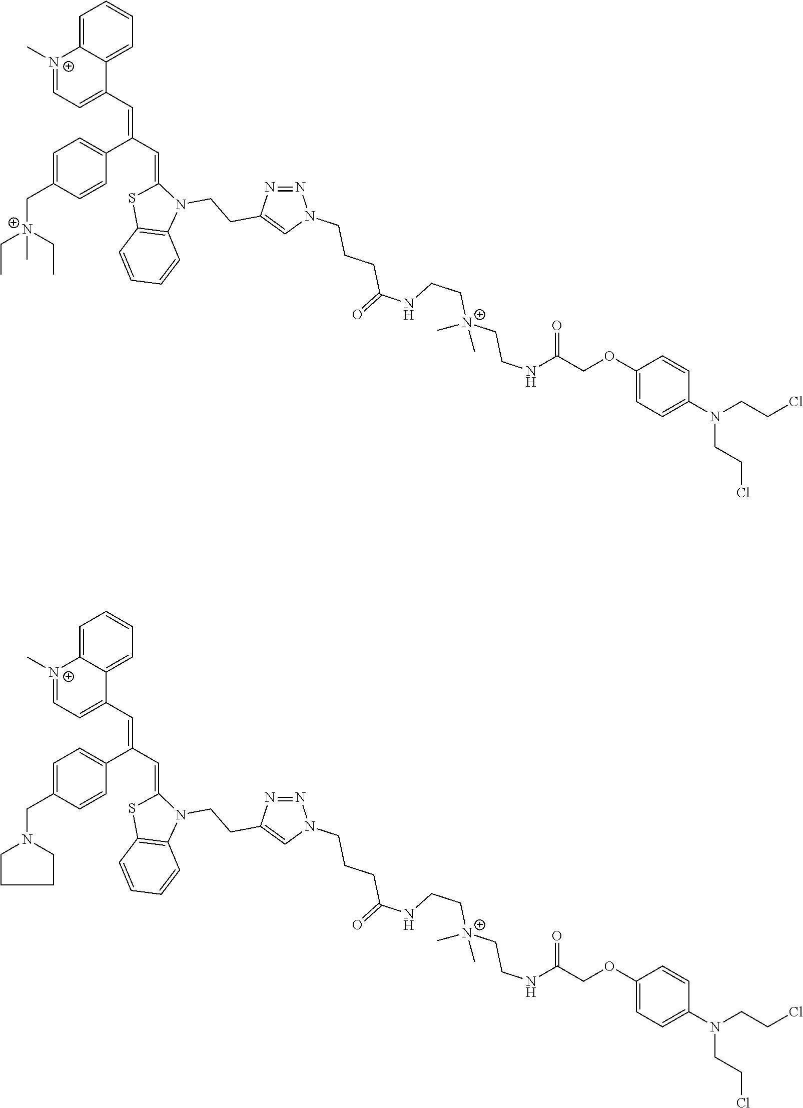 [SMILES: CC[N+](C)(CC)Cc1ccc(C(=C\c2cc[n+](C)c3ccccc23)/C=C2\Sc3ccccc3N2CCc2cn(CCCC(=O)NCC[N+](C)(C)CCNC(=O)COc3ccc(N(CCCl)CCCl)cc3)nn2)cc1 ; C[n+]1ccc(/C=C(/C=C2\Sc3ccccc3N2CCc2cn(CCCC(=O)NCC[N+](C)(C)CCNC(=O)COc3ccc(N(CCCl)CCCl)cc3)nn2)c2ccc(CN3CCCC3)cc2)c2ccccc21]

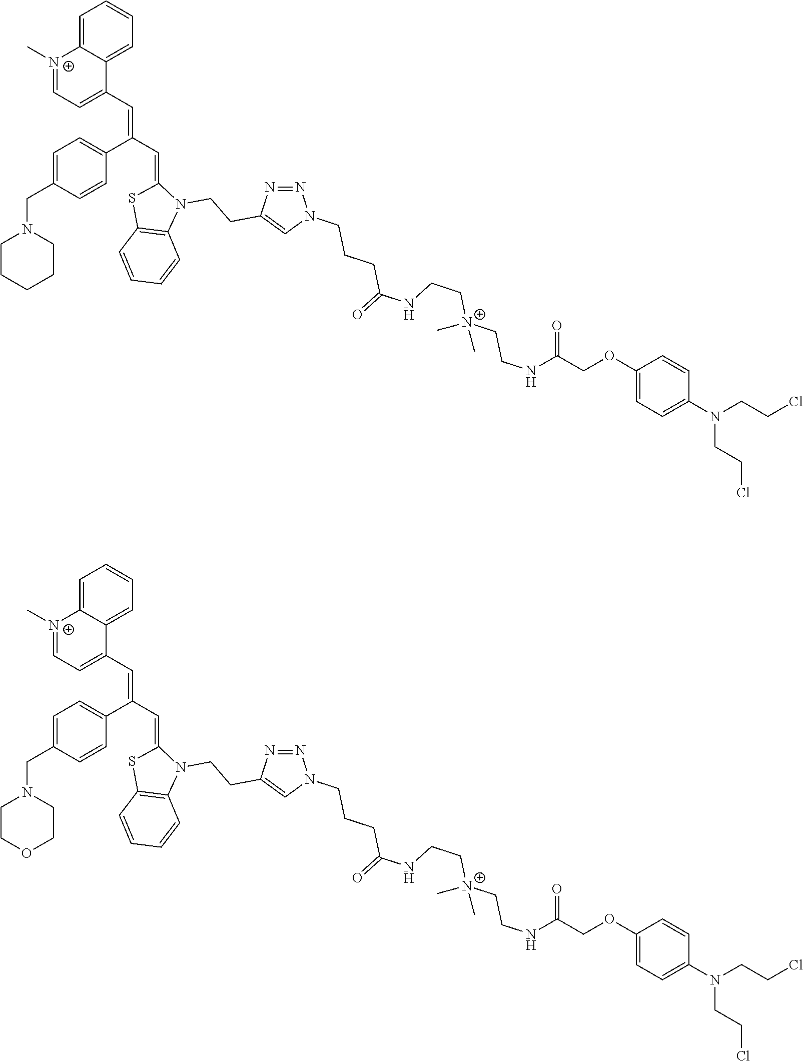 [SMILES: C[n+]1ccc(/C=C(/C=C2\Sc3ccccc3N2CCc2cn(CCCC(=O)NCC[N+](C)(C)CCNC(=O)COc3ccc(N(CCCl)CCCl)cc3)nn2)c2ccc(CN3CCCCC3)cc2)c2ccccc21 ; C[n+]1ccc(/C=C(/C=C2\Sc3ccccc3N2CCc2cn(CCCC(=O)NCC[N+](C)(C)CCNC(=O)COc3ccc(N(CCCl)CCCl)cc3)nn2)c2ccc(CN3CCOCC3)cc2)c2ccccc21]

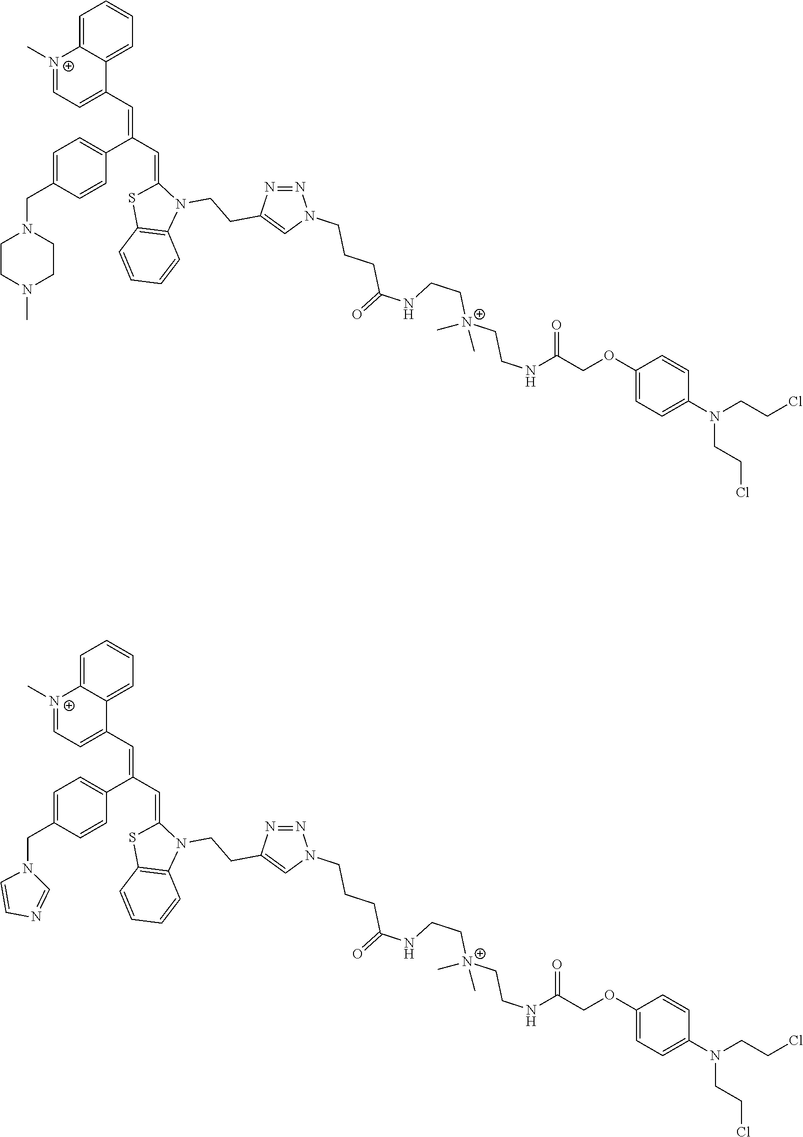 [SMILES: CN1CCN(Cc2ccc(C(=C\c3cc[n+](C)c4ccccc34)/C=C3\Sc4ccccc4N3CCc3cn(CCCC(=O)NCC[N+](C)(C)CCNC(=O)COc4ccc(N(CCCl)CCCl)cc4)nn3)cc2)CC1 ; C[n+]1ccc(/C=C(/C=C2\Sc3ccccc3N2CCc2cn(CCCC(=O)NCC[N+](C)(C)CCNC(=O)COc3ccc(N(CCCl)CCCl)cc3)nn2)c2ccc(Cn3ccnc3)cc2)c2ccccc21]

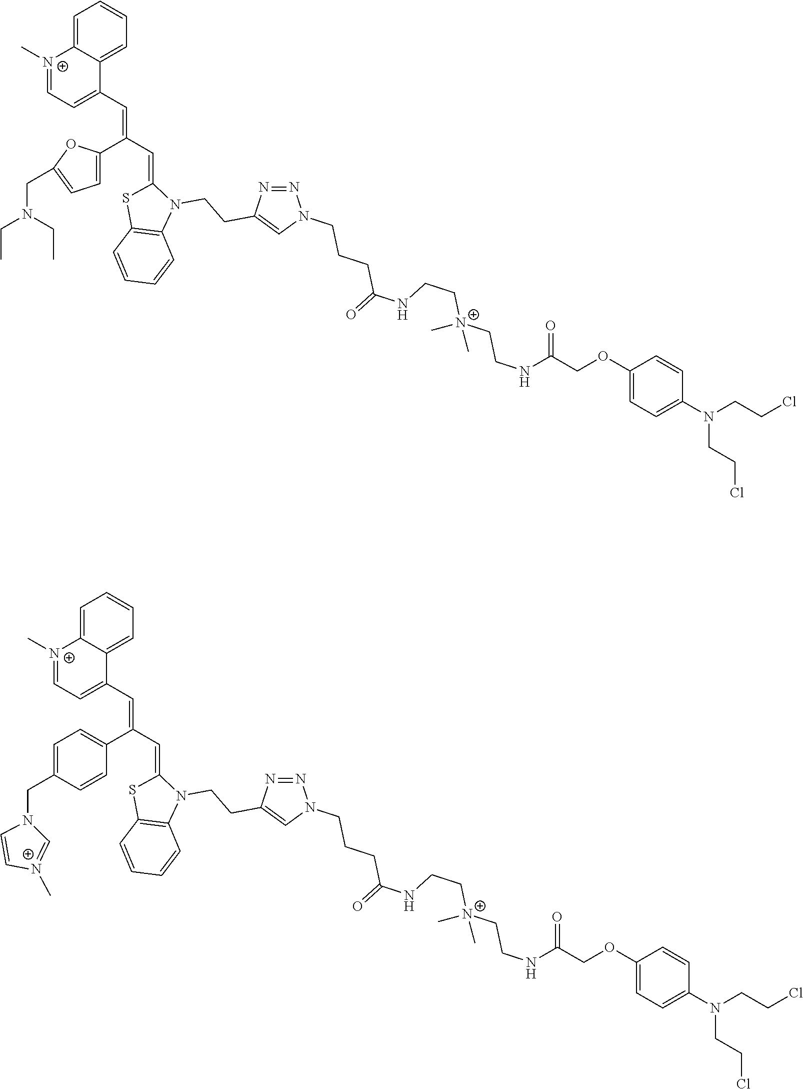 [SMILES: CCN(CC)Cc1ccc(C(=C\c2cc[n+](C)c3ccccc23)/C=C2\Sc3ccccc3N2CCc2cn(CCCC(=O)NCC[N+](C)(C)CCNC(=O)COc3ccc(N(CCCl)CCCl)cc3)nn2)o1 ; C[n+]1ccn(Cc2ccc(C(=C\c3cc[n+](C)c4ccccc34)/C=C3\Sc4ccccc4N3CCc3cn(CCCC(=O)NCC[N+](C)(C)CCNC(=O)COc4ccc(N(CCCl)CCCl)cc4)nn3)cc2)c1]

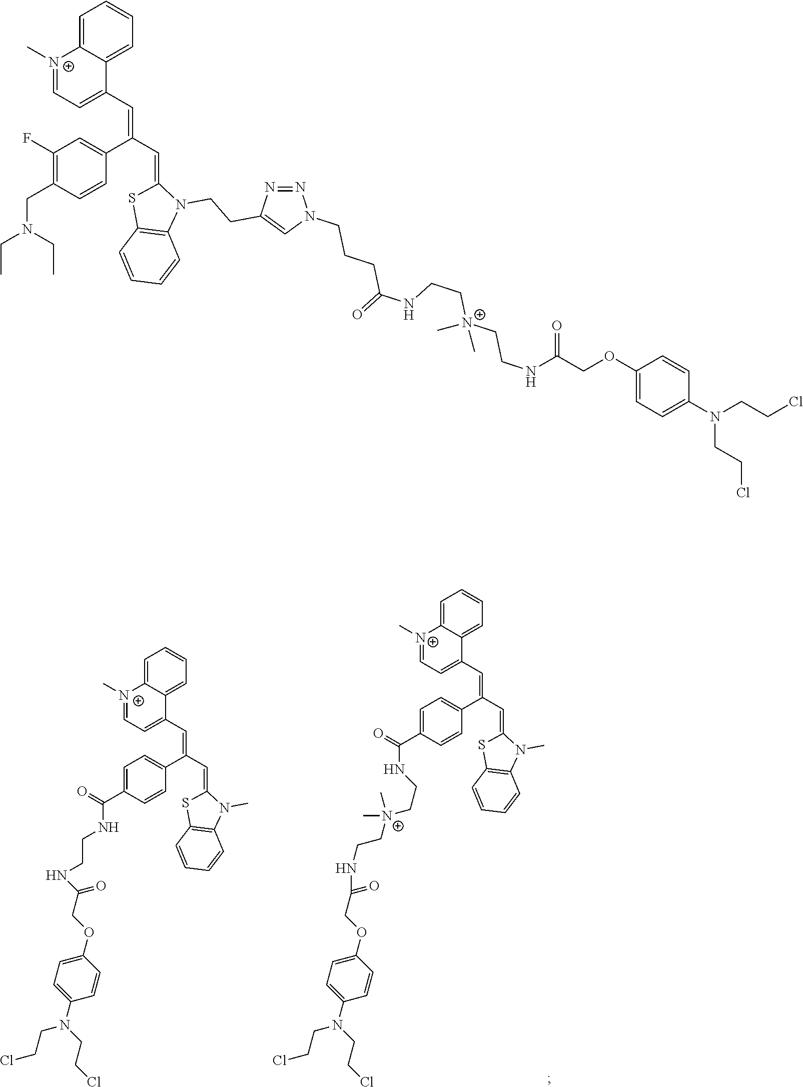 [SMILES: CCN(CC)Cc1ccc(C(=C\c2cc[n+](C)c3ccccc23)/C=C2\Sc3ccccc3N2CCc2cn(CCCC(=O)NCC[N+](C)(C)CCNC(=O)COc3ccc(N(CCCl)CCCl)cc3)nn2)cc1F ; CN1/C(=C/C(=C/c2cc[n+](C)c3ccccc23)c2ccc(C(=O)NCCNC(=O)COc3ccc(N(CCCl)CCCl)cc3)cc2)Sc2ccccc21 ; CN1/C(=C/C(=C/c2cc[n+](C)c3ccccc23)c2ccc(C(=O)NCC[N+](C)(C)CCNC(=O)COc3ccc(N(CCCl)CCCl)cc3)cc2)Sc2ccccc21]

and a salt of any thereof.

In some embodiments, provided herein are methods of detecting a viable microorganism or cell in a sample, the method comprising: (a) contacting the sample with a compound of any one of claims 1-20, or a salt thereof, to form a first mixture; (b) contacting the first mixture with an inactivating agent to form a second mixture; and (c) amplifying nucleic acids from the second mixture to produce a detectable signal, wherein the signal is indicative of the presence of a viable microorganism or cell in the sample. In some embodiments, the method does not include a photoactivation step. In some embodiments, the method does not include a culturing step. In some embodiments, step (a) comprises contacting the sample with the multifunctional compound or the salt thereof for 5 minutes to 180 minutes. In some embodiments, step (a) comprises contacting the sample with the multifunctional compound or the salt thereof for 60 minutes to 120 minutes. In some embodiments, step (a) comprises adding to the sample a composition comprising the multifunctional compound, or a salt thereof, in a solvent. In some embodiments, the solvent is dimethylsulfoxide. In some embodiments, in step (a), the first mixture comprises the multifunctional compound at a concentration of 5-100 micromolar. In some embodiments, the inactivating agent comprises a nucleophile selected from an amine and a thiol. In some embodiments, the inactivating agent is selected from cysteine, glutathione, a dNTP, guanine, an amine-containing buffer, and a mixture of any thereof. In some embodiments, step (c) comprises: (i) lysing cells in the second mixture to form a lysed sample; (ii) adding a reverse transcriptase, DNA polymerase, and amplification reagents to the lysed sample to form a mixture; and (iii) subjecting the mixture to a thermal cycling protocol to amplify the nucleic acid from the sample. In some embodiments, methods further comprise a step of removing contaminants and/or cellular debris from the lysed sample, prior to adding the reverse transcriptase, DNA polymerase, and amplification reagents. In some embodiments, the amplification reagents comprise at least one primer, deoxynucleotide triphosphates, a buffer, and a magnesium salt. In some embodiments, the amplification reagents comprise forward and reverse primers for a target amplicon in the sample. In some embodiments, the detectable signal is a fluorescent signal. In some embodiments, the microorganism is an RNA virus. In some embodiments, the RNA virus is of the order Nidovirales, Picornavirales, or Tymovirales. In some embodiments, the RNA virus is of the family Arteriviridae, Coronaviridae, Mesoniviridae, Roniviridae, Dicistroviridae, Iflaviridae, Marnaviridae, Picornaviridae, Secoviridae, Alphaflexiviridae, Betaflexiviridae, Gammaflexiviridae, Tymoviridae, Caliciviridae, Flaviviridae, or Togaviridae. In some embodiments, the RNA virus is a human pathogen. In some embodiments, the RNA virus is selected from SARS-CoV-2, HIV-1 Rhinovirus, Hepatitis A virus, Yellow fever virus, West Nile virus, Hepatitis C virus, Dengue fever virus, and Zika virus.

In some embodiments, provided herein are methods of removing RNA from a sample, the method comprising contacting the sample with a multifunctional compound comprising a RAB described herein. In some embodiments, the multifunctional compound is immobilized on a solid support.

In some embodiments, provided herein are systems or kits comprising a multifunctional compound comprising a RAB described herein. In some embodiments, systems or kits further comprise a reverse transcriptase and a DNA polymerase. In some embodiments, systems or kits further comprise one or more amplification reagents. In some embodiments, systems or kits further comprise forward and reverse primers for a target nucleic acid sequence of an RNA virus. In some embodiments, the RNA virus is of the order Nidovirales, Picornavirales, or Tymovirales. In some embodiments, the RNA virus is of the family Arteriviridae, Coronaviridae, Mesoniviridae, Roniviridae, Dicistroviridae, Iflaviridae, Marnaviridae, Picornaviridae, Secoviridae, Alphaflexiviridae, Betaflexiviridae, Gammaflexiviridae, Tymoviridae, Caliciviridae, Flaviviridae, or Togaviridae. In some embodiments, the RNA virus is a human pathogen. In some embodiments, the RNA virus is selected from SARS-CoV-2, HIV-1 Rhinovirus, Hepatitis A virus, Yellow fever virus, West Nile virus, Hepatitis C virus, Dengue fever virus, and Zika virus.

In some embodiments, provided herein are multifunctional compounds or a salt thereof, the multifunctional compound comprising: (A) a nucleic acid binding moiety (“NAB moiety”); (B) a live/dead cell differentiating moiety (“LDCD moiety”); (C) a nucleic acid modifying moiety (“NAM moiety”); and (D) an affinity or conjugation moiety. In some embodiments, the multifunctional compound has the structure: A-B(D)-C, wherein A is the NAB moiety, B is the LDCD moiety, C is the nucleic acid modifying moiety, and D is the affinity or conjugation moiety. In some embodiments, the multifunctional compound has more than one NAB moiety, more than one LDCD moiety, and/or more than one NAM moiety. In some embodiments, the NAB is a RNA binding moiety (“RAB moiety”).

In some embodiments, the RAB moiety comprises structure of formula (I):

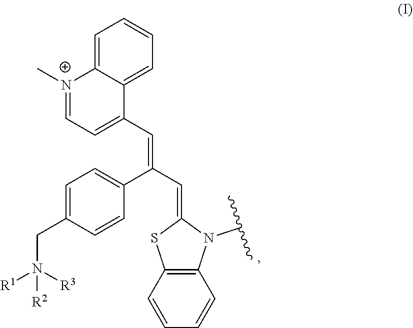

or a tautomer or a salt thereof, wherein R¹ and R² are each independently selected from hydrogen, C₁-C₆ alkyl, C₂-C₆ alkenyl, C₂-C₆ alkynyl, aryl, and aryl-C₁-C₆ alkyl, each of which is optionally substituted with 1-3 substituents, or R¹ and R² are taken together with the nitrogen atom to which they are attached to form an optionally substituted heterocyclyl or heteroaryl ring; and R³ is absent or is C₁-C₆ alkyl. In some embodiments, R¹ and R² are each independently selected from C₁-C₆ alkyl, and R³ is absent or is C₁-C₆ alkyl. In some embodiments, R¹, R², and R³ are each independently selected from C₁-C₆ alkyl. In some embodiments, R¹ and R² are each independently selected from C₁-C₆ alkyl (e.g., methyl or ethyl), and R³ is absent. In some embodiments, R³ is absent, and R¹ and R² are taken together with the nitrogen atom to which they are attached to form an optionally substituted monocyclic 5- or 6-membered heterocyclyl or heteroaryl ring having 1, 2, or 3 heteroatoms independently selected from N, O, and S. In some embodiments, R³ is absent, and R¹ and R² are taken together with the nitrogen atom to which they are attached to form a pyrrolidinyl, piperidinyl, morpholino, piperazinyl, or imidazolyl ring, each of which is optionally substituted with one substituent (e.g., C₁-C₆ alkyl, such as methyl). In some embodiments, the group —NR¹R²R³ in the moiety of formula (I) has a formula selected from:

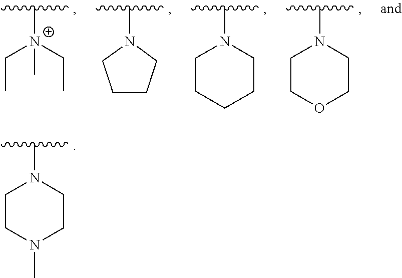

In some embodiments, the RAB moiety has a structure selected from:

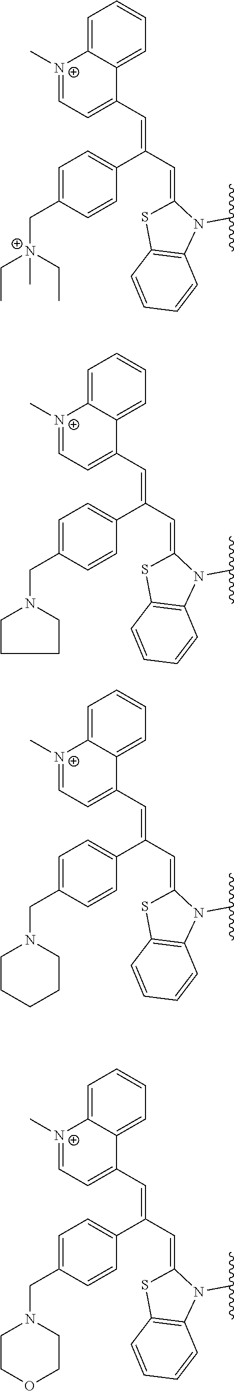 [SMILES: *N1/C(=C/C(=C/c2cc[n+](C)c3ccccc23)c2ccc(C[N+](C)(CC)CC)cc2)Sc2ccccc21 ; *N1/C(=C/C(=C/c2cc[n+](C)c3ccccc23)c2ccc(CN3CCCC3)cc2)Sc2ccccc21 ; *N1/C(=C/C(=C/c2cc[n+](C)c3ccccc23)c2ccc(CN3CCCCC3)cc2)Sc2ccccc21 ; *N1/C(=C/C(=C/c2cc[n+](C)c3ccccc23)c2ccc(CN3CCOCC3)cc2)Sc2ccccc21]

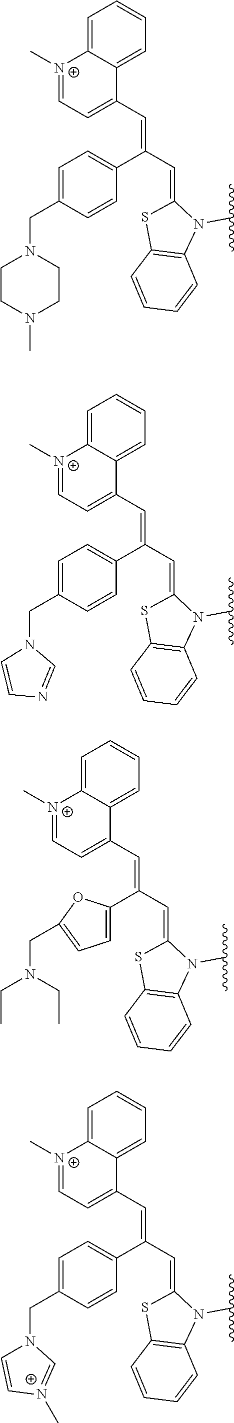 [SMILES: *N1/C(=C/C(=C/c2cc[n+](C)c3ccccc23)c2ccc(CN3CCN(C)CC3)cc2)Sc2ccccc21 ; *N1/C(=C/C(=C/c2cc[n+](C)c3ccccc23)c2ccc(Cn3ccnc3)cc2)Sc2ccccc21 ; *N1/C(=C/C(=C/c2cc[n+](C)c3ccccc23)c2ccc(CN(CC)CC)o2)Sc2ccccc21 ; *N1/C(=C/C(=C/c2cc[n+](C)c3ccccc23)c2ccc(Cn3cc[n+](C)c3)cc2)Sc2ccccc21]

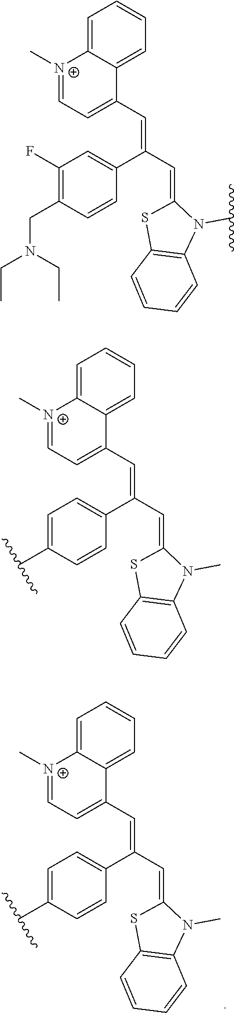

In some embodiments, the NAB is a DNA binding moiety (“DAB moiety”). In some embodiments, the DAB moiety is a groove-binding moiety, an intercalating moiety, or a mixed-mode binding moiety. In some embodiments, the DAB moiety comprises a bibenzimidazole moiety or a phenylphenanthridium moiety. In some embodiments, the DAB moiety has a structure selected from:

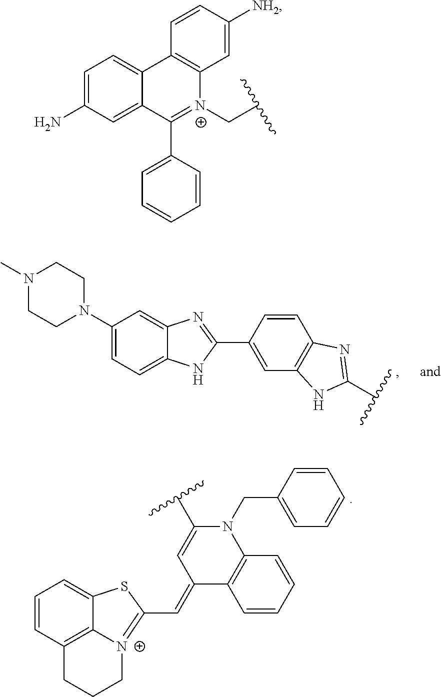 [SMILES: *C[n+]1c(-c2ccccc2)c2cc(N)ccc2c2ccc(N)cc21 ; *c1nc2ccc(-c3nc4cc(N5CCN(C)CC5)ccc4[nH]3)cc2[nH]1 ; *C1=C/C(=C\c2sc3cccc4c3[n+]2CCC4)c2ccccc2N1Cc1ccccc1]

In some embodiments, the NAM moiety comprises a bischloroethylamine (nitrogen mustard) moiety, a platinum-based moiety, a 1-(chloromethyl)-2,3-dihydro-1H-benzo[e]indolyl moiety, or a pyrrolo[2,1-c][1,4]benzodiazepine (PBD) moiety. In some embodiments, the NAM moiety has a structure selected from:

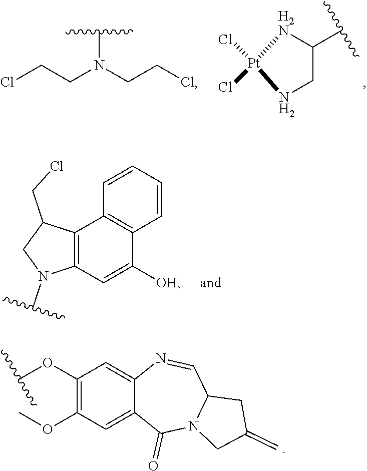

In some embodiments, the LDCD moiety comprises at least one charged moiety. In some embodiments, the LDCD moiety comprises at least one quaternary ammonium group. In some embodiments, the LDCD moiety comprises at least one poly(ethylene glycol) moiety. In some embodiments, the poly(ethylene glycol) moiety has formula: —(CH₂CH₂O)ₙ—, wherein n is 2, 3, 4, 5, 6, 7, 8, 9, or 10. In some embodiments, the LDCD moiety comprises one or more of the following groups:

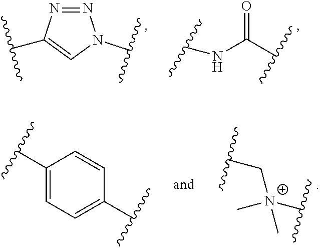

In some embodiments, the LDCD moiety further comprises one or more alkylene groups (—(CH₂)ₙ—), ether groups (—O—), thioether groups (—S—), ester linkages (—C(O)O—), carbamate linkages (—OC(O)NH—), sulfonamide linkages (—S(O)₂NH—), and any combination thereof. In some embodiments, the LDCD moiety is a branched LDCD moiety. In some embodiments, the branched LDCD moiety comprises one of the following branch-point functional groups:

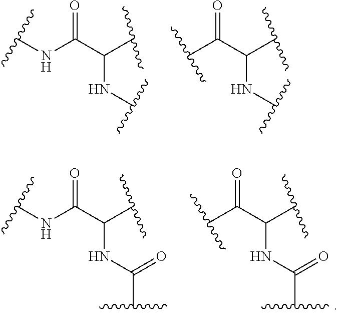 [SMILES: *NC(=O)C(*)N* ; *NC(*)C(*)=O ; *NC(=O)C(*)NC(*)=O ; *C(=O)NC(*)C(*)=O]

In some embodiments, the branched LDCD moiety comprises:

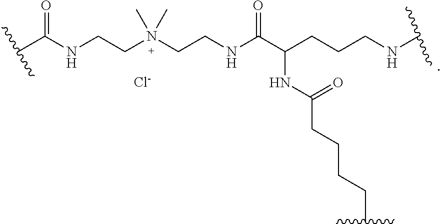

In some embodiments, the multifunctional compound comprises an affinity moiety. In some embodiments, the affinity moiety is selected from biotin, a peptide tag, and an epitope. In some embodiments, the multifunctional compound comprises the structure of:

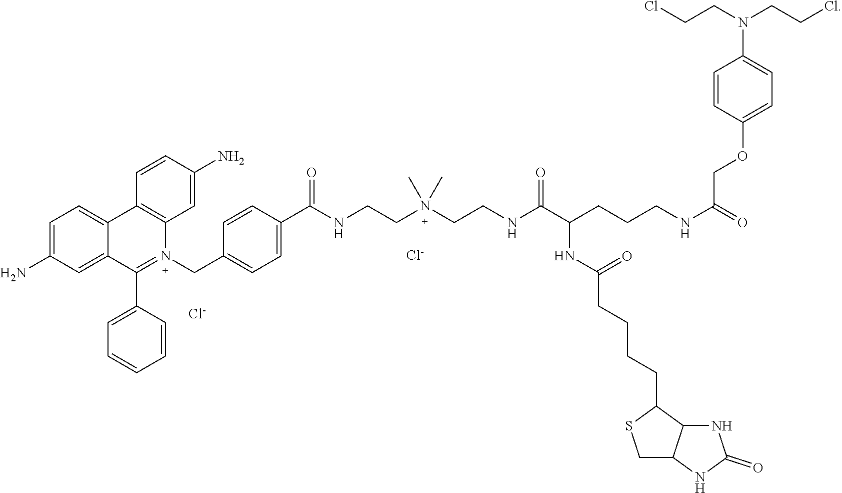

In some embodiments, the multifunctional compound comprises a conjugation moiety. In some embodiments, the conjugation moiety comprises a haloalkane. In some embodiments, the haloalkane is of the structure —(CH₂)ₙ—Y, wherein n is 2, 3, 4, 5, 6, 7, 8, 9, 10, 11, or 12, and Y is F, Cl, Br, or I. In some embodiments, n is 6 and Y is Cl.

In some embodiments, provided herein are systems or kits comprising a multifunctional compound herein comprising an affinity moiety or conjugation moiety. In some embodiments, the system or kit further comprises a reverse transcriptase and/or DNA polymerase, and one or more amplification reagents and/or primers. In some embodiments, the system or kit further comprises an affinity agent or a conjugation agent.

In some embodiments, compounds, methods, systems, kits, etc. described in U.S. Ser. No. 17/464,476 (incorporated by reference in its entirety) find use in embodiments herein.

### BRIEF DESCRIPTION OF THE DRAWINGS

FIG. 1 shows a representative live-dead differentiation assay, which does not require a photoactivation step.

FIG. 2 shows data from vPCR reactions conducted in live and dead cells with several concentrations of compound CS0775.

FIG. 3 shows data from vPCR reactions performed in a representative Gram-negative bacterium in the presence of compound CS0775 in varying concentrations of DMSO and two amplicon sizes.

FIG. 4 shows data from vPCR reactions in representative bacterial strains in the presence of compound CS0775 using primers optimized for various amplicon sizes.

FIG. 5 shows data from vPCR reactions in representative bacterial strains in the presence of varying concentrations of compound CS0775.

FIG. 6 shows data from vPCR reactions in representative bacterial strains in the presence of several compounds of the disclosure at varying concentrations.

FIG. 7 shows data from vPCR reactions in samples containing various cell concentrations in the presence of compound CS0775.

FIG. 8 shows data from vPCR reactions in samples with various proportional combinations of live and dead cells in the presence of compound CS0775.

FIG. 9 shows data from vPCR reactions conducted with intact and heat-inactivated virus particles treated with compound CS0775.

FIG. 10 shows an exemplary viral capsid integrity assay using multifunctional nucleic acid crosslinkers herein.

FIG. 11 shows ΔCt values for different treatment conditions and amplicon length in a SARS-CoV-2 Viral infectivity assay.

FIG. 12A-C shows results of a AAV capsid integrity assay.

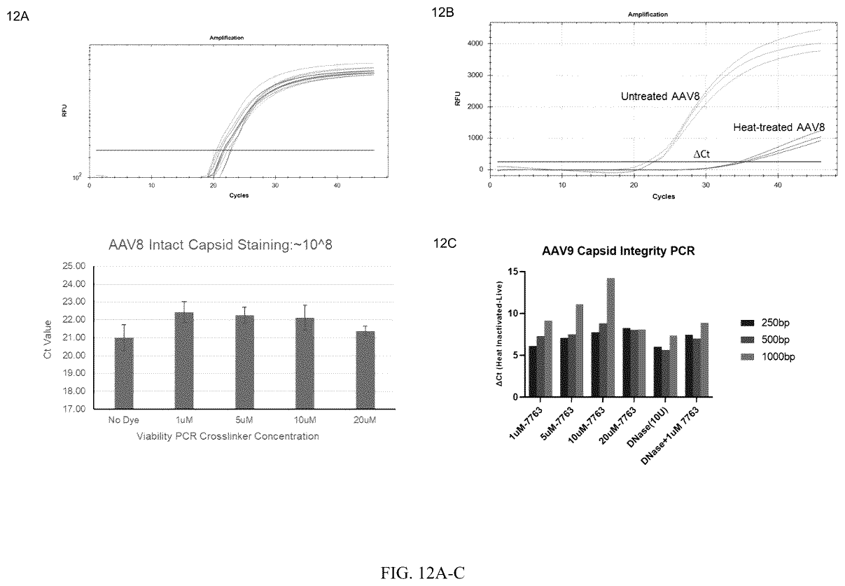

### DETAILED DESCRIPTION

Provided herein are multifunctional compounds that bind and modify nucleic acids by forming crosslinks between the nucleic acid strands. In particular embodiments, the multifunctional nucleic acid binding/modifying compounds are selective for nucleic acids of non-viable cells and viruses (i.e., they are excluded from or degraded within viable cells and viruses), and therefore find use in methods for detecting or discriminating viable and non-viable cells/viruses and nucleic acids therefrom.

The multifunctional compounds herein comprise a nucleic acid binding moiety (e.g., DNA-selective binding moiety, RNA-selective binding moiety, non-selective nucleic acid binding moiety, etc.), a nucleic acid modifying moiety, and a viability differentiation motif to differentiate nucleic acid (e.g., DNA and/or RNA) associated with viable cells from nucleic acids associated with non-viable cells. The methods do not require a photoactivation step and allow for culture-independent detection of DNA from viable cells with minimal interference from the DNA of dead cells. The multifunctional compounds, compositions, kits, and methods can simplify the vPCR process and improve the consistency and robustness of molecular-detection based viability testing. The multifunctional compounds can also be used in other applications such as methods of removing nucleic acids from samples. In some embodiments, the multifunctional compounds further comprise an affinity and/or conjugation moiety that provides for the isolation or removal of nucleic acids bound by the multifunctional compounds herein from a sample.

In some embodiments, the multifunctional compounds herein comprise RNA binding moieties and are useful in multiple applications including but not limited to: determining viral capsid integrity as a surrogate for viral infectivity, determining amount of mRNA or RNA encapsulation within lipid nanoparticle/exosome or other delivery vectors, and elimination of unwanted RNA from a sample.

Many human or veterinary viral pathogens have an RNA genome. Viral infectivity assays often requiring a BSL3 facility. Such facilities are not very common. Simple highly sensitive and specific methods are required as alternatives. For example, it has been documented that many patients who end up being admitted to a hospital for COVID-19 have high viral load. Although the patients recover and are non-infectious with no culturable virus, nasal samples show up PCR positive even many weeks and months post-recovery. This results in increased burden to the health system. If a PCR-based molecular system existed that is highly specific and sensitive and can also inform if the amplified product is from a virus with compromised membrane (non-infectious), then it would help manage patients in the healthcare system. This could be applicable to many pathogens in addition to SARS-CoV-2.

In vitro transcribed RNA or mRNA encapsulated in lipid nanoparticles (or other delivery system including but not limited to exosomes) is used to make an mRNA vaccine or for use as an RNA transfection in a life science research laboratory setting. It would be highly desirable to know how much of the RNA/mRNA is actually incorporated within a lipid nanoparticle and how much is not. This will help in the design of better lipid nanoparticles, optimization of experimental conditions, etc.

In many circumstances, it may be desirable to eliminate certain classes of RNA. One such example is removal of globin mRNA from blood. Globin mRNA is highly abundant and often interferes with down-stream applications such as next-generation sequencing, array-based hybridization analysis, qPCR, and dPCR, and therefore it is highly desirable to eliminate globin RNA. Enzymatic methods using RNaseH could be harsh on the sample non-enzymatic methods for elimination of the globin mRNA is not ideal. The compounds of the present invention can be used to concomitant crosslink with free globin mRNA from the selective lysis of red blood cells. If the crosslinking molecule also has an affinity tag (e.g., biotin) of conjugation tag (e.g., haloalkane), then the crosslinked free globin RNA can be eliminated using an affinity resin (streptavidin-coated beads) or conjugation agent (e.g., HALOTAG).

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “acyl” refers to a group —C(O)R, wherein R is hydrogen, alkyl, cycloalkyl, heterocyclyl, aryl, heteroalkyl, or heteroaryl. Examples of acyl include, but are not limited to, formyl, acetyl, cyclohexylcarbonyl, and benzoyl.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

As used herein, the term “alkyl” means a straight or branched saturated hydrocarbon chain containing from 1 to 30 carbon atoms, for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 6 to 20 carbon atoms (C₆-C₂₀ alkyl), or 8 to 14 carbon atoms (C₅-C₁₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkylene” refers to a divalent group derived from a straight or branched chain hydrocarbon of 1 to 10 carbon atoms (C₁-C₁₀ alkylene), for example, of 1 to 6 carbon atoms (C₁-C₆ alkylene). Representative examples of alkylene include, but are not limited to, —CH₂—, —CH₂CH₂—, —CH(CH₃)—, —CH₂CH₂CH₂—, —CH₂CH(CH₃)—, —CH₂CH₂CH₂CH₂—, —CH₂CH(CH₃)CH₂—, —CH₂CH₂CH(CH₃)—, —CH₂CH₂CH₂CH₂CH₂—, —CH₂CH(CH₃)CH₂CH₂—, —CH(CH₃)CH₂CH₂CH₂—, —CH₂CH₂CH₂CH₂CH₂CH₂—, —CH₂CH₂CH(CH₃)CH₂CH₂—, —CH₂CH(CH₃)CH₂CH₂CH₂—, and —CH(CH₃)CH₂CH₂CH₂CH₂—.

As used herein, the term “alkenyl” refers to a straight or branched hydrocarbon chain containing from 2 to 30 carbon atoms and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkynyl” refers to a straight or branched hydrocarbon chain containing from 2 to 30 carbon atoms and containing at least one carbon-carbon triple bond. Representative examples of alkynyl include, but are not limited to, ethynyl, propynyl, and butynyl.

As used herein, the term “aryl” refers to an aromatic carbocyclic ring system having a single ring (monocyclic) or multiple rings (bicyclic or tricyclic) including fused ring systems, and zero heteroatoms. As used herein, aryl contains 6-20 carbon atoms (C₆-C₂₀ aryl), 6 to 14 ring carbon atoms (C₆-C₁₄ aryl), 6 to 12 ring carbon atoms (C₆-C₁₂ aryl), or 6 to 10 ring carbon atoms (C₆-C₁₀ aryl). Representative examples of aryl groups include, but are not limited to, phenyl, naphthyl, anthracenyl, and phenanthrenyl.

As used herein, the term “arylene” refers to a divalent aryl group. Representative examples of arylene groups include, but are not limited to, phenylene groups (e.g., 1,2-phenylene, 1,3-phenylene, and 1,4-phenylene).

As used herein, the term “cycloalkyl” refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “cycloalkenyl” means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl, and cycloheptenyl.

As used herein, the term “halogen” or “halo” means F, Cl, Br, or I.

As used herein, the term “haloalkyl” means an alkyl group, as defined herein, in which at least one hydrogen atom (e.g., one, two, three, four, five, six, seven or eight hydrogen atoms) is replaced by a halogen.

As used herein, the term “heteroalkyl” means an alkyl group, as defined herein, in which one or more of the carbon atoms (and any associated hydrogen atoms) are each independently replaced with a heteroatom group such as —NR—, —O—, —S—, —S(O)—, —S(O)₂—, and the like, where R is H, alkyl, aryl, cycloalkyl, heteroalkyl, heteroaryl, or heterocyclyl, each of which may be optionally substituted. By way of example, 1, 2 or 3 carbon atoms may be independently replaced with the same or different heteroatomic group. Examples of heteroalkyl groups include, but are not limited to, —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. Heteroalkyl also includes groups in which a carbon atom of the alkyl is oxidized (i.e., is —C(O)—).

As used herein, the term “heteroalkylene” means an alkylene group, as defined herein, in which one or more of the carbon atoms (and any associated hydrogen atoms) are each independently replaced with a heteroatom group such as —NR—, —O—, —S—, —S(O)—, —S(O)₂—, and the like, where R is H, alkyl, aryl, cycloalkyl, heteroalkyl, heteroaryl or heterocyclyl, each of which may be optionally substituted. By way of example, 1, 2 or 3 carbon atoms may be independently replaced with the same or different heteroatomic group. Heteroalkylene also includes groups in which a carbon atom of the alkyl is oxidized (i.e., is —C(O)—). Examples of heteroalkylene groups include, but are not limited to, —CH₂—O—CH₂—, —CH₂—S—CH₂—, —CH₂—NR—CH₂—, —CH₂—NH—C(O)—CH₂, and the like, as well as polyethylene oxide chains, polypropylene oxide chains, and polyethyleneimine chains.

As used herein, the term “heteroaryl” refers to an aromatic group having a single ring (monocyclic) or multiple rings (bicyclic or tricyclic), having one or more ring heteroatoms independently selected from O, N, and S. The aromatic monocyclic rings are five- or six-membered rings containing at least one heteroatom independently selected from O, N, and S (e.g., 1, 2, 3, or 4 heteroatoms independently selected from O, N, and S). The five-membered aromatic monocyclic rings have two double bonds, and the six-membered aromatic monocyclic rings have three double bonds. The bicyclic heteroaryl groups are exemplified by a monocyclic heteroaryl ring appended fused to a monocyclic aryl group, as defined herein, or a monocyclic heteroaryl group, as defined herein. The tricyclic heteroaryl groups are exemplified by a monocyclic heteroaryl ring fused to two rings independently selected from a monocyclic aryl group, as defined herein, and a monocyclic heteroaryl group as defined herein. Representative examples of monocyclic heteroaryl include, but are not limited to, pyridinyl (including pyridin-2-yl, pyridin-3-yl, pyridin-4-yl), pyrimidinyl, pyrazinyl, pyridazinyl, pyrrolyl, benzopyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, furanyl, oxazolyl, isoxazolyl, 1,2,4-triazinyl, and 1,3,5-triazinyl. Representative examples of bicyclic heteroaryl include, but are not limited to, benzimidazolyl, benzodioxolyl, benzofuranyl, benzooxadiazolyl, benzopyrazolyl, benzothiazolyl, benzothienyl, benzotriazolyl, benzoxadiazolyl, benzoxazolyl, chromenyl, imidazopyridine, imidazothiazolyl, indazolyl, indolyl, isobenzofuranyl, isoindolyl, isoquinolinyl, naphthyridinyl, purinyl, pyridoimidazolyl, quinazolinyl, quinolinyl, quinoxalinyl, thiazolopyridinyl, thiazolopyrimidinyl, thienopyrrolyl, and thienothienyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

As used herein, the term “heterocycle” or “heterocyclic” refers to a saturated or partially unsaturated non-aromatic cyclic group having one or more ring heteroatoms independently selected from O, N, and S, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from O, N, and S. The three- or four-membered ring contains zero or one double bond, and one heteroatom selected from O, N, and S. The five-membered ring contains zero or one double bond and one, two or three heteroatoms selected from O, N, and S. The six-membered ring contains zero, one, or two double bonds and one, two, or three heteroatoms selected from O, N, and S. The seven- and eight-membered rings contains zero, one, two, or three double bonds and one, two, or three heteroatoms selected from O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan,hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom, or any nitrogen atom contained within the rings.

As used herein, the term “hydroxy” means an —OH group.

In some instances, the number of carbon atoms in a group (e.g., alkyl, alkoxy, or cycloalkyl) is indicated by the prefix “Cₓ-C_{y}—,” wherein x is the minimum and y is the maximum number of carbon atoms in the group. Thus, for example, “C₁-C₃-alkyl” refers to an alkyl group containing from 1 to 3 carbon atoms.

As used herein, the term “substituent” refers to a group substituted on an atom of the indicated group.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, thione, or combinations thereof.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— optionally also recites —OCH₂—, and —OC(O)NH— also optionally recites —NHC(O)O—.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, a “nucleic acid binding moiety” or “NAB moiety” is a moiety that interacts with nucleic acid in a non-covalent manner. Non-limiting examples of NAB moieties include major groove and minor groove binders (which interact with DNA by binding to the major or minor groove of the DNA double helix), intercalators (planar moieties that insert between nucleotide base pairs), and mixed-mode NAB moieties (including a portion which intercalates into the DNA double helix and a portion that protrudes into a groove, such as the minor groove). NAB moieties may bind to DNA, RNA, or both.

As used herein, a “DNA binding moiety” or “DAB moiety” is a moiety that specifically interacts with deoxyribonucleic acid (preferentially over RNA) in a non-covalent manner. In some embodiments, a DAB moiety exhibits at least 2-fold (e.g., 2×, 3×, 4×, 5×, 10×, 20×, 100×, 200×, 500×, 100×, or more, or ranges therebetween) increased affinity for DNA over RNA.

As used herein, a “RNA binding moiety” or “RAB moiety” is a moiety that specifically interacts with ribonucleic acid (preferentially over DNA) in a non-covalent manner. In some embodiments, a RAB moiety exhibits at least 2-fold (e.g., 2×, 3×, 4×, 5×, 10×, 20×, 100×, 200×, 500×, 100×, or more, or ranges therebetween) increased affinity for RNA over DNA.

As used herein, a “nucleic acid modifying moiety” or “NAM moiety” is a moiety that reacts with at least one nucleotide of a nucleic acid, forming a covalent linkage to the nucleic acid (e.g., a covalent bond or a coordinate covalent bond). The NAM moiety can form a covalent linkage with one nucleobase, or can react with two different nucleobases to form a crosslink, either within the same strand (intrastrand) or between opposite strands (intrastrand).

As used herein, the term “affinity moiety” refers to a functional group capable of forming a stable non-covalent interaction with an “affinity agent.” Examples of affinity moiety include, but are not limited to, biotin and digoxigenin.

As used herein, the term “conjugation moiety” refers to a functional group capable of forming a covalent bond with a “conjugation agent.” An exemplary conjugation moiety and conjugation agent are chloroalkane and HALOTAG.

As used herein, the term “cell permeable” refers to a compound or moiety that is capable of effectively crossing a cell membrane of a non-viable cell or a cell membrane of a viable cell that has been synthetically permeabilized or the intact membrane of a cell whether viable or non-viable. As used herein, the term “cell impermeable” refers to a compound or moiety that is incapable of effectively crossing a cell membrane of a viable cell that has not been synthetically permeabilized.

As used herein, the term “sample” is used in its broadest sense. In one sense, it is meant to include a specimen or culture obtained from any source as well as biological, food, and environmental samples. Biological samples may be obtained from animals (including humans) and encompass fluids, solids, tissues, and gases. Biological samples include blood products such as plasma, serum, and the like. Sample may also refer to cell lysates, which may include cells that have been lysed with a lysing agent or lysates such as rabbit reticulocyte or wheat germ lysates. Sample may also include cell-free expression systems. Environmental samples include environmental material such as surface matter, soil, water, crystals, and industrial samples. Such examples are not to be construed as limiting the sample types applicable to the present disclosure.

As used herein the term “microorganism” refers to any microscopic organisms and includes viruses, in addition to bacteria, actinomycetales, cyanobacteria (unicellular algae), fungi, protozoa.

As used herein, the term “substantially” means that the recited characteristic, parameter, and/or value need not be achieved exactly, but that deviations or variations, including for example, tolerances, measurement error, measurement accuracy limitations, and other factors known to skill in the art, may occur in amounts that do not preclude the effect the characteristic was intended to provide. A characteristic or feature that is substantially absent may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic.

Unless otherwise defined herein, scientific, and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. COMPOUNDS

The present disclosure includes compounds that can selectively bind to and modify nucleic acids from non-viable cells without requiring a photoactivation step. Such compounds can be used in methods of selectively detecting nucleic acids from viable cells, and in methods of removing nucleic acids from samples.

In one aspect, the disclosure provides a multifunctional compound or a salt thereof, the multifunctional compound comprising:
(A) a nucleic acid binding moiety (“NAB moiety”);
(B) a live/dead cell differentiating moiety (“LDCD moiety”); and
(C) a nucleic acid modifying moiety (“NAM moiety”).

In a second aspect, the disclosure provides a compound or a salt thereof, the multifunctional compound comprising:
(A) a nucleic acid binding moiety (“NAB moiety”);
(B) a live/dead cell differentiating moiety (“LDCD moiety”); and
(C) an affinity moiety.

In a third aspect, the disclosure provides a compound or a salt thereof, the multifunctional compound comprising:
(A) a nucleic acid binding moiety (“NAB moiety”);
(B) a live/dead cell differentiating moiety (“LDCD moiety”); and
(C) a conjugation moiety.

In a fourth aspect, the disclosure provides a compound or a salt thereof, the multifunctional compound comprising:
(A) a nucleic acid binding moiety (“NAB moiety”);
(B) a live/dead cell differentiating moiety (“LDCD moiety”);
(C) a nucleic acid modifying moiety (“NAM moiety”); and
(D) an affinity moiety or a conjugation moiety.

In some embodiments, the multifunctional compound comprises more than one NAB moiety (e.g., two NAB moieties), more than one LDCD moiety (e.g., two LDCD moieties), more than one NAM moiety (e.g., two NAM moieties), more than one conjugation moieties, and/or more than one affinity moieties. In some embodiments, the multifunctional compound comprises more than one NAB moiety (e.g., two NAB moieties). In some embodiments, the multifunctional compound comprises more than one NAM moiety (e.g., two NAM moieties). In some embodiments, the NAB moiety is a DAB moiety. In some embodiments, the NAB moiety is a RAB moiety.

In some embodiments, the multifunctional compound has a structure selected from: A-B—C, B-A-C, A-C—B, B—C-A,

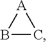

or A-B(D)-C (wherein D is a substituent off of B);

wherein A is the NAB moiety (e.g., RAB moiety or DAB moiety); B is the LDCD moiety; and C is the NAM moiety, affinity moiety, or conjugation moiety. When D is present, C is a NAM moiety, and D is an affinity moiety or conjugation moiety.

In some embodiments, the multifunctional compound has the structure A-B—C, wherein A is the NAB moiety, B is the LDCD moiety, and C is the NAM moiety. In such embodiments, the NAB moiety A is linked to the LDCD moiety B via a covalent bond, and the NAM moiety C is linked to the LDCD moiety B by another covalent bond.

In some embodiments, the multifunctional compound has the structure A-B—C, wherein A is the NAB moiety, B is the LDCD moiety, and C is the affinity moiety. In such embodiments, the NAB moiety A is linked to the LDCD moiety B via a covalent bond, and the affinity moiety C is linked to the LDCD moiety B by another covalent bond.

In some embodiments, the multifunctional compound has the structure A-B—C, wherein A is the NAB moiety, B is the LDCD moiety, and C is the conjugation moiety. In such embodiments, the NAB moiety A is linked to the LDCD moiety B via a covalent bond, and the conjugation moiety C is linked to the LDCD moiety B by another covalent bond.

In some embodiments, the multifunctional compound has the structure A-B(D)-C, wherein A is the NAB moiety, B is the LDCD moiety, C is the NAM moiety, and D is the conjugation moiety. In such embodiments, the NAB moiety A is linked to the LDCD moiety B via a covalent bond, the NAM moiety C is linked to the LDCD moiety B by another covalent bond, and the conjugation moiety D is linked to the LDCD moiety B via a covalent bond.

In some embodiments, the multifunctional compound has the structure A-B(D)-C, wherein A is the NAB moiety, B is the LDCD moiety, C is the NAM moiety, and D is the affinity moiety. In such embodiments, the NAB moiety A is linked to the LDCD moiety B via a covalent bond, the NAM moiety C is linked to the LDCD moiety B by another covalent bond, and the affinity moiety D is linked to the LDCD moiety B via a covalent bond.

The multifunctional compounds include at least one NAB moiety (e.g., a RAB or DAB moiety), which is a moiety that interacts with nucleic acids in a non-covalent manner. The NAB moiety efficiently binds to nucleic acid species, facilitating rapid covalent modification of the nucleic acid by the NAM moiety. In some embodiments, the NAB moiety is a DAB moiety that interacts with DNA by binding to the major groove of the DNA double helix. In some embodiments, the NAB moiety is a DAB moiety that interacts with DNA by binding to the minor groove of the DNA double helix. In some embodiments, the NAB moiety is an intercalating moiety that inserts between nucleotide base pairs, such as an intercalating dye. In some embodiments, the NAB moiety is a RAB moiety that interacts with RNA.

Suitable NAB moieties comprise groups such as acridine, phenanthridine (e.g., phenylphenanthridium), dipyridine, terpyridine, phenanthroline, indole, quinoline, cyanine, quinacrine, benzothiazole, benzimidazole (e.g., bibenzimidazole), pyridocarbazole (e.g., a pyridocarbazole dimer), an aminoglycoside, and the like. Furthermore, a wide variety of nucleic acid stains are known, any of which (or portions thereof) can be used as the NAB moiety in the multifunctional compounds described herein. Examples of nucleic acid stains include, for example, ethidium bromide, Hoescht stain, DAPI, and SYBR Green. Additional nucleic acid stains are described in the Molecular Probes Handbook, a Guide to Fluorescent Probes and Labeling Technologies, 11^{th} Edition (2010), Chapter 8, which is incorporated herein by reference in its entirety. Other nucleic acid binding dyes are disclosed in U.S. Pat. No. 9,206,474, which is incorporated herein by reference in its entirety. Other compounds known to have nucleic acid binding activity include metallo-intercalators, such as ruthenium and rhodium complexes with ligands such as bipyridine, phenanthroline, 4,4,-diphenylbipyridine, and derivatives thereof.

Representative examples of NAB moieties that can be used in the multifunctional compounds described herein include the following.

RNA binding moieties, such as a moiety of formula (I):

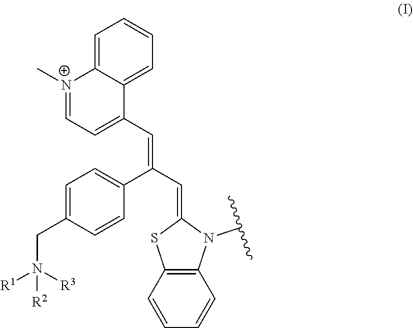

or a tautomer or a salt thereof, wherein:

R¹ and R² are each independently selected from hydrogen, C₁-C₆ alkyl, C₂-C₆ alkenyl, C₂-C₆ alkynyl, aryl, and aryl-C₁-C₆ alkyl, each of which is optionally substituted with 1-3 substituents, or R¹ and R² are taken together with the nitrogen atom to which they are attached to form an optionally substituted heterocyclyl or heteroaryl ring; and

R³ is absent or is C₁-C₆ alkyl.

In some embodiments, R¹ and R² are each independently selected from C₁-C₆ alkyl, and R³ is absent or is C₁-C₆ alkyl. In some embodiments, R¹, R², and R³ are each independently selected from C₁-C₆ alkyl (e.g., methyl or ethyl). In some embodiments, R¹ and R² are each independently selected from C₁-C₆ alkyl (e.g., methyl or ethyl), and R³ is absent. In some embodiments, R³ is absent, and R¹ and R² are taken together with the nitrogen atom to which they are attached to form an optionally substituted monocyclic 5- or 6-membered heterocyclyl or heteroaryl ring having 1, 2, or 3 heteroatoms independently selected from N, O, and S. In some embodiments, R³ is absent, and R¹ and R² are taken together with the nitrogen atom to which they are attached to form a pyrrolidinyl, piperidinyl, morpholino, piperazinyl, or imidazolyl ring, each of which is optionally substituted with one substituent (e.g., C₁-C₆ alkyl, such as methyl). In some embodiments, the group —NR¹R²R³ in the moiety of formula (I) has a formula selected from:

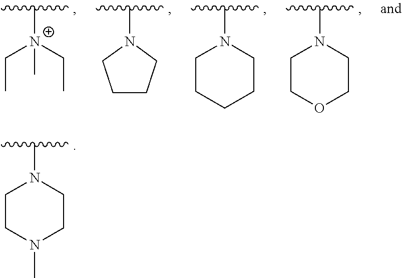

Exemplary RAB moieties include:

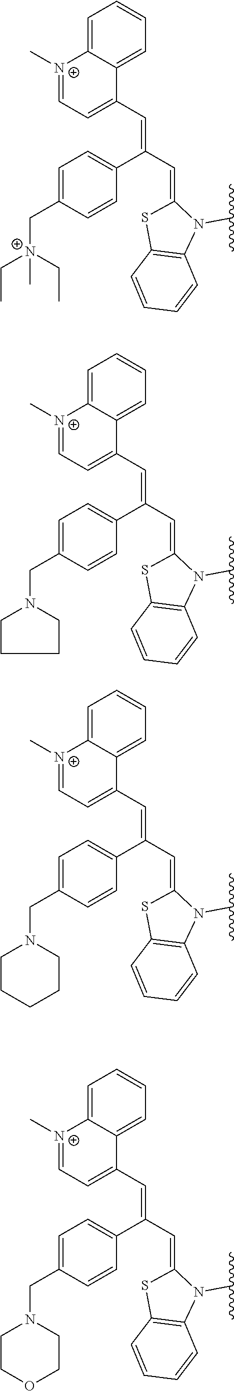 [SMILES: *N1/C(=C/C(=C/c2cc[n+](C)c3ccccc23)c2ccc(C[N+](C)(CC)CC)cc2)Sc2ccccc21 ; *N1/C(=C/C(=C/c2cc[n+](C)c3ccccc23)c2ccc(CN3CCCC3)cc2)Sc2ccccc21 ; *N1/C(=C/C(=C/c2cc[n+](C)c3ccccc23)c2ccc(CN3CCCCC3)cc2)Sc2ccccc21 ; *N1/C(=C/C(=C/c2cc[n+](C)c3ccccc23)c2ccc(CN3CCOCC3)cc2)Sc2ccccc21]

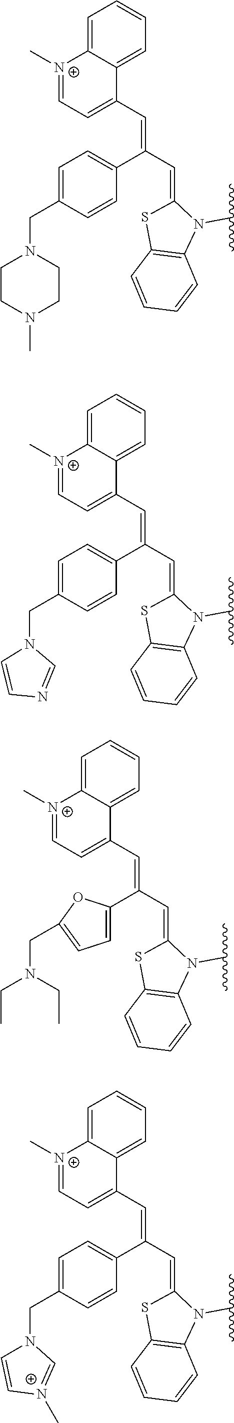 [SMILES: *N1/C(=C/C(=C/c2cc[n+](C)c3ccccc23)c2ccc(CN3CCN(C)CC3)cc2)Sc2ccccc21 ; *N1/C(=C/C(=C/c2cc[n+](C)c3ccccc23)c2ccc(Cn3ccnc3)cc2)Sc2ccccc21 ; *N1/C(=C/C(=C/c2cc[n+](C)c3ccccc23)c2ccc(CN(CC)CC)o2)Sc2ccccc21 ; *N1/C(=C/C(=C/c2cc[n+](C)c3ccccc23)c2ccc(Cn3cc[n+](C)c3)cc2)Sc2ccccc21]

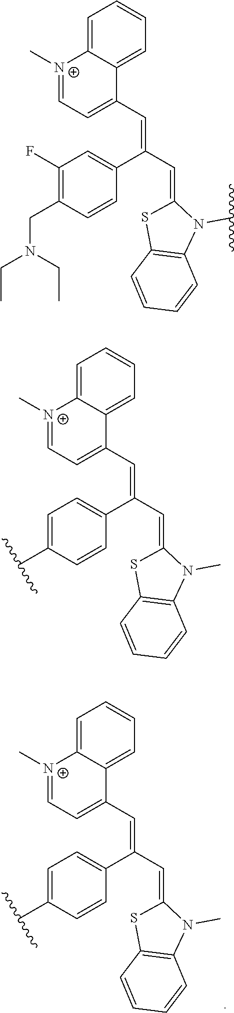

Single-stranded DNA/RNA binding moieties, such as:

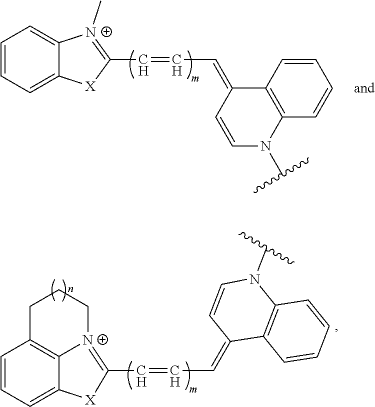

wherein X is O, NH, or S, m is 0, 1, or 2, and n is 0, 1, 2, or 3 (e.g., wherein m is 1 and n is 1);

Intercalating moieties, such as:

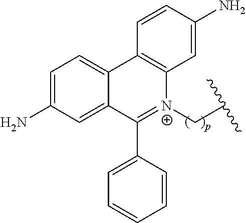

wherein p is 0 or 1,

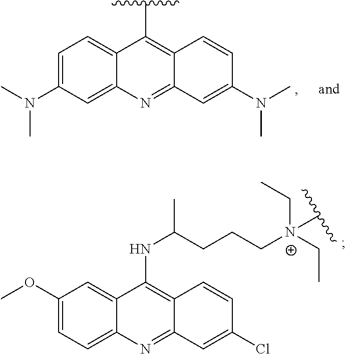

Minor groove binders, such as:

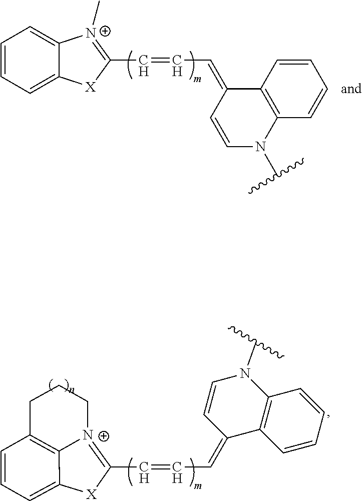

wherein X is O, NH, or S, m is 0, 1, or 2, and n is 0, 1, 2, or 3 (e.g., wherein m is 1 and n is 1);

Major groove binders, such as:

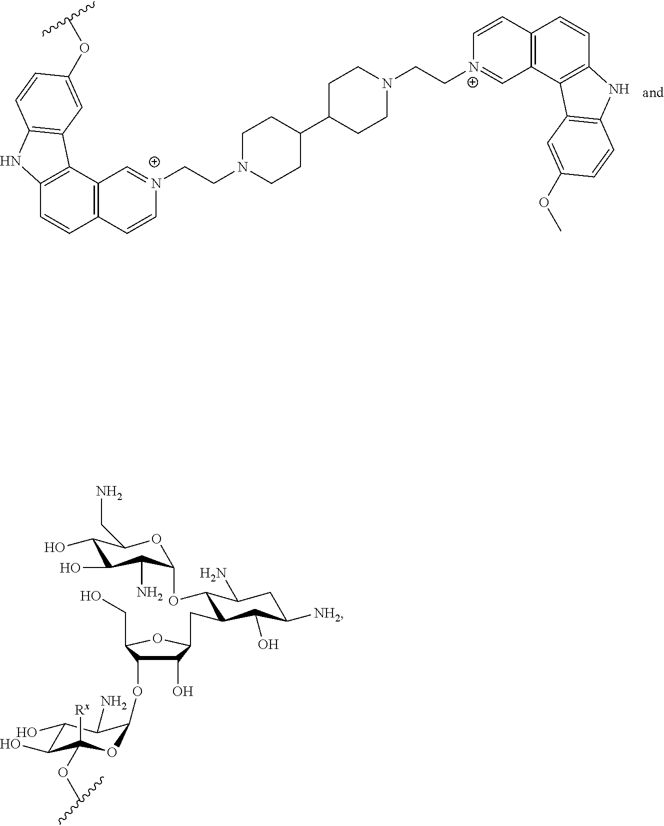

wherein R^{x} is —CH₂NH₂ or H.

In particular embodiments, the NAB moiety is selected from:

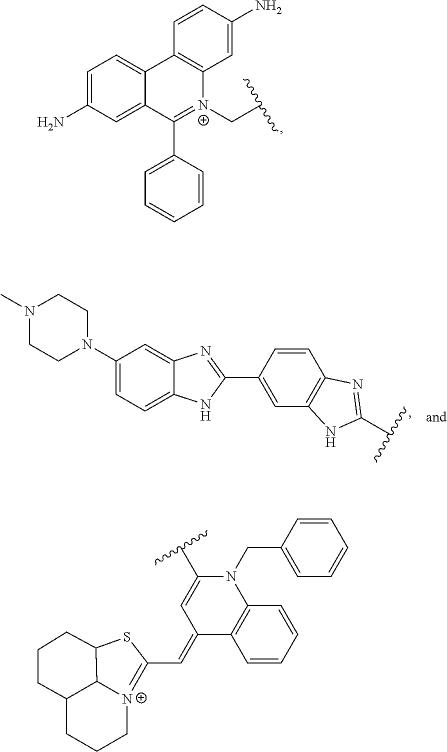 [SMILES: *C[n+]1c(-c2ccccc2)c2cc(N)ccc2c2ccc(N)cc21 ; *c1nc2ccc(-c3nc4cc(N5CCN(C)CC5)ccc4[nH]3)cc2[nH]1 ; *C1=C/C(=C\C2=[N+]3CCCC4CCCC(S2)C43)c2ccccc2N1Cc1ccccc1]

The multifunctional compounds include an LDCD moiety, which comprises at least one LDCD motif that renders the multifunctional compounds capable of binding to and modifying the DNA from dead cells, but not viable cells. The LDCD motif either prevents entry of the molecule into live cells or is processed by live cells such that the multifunctional compound is unable to modify viable cell DNA. The LDCD motif distinguishes the multifunctional compounds described herein from other compounds that include a NAB moiety and a NAM moiety (e.g., anticancer drugs or research agents, in which the multifunctional compounds are designed to be cell permeable in order to bind to and modify nucleic acids in live cells). In addition to the at least one LDCD motif, the LDCD moiety can also include other atoms or groups of atoms, including but not limited to alkylene groups (—(CH₂)ₙ—), ether groups (—O—), thioether groups (—S—), amide linkages (—C(O)NH—), ester linkages (—C(O)O—), carbamate linkages (—OC(O)NH—), sulfonamide linkages (—S(O)₂NH—), phenylene linkages (—C₆H₄—), and any combination thereof. Furthermore, any substitutable atom or group can be substituted with an appropriate substituent (e.g., an amide linkage could include a group —C(O)NR—, where R is a suitable substituent, or a phenylene linkage can include one or more substituents on the phenyl group).

In some embodiments, the LDCD moiety serves as a linker between one or more NAB moieties and one or more NAM moieties.

In some embodiments, the LDCD moiety comprises at least one LDCD motif selected from charged moieties, high molecular weight moieties, immobilization moieties, and metabolically cleavable moieties.

In some embodiments, the LDCD motif is a charged moiety, which will preclude the molecule from entering live cells. In some embodiments, the LDCD moiety includes at least one charged moiety selected from a carboxylate group, a sulfate group, a sulfonate group, a phosphate group, a phosphonate group, and an ammonium group (e.g., a quaternary ammonium group). In some embodiments, the LDCD moiety includes more than one charged moiety. For example, in some embodiments, the LDCD moiety includes more than one carboxylate group, more than one sulfate group, more than one sulfonate group, more than one phosphate group, more than one phosphonate group, or more than one ammonium group (e.g., more than one quaternary ammonium group). In some embodiments, the LDCD moiety includes 2, 3, 4, 5, 6, or more charged moieties. In some embodiments, the LDCD moiety includes at least one quaternary ammonium center. In some embodiments, the LDCD moiety includes at least one group of formula —N⁺(CH₃)₂. In some embodiments, the LDCD moiety includes at least one carboxylate group. In some embodiments, the LDCD moiety includes at least one group of formula —CH₂COO⁻ or —CH₂CH₂COO—. In some embodiments, the LDCD moiety includes at least one phosphonate group. In some embodiments, the LDCD moiety includes at least one group of formula —CH₂PO₃²— or CH₂CH₂PO₃²⁻.

In some embodiments, the LDCD motif is a high molecular weight moiety, for example, a moiety that has a molecular weight such that the overall compound has a molecular weight of greater than 600 g/mol, e.g., greater than 700 g/mol, greater than 800 g/mol, greater than 900 g/mol, or greater than 1000 g/mol. Compounds of higher molecular weights can slow down mobility and impact cell permeability. The high molecular weight moiety can include any combination of groups such as alkylene, heteroalkylene, arylene, and heteroarylene moieties, provided that the total molecular weight of the LDCD moiety is sufficient to render the multifunctional compound unable to enter a live cell (e.g., such that the overall compound has a molecular weight of 600 g/mol or higher). In some embodiments, the high molecular weight moiety includes a polyethylene glycol chain (i.e., a group of formula —(CH₂CH₂O)ₙ—, wherein n is an integer from 1-100, e.g., 1, 2, 3, 4, 5, 6, 7, 8, 9, 10, 15, 20, 25, 30, 35, 40, 45, 50, 55, 60, 65, 70, 75, 80, 85, 90, 95, or 100). In some embodiments, the LDCD moiety is a peptide, such as a peptide with properties that render the molecule cell-impermeable and do not interfere with DNA binding.

In some embodiments, the LDCD motif is a metabolically cleavable moiety. Molecules having such moieties may be able to enter both viable and non-viable cells, but the viable cells would process the moiety to release the NAM moiety and thus prevent covalent modification. Non-viable cells are not able to process the moiety, therefore the NAM moiety can effectively interact with the nucleic acid to prevent its amplification. For example, a trimethyl quinone lock is a metabolically cleavable moiety that, when incorporated into compounds at an appropriate position, can be processed inside a viable cell to release the NAB moiety or the NAM moiety, rendering the multifunctional compound unable to target and modify the DNA. For example, in some embodiments, in a viable cell, the trimethylquinone moiety is reduced by an intracellular reductase enzyme. The resulting trimethylhydroquinone moiety undergoes a lactonization reaction to produce a dihydrocoumarin. Further rearrangement of the rest of the molecule then leads to release of the NAM moiety and/or the NAB moiety. See, for example, WO 2015/116867, and Mustafa et al. Bioconjugate Chem. 2016, 27, 1, 87-101, each of which is incorporated herein by reference in its entirety. An exemplary reaction is shown below in Scheme 1.

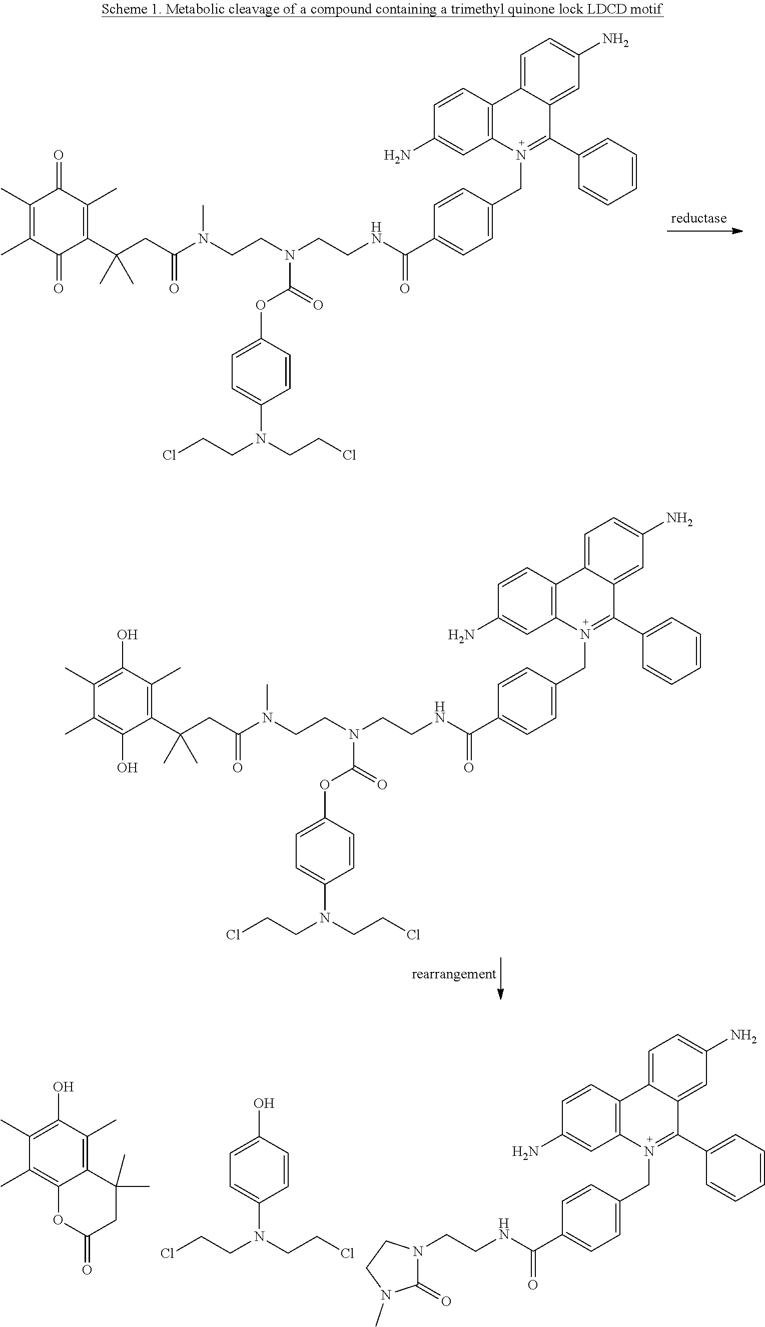

In some embodiments, the LDCD motif comprises a solid support. Binding of the multifunctional compound to a solid support allows a sample containing viable and non-viable cells to be mixed with the solid support such that DNA from non-viable cells will bind to the solid support, whereas viable cells will not. Separation of the solid support from the rest of the sample will provide a sample containing only viable cells, which can be used in a subsequent amplification reaction. This embodiment also allows for removal of free DNA from other types of samples, which can be used in applications requiring generation of DNA-free reagents and/or solutions. The solid support could be, for example, a bead, a resin, a magnetic particle, a membrane, a gel, an ionic liquid (see, e.g., Egorova et al. Chem. Rev. 2017, 117, 10, 7132-7189), or a surface such as the surface of a tube, vial, slide, microtiter plate, cuvette, or the like. Methods of immobilizing compounds on solid supports are known to those skilled in the art.

In some embodiments, the LDCD motif comprises one or more of the following groups:

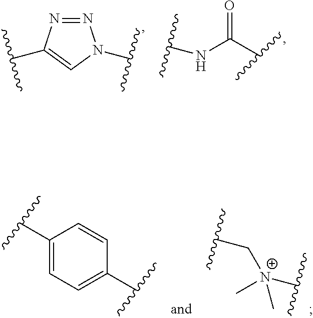

and; and may also include, but are not limited to alkylene groups (—(CH₂)ₙ—), ether groups (—O—), thioether groups (—S—), ester linkages (—C(O)O—), carbamate linkages (—OC(O)NH—), sulfonamide linkages (—S(O)₂NH—), and any combination thereof. Exemplary LDCD moieties include, but are not limited to:

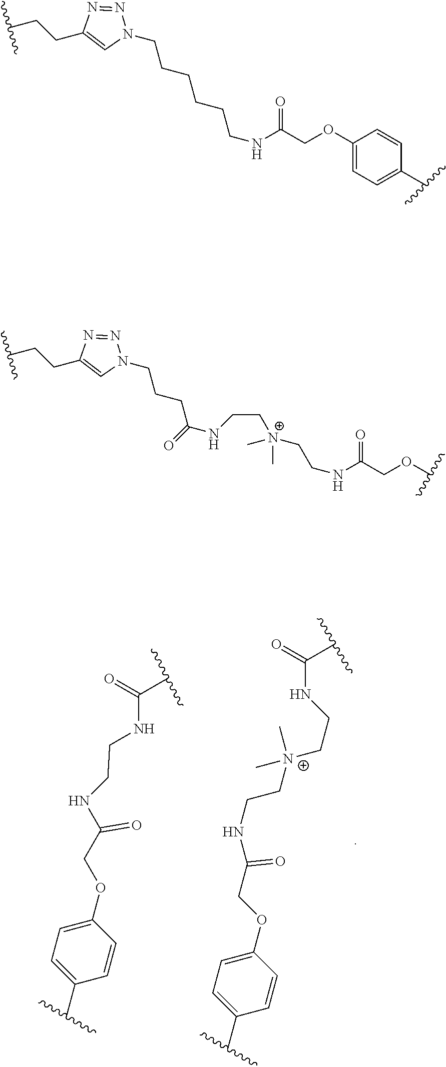 [SMILES: *CCc1cn(CCCCCCNC(=O)COc2ccc(*)cc2)nn1 ; *CCc1cn(CCCC(=O)NCC[N+](C)(C)CCNC(=O)CO*)nn1 ; *C(=O)NCCNC(=O)COc1ccc(*)cc1 ; *C(=O)NCC[N+](C)(C)CCNC(=O)COc1ccc(*)cc1]

In some embodiments, an LDCD is branched, allowing for the connecting of three or more other moieties via the LDCD (e.g., a NAB moiety, a NAM moiety, and an affinity or conjugation moiety). For example, a LDCD may contain one of the following functional groups:

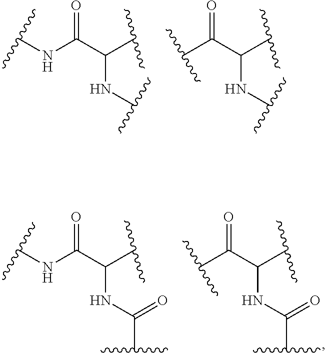 [SMILES: *NC(=O)C(*)N* ; *NC(*)C(*)=O ; *NC(=O)C(*)NC(*)=O ; *C(=O)NC(*)C(*)=O]

in addition to the aforementioned groups that comprise an LDCD. An exemplary branched LDCD that finds use in embodiments herein is:

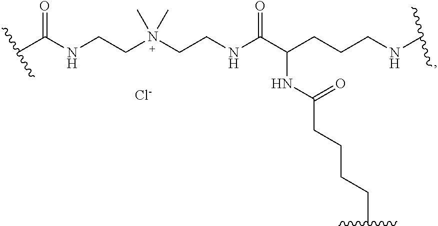

wherein three moieties described herein are attached thereto (e.g., one more NAB moieties, one more NAM moieties, one more affinity moieties, one more conjugation moieties (e.g., a NAM moiety, a NAB moiety, and an affinity or conjugation moiety). An exemplary compound within the scope herein and comprising a NAM moiety, a NAB moiety, an affinity moiety, and a branched LDCD is:

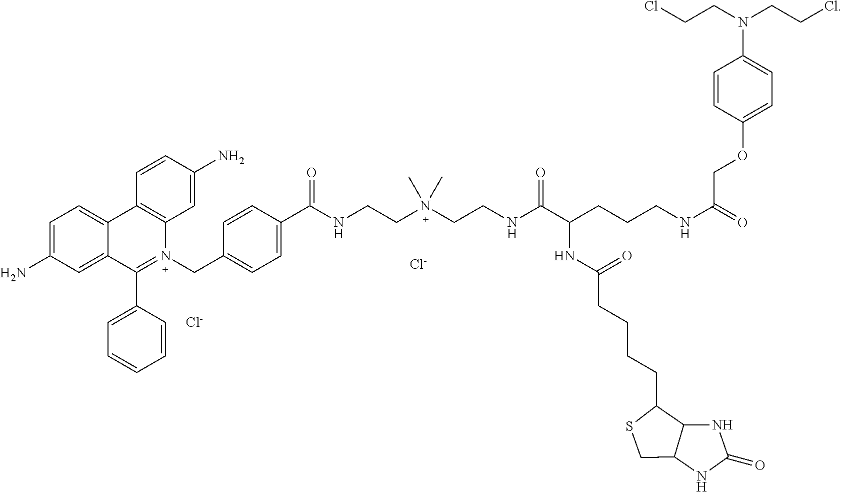

A compound herein may comprise 2 or more branchpoints, allowing for connection of more than three moieties (e.g., 4, 5, 6, 7, 8, 9, 10, or more).

The multifunctional compounds also include at least one NAM moiety, which serves to covalently modify the nucleic acid and thus prevent it from being amplified. Examples of NAM moieties include DNA alkylating agents, such as nitrogen mustards (e.g., bendamustine, chlorambucil, chlormethine, cyclophosphamide, ifosfamide, melphalan, and uramustine), nitrosoureas (e.g., carmustine, chlorozotocin, ethylnitrosourea, fotemustine, lomustine, nimustine, ranimustine, semustine, and streptozocin), and alkyl sulfonates (e.g., busulfan), seco-CBI compounds (e.g., 1-(chloromethyl)-5-hydroxy-1,2-dihydro-3H-benz(e)indole), and pyrrolo[2,1-c][1,4]benzodiazepine (PBD) compounds (e.g., 8-hydroxy-7-methoxy-2-methylene-1,2,3,11a-tetrahydro-5H-benzo[e]pyrrolo[1,2-a][1,4]diazepin-5-one). Any of these, or derivatives thereof, can be used as a basis for a NAM moiety in the multifunctional compounds described herein.

Other NAM moieties include platinum-based moieties, which modify DNA through binding of DNA nucleobases (typically via the N7 position of guanine residues) to the platinum center (via a coordinate covalent bond). Examples of platinum-based chemotherapeutic agents include cisplatin, carboplatin, nedaplatin, ormaplatin, oxaliplatin, phenanthriplatin, picoplatin, and satraplatin, any of which (or derivatives thereof) can be used as a basis for a NAM moiety in the multifunctional compounds described herein. For example, the multifunctional compound may feature a monodentate or bidentate ligand that binds to the platinum center (e.g., a moiety with one or more primary amines and/or secondary amines, and/or another source of a coordinating nitrogen atom, such as a pyridine, quinoline, or phenanthridine moiety).

In some embodiments, the NAM moiety comprises a group selected from:

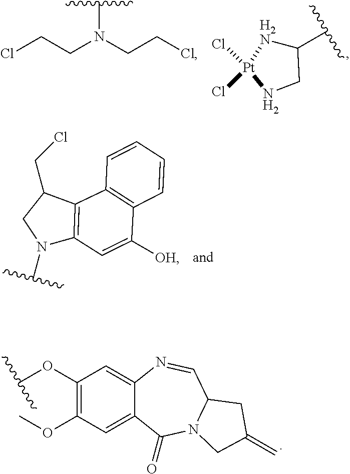

In some embodiments, the multifunctional compound is selected from:

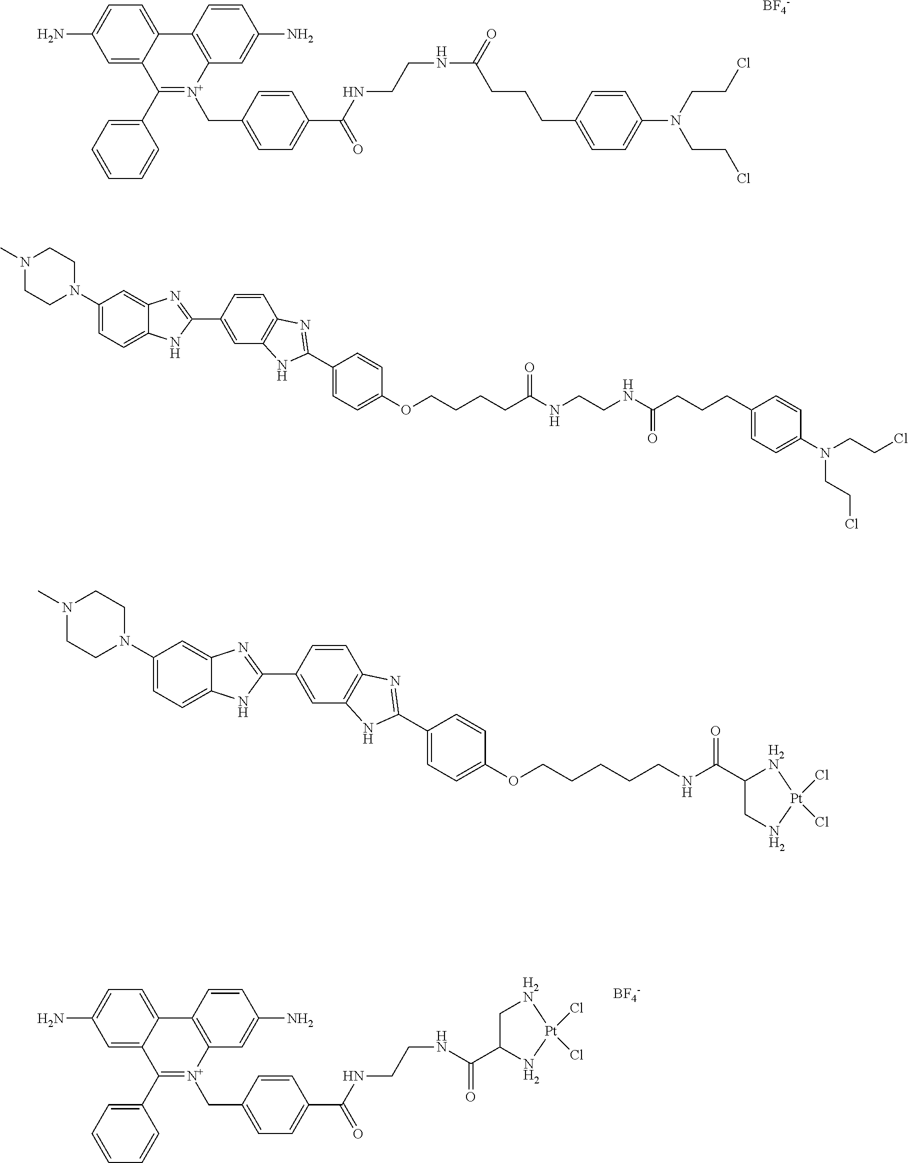

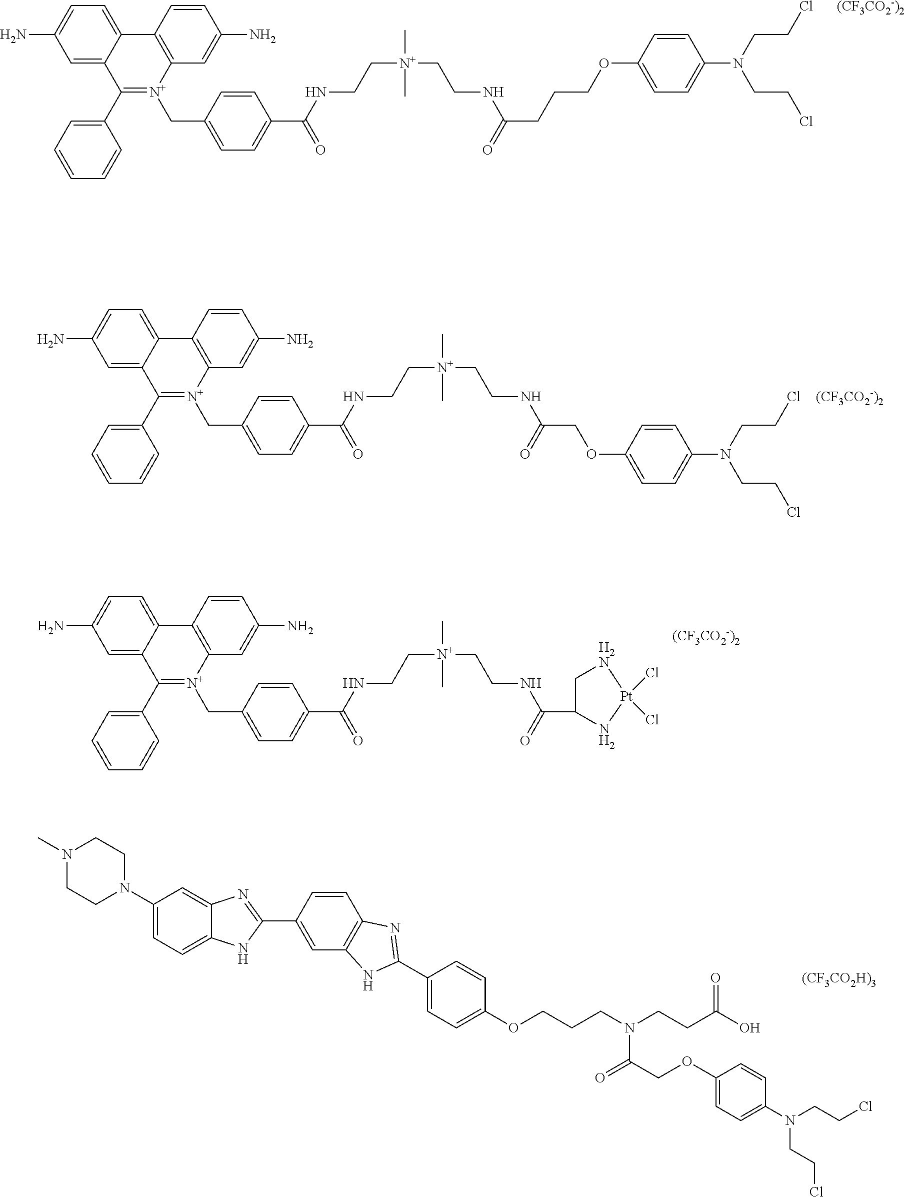

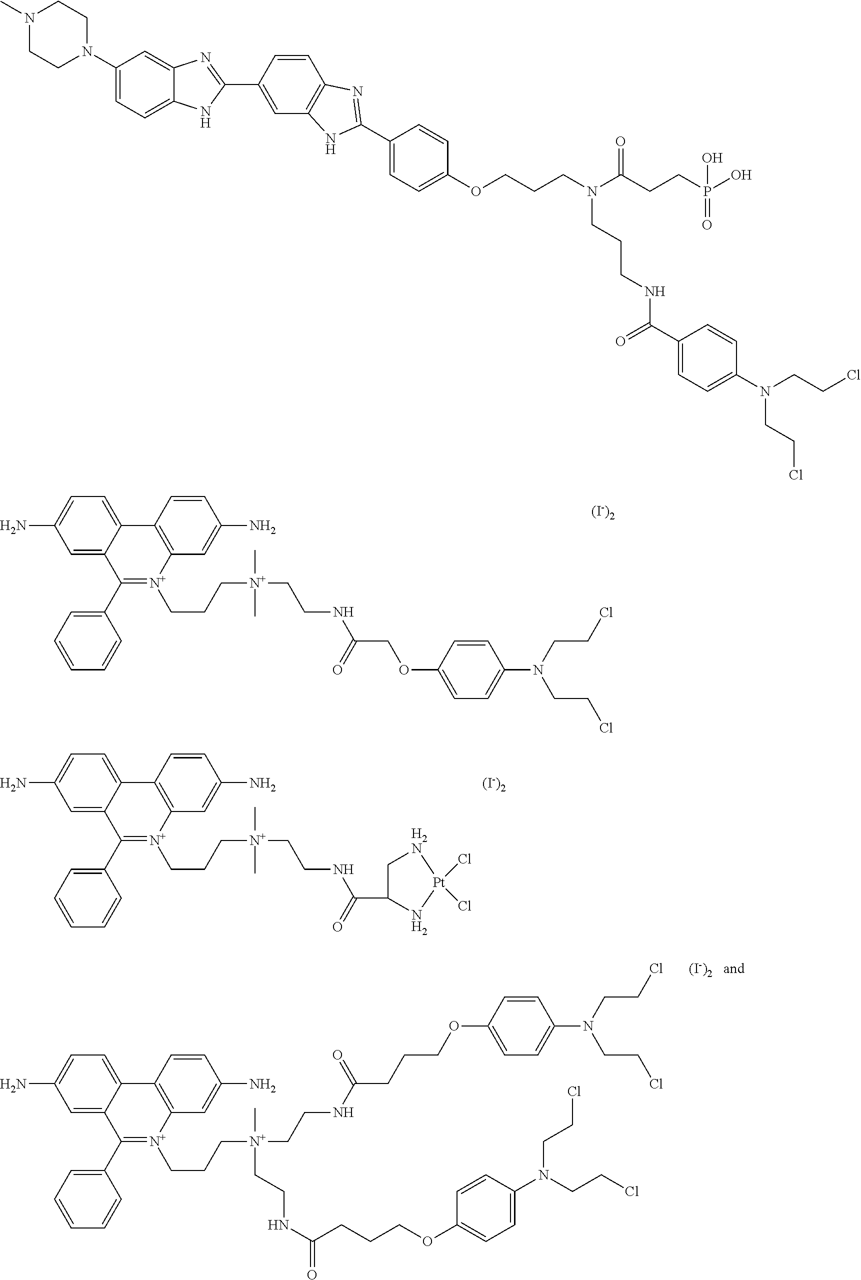

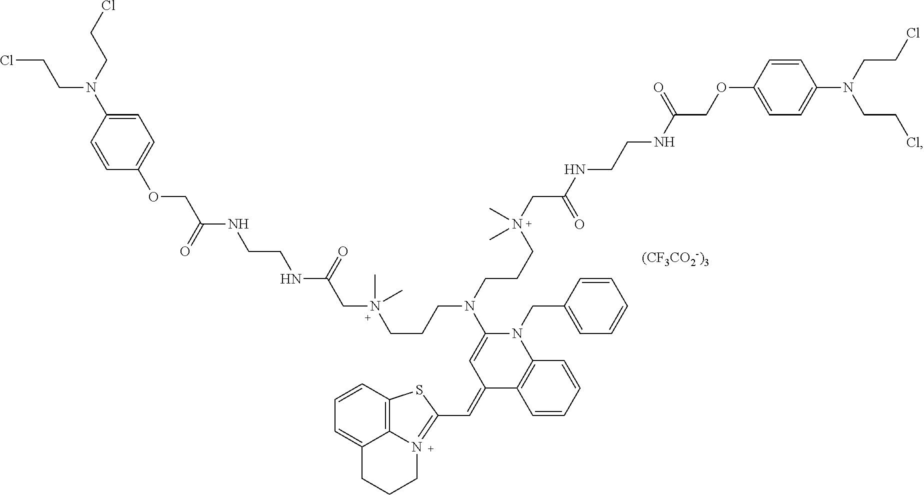

or a salt of any thereof.

In some embodiments, the multifunctional compounds herein comprise an affinity moiety. Such moieties allow the multifunctional compounds to be non-covalently bound by a corresponding affinity agent. Exemplary affinity moieties that find use in compounds herein include biotin, a peptide tag (e.g., 5×His (HHHHH)(SEQ ID NO: 1), 6×His (HHHHHH)(SEQ ID NO: 2), C-myc (EQKLISEEDL) (SEQ ID NO: 3), Flag (DYKDDDDK) (SEQ ID NO: 4), SteptTag (WSHPQFEK)(SEQ ID NO: 5), HA Tag (YPYDVPDYA) (SEQ ID NO: 6), or an epitope. In some embodiments, systems are provided comprising (1) an affinity agent and (2) a compound herein comprising a corresponding affinity moiety. Suitable pairs of affinity moieties and affinity agents for use in embodiments herein include biotin and streptavidin, a His tag and metal ions (e.g., Ni²⁺), FALG tag and antibody, etc.

In some embodiments, the multifunctional compounds herein comprise a conjugation moiety. Such moieties allow the multifunctional compounds to be covalently bound by a corresponding conjugation agent. An exemplary conjugation moiety that finds use in compounds herein include a haloalkane (e.g., choroalkane). In some embodiments, haloalkane moieties are capable of being covalently bound by HALOTAG (Promega Corp.) or other dehalogenase proteins that have been modified to form a stable (e.g., covalent) bond (e.g., ester bond) with a haloalkyl substrate, rather than releasing the halogenated substrate. In some embodiments, haloalkanes that find use as conjugation moieties herein are of the structure —(CH₂)ₙ—Y, wherein n is 2, 3, 4, 5, 6, 7, 8, 9, 10, 11, or 12, and Y is a halogen (i.e., F, Cl, Br, or I). In some embodiments, n is 4, 5, 6, 7, or 8, and Y is Cl. In some embodiments, n is 6 and Y is Cl, such that Q has formula —(CH₂)₆—Cl. The conjugation moiety may also comprise a linker portion that includes various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo- and poly-ethylene glycol (—(CH₂CH₂O)ₓ—) linkages, and the like. In some embodiments, the linker has a formula —O(CH₂CH₂O)_{z1}—C(O)NH—(CH₂CH₂O)_{z2}—C(O)NH—(CH₂)_{z3}—(OCH₂CH₂)_{z4}O—, wherein z1, z2, z3, and z4 are each independently selected form 0, 1, 2, 3, 4, 5, and 6. In some embodiments, systems are provided comprising (1) a modified dehalogenase conjugation agent and (2) a compound herein comprising a haloalkane conjugation moiety.

In some embodiments, a suitable conjugation moiety and conjugation agent pair utilize Staudinger ligation, amide coupling, methods that employ activated esters, imine bond formation (with and without ortho-boronic acid), boronic acid/diol interactions, disulfide bond formation, copper/copper-free azide, diazo and tetrazine “click” chemistry, UV promoted thiolene conjugation, diazirine photolabeling, Diels-Alder cycloaddition, metathesis reaction, Suzuki cross-coupling, thiazolidine (Step-4) coupling, etc. In some embodiments, systems are provided comprising (1) a conjugation agent and (2) a compound herein comprising a corresponding conjugation moiety.

The multifunctional compound can be in the form of a salt. In some embodiments, a neutral form of the multifunctional compound may be regenerated by contacting the salt with a base or acid and isolating the parent compound in a conventional manner. The parent form of the multifunctional compound differs from the various salt forms in certain physical properties, such as solubility in polar solvents, but otherwise the salts are equivalent to the parent form of the multifunctional compound for the purposes of this disclosure.

In particular, if the multifunctional compound is anionic or has a functional group that may be anionic (e.g., —COOH may be —COO—, —SO₃H may be —SO₃, or —P(O)(OH)₂ can be —PO₃²), then a salt may be formed with one or more suitable cations. Examples of suitable inorganic cations include, but are not limited to, alkali metal cations such as Li⁺, Na⁺, and K⁺, alkaline earth cations such as Ca²⁺ and Mg²⁺, and other cations. Sodium salts may be particularly suitable. Examples of suitable organic cations include, but are not limited to, ammonium ion (i.e., NH₄⁺) and substituted ammonium ions (e.g., NH₃R₁⁺, NH₂R₂⁺, NHR₃⁺, and NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine as well as amino acids such as lysine and arginine. In some embodiments, the multifunctional compound is a sodium salt.

If the multifunctional compound is cationic or has a functional group that may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, tetrafluoroboric, toluenesulfonic, trifluoromethanesulfonic, and valeric. In some embodiments, the multifunctional compound is a halide salt, such as a chloro, bromo, or iodo salt. In some embodiments, the multifunctional compound is a tetrafluoroborate or trifluoromethanesulfonate salt.

The multifunctional compounds can be prepared by a variety of methods, including those shown in the Examples. The multifunctional compounds and intermediates herein may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups, and the methods for protecting and deprotecting different substituents using such suitable protecting groups, are well known to those skilled in the art; examples of which can be found in the treatise by PGM Wuts entitled “Greene's Protective Groups in Organic Synthesis” (5th ed.), John Wiley & Sons, Inc. (2014), which is incorporated herein by reference in its entirety. Synthesis of the multifunctional compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step) or by resolution of a mixture of the stereoisomers of the multifunctional compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material or by resolution of a mixture of the geometric isomers of the multifunctional compound or intermediates using a standard procedure such as chromatographic separation.

The synthetic schemes and specific examples as described are illustrative and are not to be read as limiting the scope of the invention as it is defined in the claims. All alternatives, modifications, and equivalents of the synthetic methods and specific examples are included within the scope of the claims.

### 3. COMPOSITIONS AND SYSTEMS/KITS

In some embodiments, the present disclosure provides compositions and systems/kits comprising a multifunctional crosslinking compound described herein (i.e., a compound comprising at least a NAB moiety, a LDCD moiety, and a NAM moiety).

The composition comprising the multifunctional crosslinking compound described herein can further comprise a solvent. In some embodiments, the solvent is water. In such embodiments, the composition may further include one or more water-soluble components such as a salt or a buffer. In some embodiments, the solvent is an organic solvent such as dimethylsulfoxide (DMSO). Use of DMSO as a solvent for the multifunctional compounds may provide additional advantages when amplifying DNA from samples comprising Gram-negative bacteria, because such bacteria have an additional lipopolysaccharide layer that may be more difficult for the multifunctional compounds to cross, even when the cells are non-viable. Use of DMSO as a solvent can increase the DNA modification efficiency in samples containing Gram-negative bacteria.

The present disclosure further provides a system or kit comprising a compound described herein (i.e., a compound comprising a NAB moiety, a LDCD moiety, and a NAM moiety). The system or kit includes the multifunctional compound, either alone or in a solvent such as water or DMSO. When the multifunctional compound is provided alone, the system or kit may further include the solvent in which the multifunctional compound can be dissolved. The system or kit may further comprise one or more reagents used to carry out an amplification reaction, such as a viability PCR reaction or viability RT-PCR reaction.

In some embodiments, systems or kits further comprise a DNA polymerase. DNA polymerases that can be used in accordance with these embodiments include, but are not limited to, any polymerase capable of replicating a DNA molecule. In some embodiments, the DNA polymerase is a thermostable polymerase, which is especially useful in PCR applications. Thermostable polymerases are isolated from a wide variety of thermophilic bacteria, such as Thermus aquaticus (Taq), Thermus brockianus (Tbr), Thermus filiformis (Tfi), Thermus flavus (Tfl), Thermococcus kodakaraenis (KOD), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus horikossii (Pho), Pyrococcus woesei (Pwo), Pyrococcus strain ES4 (ES4), and other species of the Pyrococcus genus, Bacillus caldophilus (Bca), Bacillus sterothermophilus (Bst), Sulfolobus acidocaldarius (Sac), Sulfolobus solfataricus (Sso), Pyrodictium occultum (Poc), Pyrodictium abyssi (Pab), and Methanobacterium thermoautotrophicum (Mth), and mutants, variants, or derivatives thereof. Accordingly, in some embodiments, the system or kit comprises a thermostable DNA polymerase selected from Taq, Tbr, Tfi, Tfl, KOD, Tru, Tth, Tli, Tac, Tne, Tma, Pfu, Pho, Pwo, ES4, Bca, Bst, Sac, Sso, Poc, Pab, and Mth, or a mutant, variant, or derivative of any thereof. In some embodiments, the DNA polymerase is Taq polymerase. In other embodiments, the DNA polymerase is a polymerase having strand displacement activity, which are especially useful in isothermal amplification reactions. DNA polymerases having strand displacement activity are isolated from a variety of organisms, such as Bacillus smithii (Bsm), Bacillus stearothermophilus (Bst), Bacillus subtilis (Bsu), and Bacillus subtilis phage phi29 (phi29), and mutants, variants, or derivatives thereof. Accordingly, in some embodiments, the system or kit comprises a DNA polymerase selected from Bsm, Bst, Bsu, and phi29, or a mutant, variant, or derivative of any thereof.

In some embodiments, DNA polymerases that can be used in accordance with these embodiments include, but are not limited to, commercially available DNA polymerases (e.g., from Boehringer Mannheim Corp., Indianapolis, IN; Life Technologies, Inc., Rockville, MD; MilliporeSigma, St. Louis, MO; New England Biolabs, Inc., Beverley, MA; Perkin Elmer Corp., Norwalk, CT; Pharmacia LKB Biotechnology, Inc., Piscataway, NJ; Promega Corporation, Madison, WI; Qiagen, Inc., Valencia, CA; and Stratagene, La Jolla, CA).

In some embodiments, systems or kits further comprise a reverse transcriptase. In some embodiments, the reverse transcriptase may have intrinsic RNase H activity, which typically is favored in quantitative PCR applications because they enhance the melting of RNA-DNA duplex during the first cycles of PCR. A variety of reverse transcriptases suitable for RT-qPCR are known in the art and may be used in the disclosed systems and methods. In some embodiments, M-MLV reverse transcriptase from the Moloney murine leukemia virus or AMV reverse transcriptase from the avian myeloblastosis virus are used in quantitative RT-PCR applications. M-MLV reverse transcriptase is a preferred reverse transcriptase in cDNA synthesis for long messenger RNA (mRNA) templates (>5 kb) because the RNase H activity of M-MLV reverse transcriptase is weaker than the AMV reverse transcriptase (see, e.g., Mo et al., Methods Mol Biol., 926: 99-112 (2012)). Thermostable RNAse H-RTs also have been recently developed and may be used in connection with the systems and methods described herein.

In some embodiments, the system or kit further comprises one or more additional reagents, such as at least one primer or at least one pair of primers for amplification of a nucleic acid target, at least one probe and/or dye to enable detection of amplification, a buffer, a ligase, a detergent (e.g., non-ionic detergents), nucleotides (dNTPs and/or NTPs), a magnesium salt (e.g., magnesium chloride), or any combination thereof, among other amplification reagents that would be recognized by one of ordinary skill in the art based on the present disclosure.

In some embodiments, the system or kit comprises one or more primers. Generally, a primer is a shorter nucleic acid that is complementary to a longer template. During replication, the primer may be extended, based on the template sequence, to produce a longer nucleic acid that is a complimentary copy of the template. Extension may occur by successive addition of individual nucleotides (e.g., by the action of a polymerase) or by attachment of a block of nucleotides (e.g., by the action of a ligase joining a pair of primers), among others. A primer may be DNA, RNA, an analog thereof (e.g., an artificial nucleic acid), or any combination thereof. A primer may have any suitable length, such as at least about 10 to about 30 nucleotides, or about 15 to about 30 nucleotides, for example, about 15, 16, 17, 18, 19, 20, 21, 22, 23, 24, 25, 26, 27, 28, 29, or 30 nucleotides. Primers are typically synthesized chemically. Primers may be supplied as at least one pair of primers for amplification of at least one nucleic acid target. A pair of primers may be a forward primer and a reverse primer (i.e., a sense primer and an antisense primer) that collectively define the opposing ends (and thus the length) of a resulting amplicon.

In some embodiments, the system or kit provides a pair of primers that specifically detect a microorganism or cell of interest. In some embodiments, the microorganism is a bacterium. In some embodiments, the bacterium is from a genus selected from Actinomyces, Bacteroides, Bacillus, Bordetella, Campylobacter, Clostridium, Corynebacterium, Enterobacter, Enterococcus, Escherichia, Fusobacterium, Haemophilus, Helicobacter, Klebsiella, Legionella, Leptospira, Listeria, Mycobacterium, Mycoplasma, Neisseria, Pasteurella, Pseudomonas, Staphylococcus, Streptobacillus, Streptococcus, Treponema, Vibrio, and Yersinia. In some embodiments, the microorganism is a virus. In some embodiments, the virus is from a viral family selected from Retroviridae (for example, human immunodeficiency viruses, such as HIV-1 (also referred to as HTLV-III, LAV or HTLV-III/LAV, or HIV-III) and other isolates, such as HIV-LP); Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, Ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arenaviridae (hemorrhagic fever viruses); Reoviridae (for example, reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV)-1 and HSV-2, varicella zoster virus, cytomegalovirus (CMV), herpes viruses); Poxyiridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (for example, Hepatitis C); Norwalk and related viruses, and astroviruses)). In some embodiments, the microorganism is a fungus. In some embodiments, the microorganism is a yeast, such as Saccharomyces (e.g., Saccharomyces cerevisiae) or Candida (e.g., Candida albicans). In some embodiments, the cell of interest is a eukaryotic cell, such as a mammalian cell or a plant cell.

Some embodiments herein find use in the detection of RNA from viable cells and/or intact viruses. In such embodiments, compound described herein having a RAB moiety is used to bind RNA in non-viable cells and/or viruses (e.g., RNA viruses) with degraded capsids. Amplification of the remaining RNA (e.g., not crosslinked by the multifunctional compounds herein) reveals the RNA from viable cells and/or viruses with intact capsids. In some embodiments, the virus is an RNA virus, such as those of the order Nidovirales, Picornavirales, or Tymovirales. In some embodiments, the virus is an RNA virus, such as those of the families Arteriviridae, Coronaviridae, Mesoniviridae, Roniviridae, Dicistroviridae, Iflaviridae, Marnaviridae, Picornaviridae, Secoviridae, Alphaflexiviridae, Betaflexiviridae, Gammaflexiviridae, Tymoviridae, Caliciviridae, Flaviviridae, Togaviridae, etc. In some embodiments, the RNA virus is an important human pathogen, such as SARS-CoV-2, HIV-1 Rhinovirus, Hepatitis A virus, Yellow fever virus, West Nile virus, Hepatitis C virus, Dengue fever virus, Zika virus, etc.

The pair of primers can be designed to detect an amplicon of a particular length. For example, the amplicon may be about 200 base pairs to about 1000 base pairs, or about 400 base pairs to about 750 base pairs. For example, in some embodiments, the primers in the system or kit are for an amplicon of at least about 200 base pairs, at least about 300 base pairs, at least about 400 base pairs, at least about 500 base pairs, at least about 600 base pairs, at least about 700 base pairs, at least about 800 base pairs, at least about 900 base pairs, or at least about 1000 base pairs. In some embodiments, the primers in the system or kit are for an amplicon of about 200, 250 300, 350, 400, 450, 500, 550, 600, 650, 700, 750, 800, 850, 900, 950, or 1000 base pairs.

In some embodiments, the system or kit can also include one or more probes, or any nucleic acid connected to at least one label, such as at least one dye. A probe may be a sequence-specific binding partner for a nucleic acid target and/or amplicon. The probe may be designed to enable detection of target amplification based on fluorescence resonance energy transfer (FRET), including one or more nucleic acids connected to a pair of dyes that collectively exhibit fluorescence resonance energy transfer (FRET) when proximate one another. The pair of dyes may provide first and second emitters, or an emitter and a quencher, among others. Fluorescence emission from the pair of dyes changes when the dyes are separated from one another, such as by cleavage of the probe during primer extension (e.g., a 5′ nuclease assay, such as with a TAQMAN probe), or when the probe hybridizes to an amplicon (e.g., a molecular beacon probe). The nucleic acid portion of the probe may have any suitable structure or origin, for example, the portion may be a locked nucleic acid, a member of a universal probe library, or the like. In other cases, a probe and one of the primers of a primer pair may be combined in the same molecule. For example, the primer-probe molecule may include a primer sequence at its 3′ end and a molecular beacon-style probe at its 5′ end. With this arrangement, related primer-probe molecules labeled with different dyes can be used in a multiplexed assay with the same reverse primer to quantify target sequences differing by a single nucleotide (single nucleotide polymorphisms (SNPs)).

In some embodiments, the system or kit can also include one or more labels or reporter molecules. Exemplary dyes used for labeling are fluorescent dyes (fluorophores) and fluorescence quenchers. A reporter includes any compound or set of compounds that reports a condition such as the extent of a reaction. Exemplary reporters comprise at least one dye, such as a fluorescent dye or an energy transfer pair, and/or at least one oligonucleotide. Exemplary reporters for nucleic acid amplification assays may include a probe and/or an intercalating dye (e.g., SYBR Green, ethidium bromide, etc.).

In some embodiments, the system or kit further comprises a magnesium salt, such as magnesium chloride or magnesium sulfate. In some embodiments, the system or kit further comprises magnesium chloride.

In some embodiments, the system or kit further comprises a buffer. The buffer may be provided as a separate component, or one or more of the other system or kit components (e.g., the DNA polymerase) can be provided as a solution in the buffer. Exemplary buffers include tris(hydroxymethyl)aminomethane (Tris) buffers. The buffer can further comprise a salt, such as an ammonium salt (e.g., ammonium chloride or ammonium sulfate), and/or a potassium salt (e.g., potassium chloride). The buffer can be provided at a suitable pH, which may be particularly tailored to the DNA polymerase provided with the system or kit. In some embodiments, the buffer has a pH of about 7.5 to about 10, or about 8.0 to about 9.5.

In some embodiments, the system or kit further comprises a reverse transcriptase, which is used in an RT-PCR reaction to make a complementary DNA (cDNA) from RNA, such that the cDNA can then be amplified in an amplification reaction. RT-PCR reactions may be used for detecting a variety of RNA species, such as RNA from a virus. The system or kit may also further comprise other components for RT-PCR, such as a ribonuclease inhibitor to inhibit degradation of the target during cDNA synthesis.

In some embodiments, kits are provided that contain one or more or all of the components necessary, sufficient, or useful for practicing the methods described herein (e.g., a compound described herein, a DNA polymerase, and amplification reagents). In some embodiments, the kits comprise positive control reagents, negative control reagents, quantitation standard reagents, and internal amplification control reagents. In some embodiments, the kits comprise instructions, which may be written instructions or embodied in a computer readable media. Reagents within the kits may be housed in one or more containers (e.g., tubes), and the collection of kit components may be packaged in one or more boxes or other containers that facilitate shipment and storage of the kit.

### 4. METHODS OF USE

Embodiments of the present disclosure include methods of amplifying nucleic acid (e.g., DNA or RNA) from a sample, wherein the nucleic acid (e.g., DNA or RNA) is selectively amplified from viable cells and not from non-viable cells in the sample (i.e., amplification reactions such as viability PCR or RT-PCR reactions).

Generally, amplification reactions involve a process of replication or forming a copy (e.g., a direct copy and/or a complimentary copy) of a nucleic acid or a segment thereof. Replication reactions generally involve an enzyme, such as a polymerase and/or a ligase, among others. The nucleic acid and/or segment replicated is a template (and/or a target) for replication. The reactions also generally involve a process of amplification, or a reaction in which replication occurs repeatedly over time to form multiple copies of at least one segment of a template molecule. Amplification may generate an exponential or linear increase in the number of copies as amplification proceeds. Typical amplifications produce a greater than 1,000-fold increase in copy number and/or signal. Exemplary amplification reactions for the assays disclosed herein may include the polymerase chain reaction (PCR) or ligase chain reaction (LCR), each of which is driven by thermal cycling. In some embodiments, the reaction comprises a step of reverse transcribing RNA from the sample to cDNA. The cDNA is then amplified using the methods described herein. Thermal cycling generally involves cycles of heating and cooling a reaction mixture to perform successive rounds of denaturation (melting), annealing, and extension. Other exemplary amplification reactions include isothermal amplification methods, which use an enzyme (e.g., a DNA polymerase) having strand-displacement activity.

In some embodiments, the disclosure provides a method of detecting a viable microorganism or cell of interest in the sample, the method comprising: (a) contacting the sample with a compound described herein (i.e., a compound comprising a NAB moiety, a LDCD moiety, and a NAM moiety), or a salt thereof, to form a first mixture; (b) contacting the first mixture with an inactivating agent to form a second mixture; and (c) amplifying nucleic acids from the second mixture to produce a detectable signal, wherein the signal is indicative of the presence of a viable organism in the sample. In some embodiments, the NAB is a RAB moiety and amplifying the nucleic acids comprises reverse transcribing the RNA into cDNA followed by amplification of the cDNA.

In some embodiments, the disclosure provides a method of amplifying a nucleic acid from a sample, the method comprising: (a) contacting the sample with a compound described herein (i.e., a compound comprising a NAB moiety, a LDCD moiety, and a NAM moiety), or a salt thereof, to form a first mixture; (b) contacting the first mixture with an inactivating agent to form a second mixture; and (c) amplifying nucleic acids from the second mixture. In some embodiments, the NAB is a RAB moiety and amplifying the nucleic acids comprises reverse transcribing the RNA into cDNA followed by amplification of the cDNA.

In some embodiments, the disclosure provides a method of detecting a viable microorganism or cell of interest in the sample, the method comprising: (a) contacting the sample with a compound described herein (i.e., a compound comprising a NAB moiety, a LDCD moiety, and a NAM moiety), or a salt thereof, to form a first mixture; (b) removing the multifunctional compound described herein (i.e., a compound comprising a NAB moiety, a LDCD moiety, and a NAM moiety) from the first mixture to form a second mixture; and (c) amplifying nucleic acids from the second mixture to produce a detectable signal, wherein the signal is indicative of the presence of a viable organism in the sample. In some embodiments, the NAB is a RAB moiety and amplifying the nucleic acids comprises reverse transcribing the RNA into cDNA followed by amplification of the cDNA.

In some embodiments, the disclosure provides a method of removing nucleic acid from non-viable microorganisms or cells of interest from a sample, the method comprising: (a) contacting the sample with a compound described herein (i.e., a compound comprising a NAB moiety, a LDCD moiety, and an affinity moiety (and optionally a NAM moiety)), or a salt thereof, and allowing the multifunctional compound to bind nucleic acids from non-viable microorganisms or cells of interest to form a first mixture; (b) removing nucleic acid from non-viable microorganisms or cells of interest from the first mixture by contacting the first mixture with an affinity agent, allowing the affinity agent to bind to the affinity moiety, and removing the affinity agent from the first mixture to form a second mixture; and (c) amplifying nucleic acids from the second mixture to produce a detectable signal, wherein the signal is indicative of the presence of viable microorganisms or cells in the sample. In some embodiments, the NAB is a RAB moiety and amplifying the nucleic acids comprises reverse transcribing the RNA into cDNA followed by amplification of the cDNA. In some embodiments, the microorganism is an RNA virus.

In some embodiments, the disclosure provides a method of removing nucleic acid from non-viable microorganisms or cells of interest from a sample, the method comprising: (a) contacting the sample with a compound described herein (i.e., a compound comprising a NAB moiety, a LDCD moiety, and a conjugation moiety (and optionally a NAM moiety)), or a salt thereof, and allowing the multifunctional compound to bind nucleic acids from non-viable microorganisms or cells of interest to form a first mixture; (b) removing nucleic acid from non-viable microorganisms or cells of interest from the first mixture by contacting the first mixture with a conjugation agent, allowing the conjugation agent to bind to the conjugation moiety, and removing the conjugation agent from the first mixture to form a second mixture; and (c) amplifying nucleic acids from the second mixture to produce a detectable signal, wherein the signal is indicative of the presence of viable microorganisms or cells in the sample. In some embodiments, the NAB is a RAB moiety and amplifying the nucleic acids comprises reverse transcribing the RNA into cDNA followed by amplification of the cDNA. In some embodiments, the microorganism is an RNA virus.

As discussed herein, the multifunctional compounds disclosed herein do not require a photoactivation step in order to modify nucleic acids (e.g., DNA). This provides advantages to the viability amplification processes (e.g., PCR, RT-PCR, etc.), particularly in complex or turbid samples, as it can reduce sample-to-sample variability. Accordingly, in some embodiments, the methods described herein do not include a photoactivation step.

Furthermore, the multifunctional compounds described herein can modify nucleic acid (e.g., DNA, RNA) from non-viable cells in a sample, but the LDCD moiety prevents their entry into viable cells or viruses, or allows for degradation of the LDCD in viable cells or viruses. This allows selective labeling of nucleic acid (e.g., DNA, RNA) from non-viable cells in the sample without the need for a culturing step to increase the number of live cells in the sample. Accordingly, in some embodiments, the methods described herein do not include a culturing step.

The sample from which the microorganism or cell is detected and/or the nucleic acid is amplified can be any sample for which it would be desirable to amplify nucleic acids selectively from live cells, or to detect a viable microorganism or cell in the sample. In some embodiments, the sample can be obtained from a subject. For example, non-limiting examples of samples obtained from a subject can include skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues. The sample can also be processed, extracted, or fractionated from any of the foregoing. In other embodiments, the sample is an environmental sample, such as a sample collected from a natural environment (e.g., soil, a body of water, or outdoor air), or an artificial environment (e.g., a clean room, a hospital facility, a food production facility, a laboratory facility, a pharmaceutical facility, a spa, a cooling tower, or an air handling system). In some embodiments, the sample is a food product, a pharmaceutical product, a water sample, or a soil sample.

In some embodiments, the sample can be a sample suspected of containing a viable microorganism, for which it would be desirable to detect the presence of such viable microorganism. For example, in some embodiments, the sample is a sample suspected of containing a bacterium, such as a bacterium from a genus selected from Actinomyces, Bacteroides, Bacillus, Bordetella, Campylobacter, Clostridium, Corynebacterium, Enterobacter, Enterococcus, Escherichia, Fusobacterium, Haemophilus, Helicobacter, Klebsiella, Legionella, Leptospira, Listeria, Mycobacterium, Mycoplasma, Neisseria, Pasteurella, Pseudomonas, Staphylococcus, Streptobacillus, Streptococcus, Treponema, Vibrio, and Yersinia. For example, in certain embodiments, the bacterium is Escherichia coli (e.g., E. coli O157:H7), Legionella pneumophila, Listeria monocytogenes, Mycobacterium tuberculosis, Pseudomonas aeruginosa, Salmonella enterica, or Staphylococcus aureus (e.g., methicillin-resistant Staphylococcus aureus). In some embodiments, the sample is a sample suspected of containing a virus, such as a virus from a family selected from Retroviridae, Picornaviridae, Calciviridae, Flaviridae, Coronaviridae, Rhabdoviridae, Filoviridae, Paramyxoviridae, Orthomyxoviridae, Bungaviridae, Arenaviridae, Reoviridae, Birnaviridae, Hepadnaviridae, Parvoviridae, Papovaviridae, Adenoviridae, Herpesviridae, Poxyiridae, and Iridoviridae. In some embodiments, the sample is a sample suspected of containing a fungus, a yeast, or another type of eukaryotic cell such as a mammalian cell or a plant cell. In some embodiments, the sample is suspected of containing a virus. In some embodiments, the virus is an RNA virus, such as those of the order Nidovirales, Picornavirales, or Tymovirales. In some embodiments, the virus is an RNA virus, such as those of the families Arteriviridae, Coronaviridae, Mesoniviridae, Roniviridae, Dicistroviridae, Iflaviridae, Marnaviridae, Picornaviridae, Secoviridae, Alphaflexiviridae, Betaflexiviridae, Gammaflexiviridae, Tymoviridae, Caliciviridae, Flaviviridae, Togaviridae, etc. In some embodiments, the RNA virus is an important human pathogen, such as SARS-CoV-2, HIV-1 Rhinovirus, Hepatitis A virus, Yellow fever virus, West Nile virus, Hepatitis C virus, Dengue fever virus, Zika virus, etc.

Some methods disclosed herein include a step of contacting a sample with the multifunctional compound comprising the NAB moiety (e.g., RAB moiety, DAB moiety, etc.), the LDCD moiety, and the NAM moiety (and optionally an affinity or conjugation moiety), to form a first mixture. In some embodiments, methods herein comprise a step of contacting a sample with compound comprising a NAB moiety (e.g., RAB moiety, DAB moiety, etc.), an LDCD moiety, and an affinity or conjugation moiety (and optionally a NAM moiety). The contacting step can be conducted by adding the multifunctional compound to the sample, and incubating the sample for a period of time sufficient to allow the multifunctional compound to bind to and modify nucleic acids in the sample that are not present in viable cells or intact viruses (i.e., free nucleic acids or nucleic acids in non-viable cells or viruses with degraded capsids). The contacting step can be conducted for about 5 minutes to about 180 minutes, or about 60 minutes to about 120 minutes, e.g., about 5, 10, 15, 20, 25, 30, 35, 40, 45, 50, 55, 60, 65, 70, 75, 80, 85, 90, 95, 100, 105, 110, 115, or 120 minutes. In some embodiments, step (a) comprises contacting the sample with the multifunctional compound for about 90 minutes.

The concentration of the multifunctional compound in the first mixture can range from about 1 micromolar to about 200 micromolar, or about 5 micromolar to about 100 micromolar, e.g., about 5, 10, 15, 20, 25, 30, 35, 40, 45, 50, 55, 60, 65, 70, 75, 80, 85, 90, 95, or about 100 micromolar.

Step (a) can be conducted at any suitable temperature. For example, step (a) can be conducted at room temperature (e.g., about 20-25° C.), or at about 37° C.

In some embodiments, the contacting step of step (a) is conducted by adding a composition comprising the multifunctional compound to the sample, for example, a composition comprising the multifunctional compound and a solvent. In some embodiments, the solvent is water. In some embodiments, the solvent is an organic solvent such as dimethylsulfoxide (DMSO). Use of a solvent such as DMSO may have particular advantages in samples comprising Gram-negative bacteria, as discussed above.

In certain embodiments, step (b) of some methods herein comprises removing, inactivating, or otherwise neutralizing the multifunctional compound in the first mixture to form a second mixture. In some embodiments, step (b) comprises contacting the first mixture with an inactivating agent to form a second mixture. This step serves to inactivate the NAM moiety in any excess compound in the first mixture, which renders it unable to modify any further nucleic acids and ensure optimal performance in the subsequent amplification reaction. However, in some embodiments, NAM moieties that have modified nucleic acid in the sample are not affected by this step. For example, if excess compound remained after step (a), then a subsequent lysis of viable cells in the sample would be modified by the excess compound and would not be available for the amplification reaction. In such embodiments, the inactivating agent will depend on the particular NAM moiety in the multifunctional compound. For example, when the NAM moiety is a nitrogen mustard moiety, the inactivating agent can be a nucleophilic compound that can effectively displace the chloride groups, such as an amine or a thiol. Accordingly, in some embodiments, the inactivating agent is a compound comprising a thiol, such as cysteine, glutathione, or DTT. In some embodiments, the inactivating agent is an amine-containing compound, such as an amine-containing buffer. Examples of amine-containing buffers include tris(hydroxymethyl)aminomethane (Tris) and triethanolamine-containing buffers. In some embodiments, the inactivating agent is a dNTP or mixture of dNTPs. In some embodiments, the inactivating agent is a nucleotide, such as guanine. In other embodiments, the inactivating step serves to inhibit the NAB (e.g., DAB or RAB) moiety, thereby preventing the multifunctional compounds from binding to and modifying the DNA. In such embodiments, the inactivating agent will depend on the particular NAB moiety in the multifunctional compound yet other embodiments, the multifunctional compound is physically removed from the first mixture to form the second mixture, for example, by washing the sample. In embodiments in which the multifunctional compound comprises an affinity or conjugation moiety, binding of the multifunctional compound in the first mixture (free or bound to nucleic acid) by an affinity or conjugation agent, and removing the agent from the mixture, provides and efficient step of removing unbound/unreacted multifunctional compound from the mixture.

In some embodiments, step (c) comprises amplifying nucleic acids from the second mixture to produce a detectable signal, wherein the signal is indicative of the presence of a viable organism in the sample. The amplification reaction can be performed in a variety of ways. For example, in some embodiments (e.g., when the target nucleic acid is DNA), the amplification step (c) comprises steps of: (i) lysing cells and/or viruses in the second mixture to form a lysed sample; (ii) adding a DNA polymerase and amplification reagents to the lysed sample to form a mixture; and (iii) subjecting the mixture to a thermal cycling protocol to amplify the nucleic acid from the sample. In other embodiments, the amplification step (c) comprises steps of: (i) lysing cells in the second mixture to form a lysed sample; (ii) adding a DNA polymerase and amplification reagents to the lysed sample to form a mixture; and (iii) subjecting the mixture to an isothermal amplification reaction to amplify the nucleic acid from the sample.

In some embodiments, step (c) comprises reverse transcribing RNA from the second mixture to produce cDNA, and then amplifying the cDNA to yield a detectable signal, wherein the signal is indicative of the presence of a viable organism (e.g., RNA virus) in the sample. The reverse transcription/amplification reaction can be performed in a variety of ways. For example, in some embodiments, step (c) comprises steps of: (i) lysing cells and/or viruses in the second mixture to form a lysed sample; (ii) adding a reverse transcriptase and amplification reagents to the lysed sample to form a mixture; (iii) subjecting the mixture to a thermal cycling protocol to reverse transcribe the RNA to cDNA; (iv) adding a DNA polymerase and amplification reagents to the lysed sample to form a mixture; and (v) subjecting the mixture to a thermal cycling protocol to amplify the nucleic acid from the sample. In some embodiments, steps (ii) through (iv) are performed concurrently.

In some embodiments, a lysis step exposes nucleic acid (e.g., RNA and/or DNA) from the viable cells or viruses in the sample so that it can be amplified in the amplification reaction (and/or reverse transcribed). Any suitable method of cell lysis can be used in the methods, e.g., chemical lysis, electrochemical lysis, acoustic lysis (i.e., sonication), mechanical lysis, or heat lysis.

Methods herein may comprise additional steps and/or the order of steps may be modified according to the particular application. For example, in some embodiments, the method further comprises a step of removing contaminants and/or cellular debris from the lysed sample prior to adding the amplification enzymes and reagents. For example, this step may involve purifying nucleic acid (e.g., RNA and/or DNA) from the sample to generate a purified nucleic acid (e.g., RNA and/or DNA) sample to which the amplification enzymes and reagents can be added. The nucleic acid (e.g., RNA and/or DNA) can be purified by any conventional means, for example, using organic extraction followed by ethanol precipitation, a salt-based precipitation method, magnetic particle-based isolation, or stationary phase adsorption methods. In certain embodiments, an affinity moiety and/or conjugation moiety on the multifunctional compound is used for purification/isolation, by contacting the nucleic acid bound by the multifunctional compound with an affinity agent or conjugation agent. In some embodiments, the affinity agent or conjugation agent is bound to a solid surface (e.g., bead, column, plate, etc.). In some embodiments, the unbound components of the sample (e.g., nucleic acids in viable cells or viruses that is not bound/modified by the multifunctional compound) are separated from the agent-bound nucleic acids by known methods.

In some embodiments in which the nucleic acid being analyzed/manipulated in the sample is DNA, the DNA is amplified in an amplification reaction such as PCR. Generally, PCR includes any nucleic acid amplification reaction that relies on alternating cycles of heating and cooling (i.e., thermal cycling) to achieve successive rounds of replication. PCR may be performed by thermal cycling between two or more temperature set points, such as a higher melting (denaturation) temperature and a lower annealing/extension temperature, or among three or more temperature set points, such as a higher melting temperature, a lower annealing temperature, and an intermediate extension temperature, among others. PCR generally produces an exponential increase in the amount of a product amplicon over successive cycles.

In some embodiments in which the nucleic acid being analyzed/manipulated in the sample is RNA, the RNA is reverse transcribed using a reverse transcriptase enzyme and amplification reagents in a RT-PCR reaction to first produce cDNA from the RNA, and then the cDNA can be amplified using the methods and reagents described herein. Any amplification techniques or reagents described herein for the amplification of DNA can be employed to amplify cDNA reverse transcribed from RNA.

Any suitable PCR methodology or combination of methodologies may be utilized in the embodiments disclosed herein such as allele-specific PCR, assembly PCR, asymmetric PCR, digital PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, RT-PCR, single-cell PCR, solid-phase PCR, thermal asymmetric interlaced PCR, touchdown PCR, universal fast walking PCR, or any combination thereof, among others.

In some embodiments, the nucleic acid (e.g., DNA or RNA) is amplified in an isothermal amplification reaction, which generally includes any nucleic acid amplification reaction that relies on an enzyme, rather than thermal denaturation, to directly unwind the DNA double helix in order to synthesize complementary strands. Examples of isothermal amplification methods include loop-mediated isothermal amplification (LAMP), helicase-dependent amplification (HAD), rolling circle amplification (RCA), multiple displacement amplification (MDA), nucleic acid sequence-based amplification (NASBA), whole genome amplification (WGA), and recombinase polymerase amplification (RPA).

The amplification can be conducted by adding amplification reagents and a DNA polymerase to form a mixture. DNA polymerases that can be used in accordance with these embodiments include, but are not limited to, any polymerase capable of replicating a DNA molecule. In some embodiments, the DNA polymerase is a thermostable polymerase, which is especially useful in PCR applications. In some embodiments, the thermostable DNA polymerase is selected from Taq, Tbr, Tfi, Tfl, KOD, Tru, Tth, Tli, Tac, Tne, Tma, Pfu, Pho, Pwo, ES4, Bca, Bst, Sac, Sso, Poc, Pab, and Mth, or a mutant, variant, or derivative of any thereof. In some embodiments, the DNA polymerase is Taq polymerase. In some embodiments, the DNA polymerase is a polymerase having strand displacement activity, which is useful in isothermal amplification applications. In some embodiments, the DNA polymerase having strand displacement activity is selected from Bsm, Bst, Bsu, and phi29 DNA polymerases.

The one or more additional amplification reagents can be selected from at least one primer or at least one pair of primers for amplification of a nucleic acid target, at least one probe and/or dye to enable detection of amplification, a buffer, a ligase, a reverse transcriptase, a detergent (e.g., non-ionic detergents), nucleotides (dNTPs and/or NTPs), a magnesium salt (e.g., magnesium chloride), or any combination thereof, among other amplification reagents that would be recognized by one of ordinary skill in the art based on the present disclosure. For example, in some embodiments, the amplification reagents comprise at least one primer, deoxynucleotide triphosphates (dNTPs), a buffer, and a magnesium salt.

In some embodiments, the amplification reagents include one or more primers. A primer may be DNA, RNA, an analog thereof (e.g., an artificial nucleic acid), or any combination thereof. A primer may have any suitable length, such as at least about 10 to about 30 nucleotides, or about 15 to about 30 nucleotides, for example, about 15, 16, 17, 18, 19, 20, 21, 22, 23, 24, 25, 26, 27, 28, 29, or 30 nucleotides. Primers are typically synthesized chemically. Primers may be supplied as at least one pair of primers for amplification of at least one nucleic acid target. A pair of primers may be a forward primer and a reverse primer (i.e., a sense primer and an antisense primer) that collectively define the opposing ends (and thus the length) of a resulting amplicon.

In some embodiments, the amplification reagents include a pair of primers that specifically detect an organism or cell type of interest. In some embodiments, the organism is a microorganism. In some embodiments, the microorganism is a bacterium. In some embodiments, the bacterium is from a genus selected from Actinomyces, Bacteroides, Bacillus, Bordetella, Campylobacter, Clostridium, Corynebacterium, Enterobacter, Enterococcus, Escherichia, Fusobacterium, Haemophilus, Helicobacter, Klebsiella, Legionella, Leptospira, Listeria, Mycobacterium, Mycoplasma, Neisseria, Pasteurella, Pseudomonas, Staphylococcus, Streptobacillus, Streptococcus, Treponema, Vibrio, and Yersinia. In some embodiments, the microorganism is a virus. In some embodiments, the virus is from a family selected from Retroviridae, Picornaviridae, Calciviridae, Flaviridae, Coronaviridae, Rhabdoviridae, Filoviridae, Paramyxoviridae, Orthomyxoviridae, Bungaviridae, Arenaviridae, Reoviridae, Birnaviridae, Hepadnaviridae, Parvoviridae, Papovaviridae, Adenoviridae, Herpesviridae, Poxyiridae, and Iridoviridae. In some embodiments, the virus is an RNA virus, such as those of the order Nidovirales, Picornavirales, or Tymovirales. In some embodiments, the virus is an RNA virus, such as those of the families Arteriviridae, Coronaviridae, Mesoniviridae, Roniviridae, Dicistroviridae, Iflaviridae, Marnaviridae, Picornaviridae, Secoviridae, Alphaflexiviridae, Betaflexiviridae, Gammaflexiviridae, Tymoviridae, Caliciviridae, Flaviviridae, Togaviridae, etc. In some embodiments, the RNA virus is an important human pathogen, such as SARS-CoV-2, HIV-1 Rhinovirus, Hepatitis A virus, Yellow fever virus, West Nile virus, Hepatitis C virus, Dengue fever virus, Zika virus, etc. In some embodiments, the microorganism is a fungus. In some embodiments, the microorganism is a yeast. In some embodiments, the cell type of interest is a plant cell or mammalian cell.

The pair of primers can be designed to detect an amplicon of a particular length. For example, the amplicon may be about 200 base pairs to about 1000 base pairs, or about 400 base pairs to about 750 base pairs. For example, in some embodiments, the primers are for an amplicon of at least about 200 base pairs, at least about 300 base pairs, at least about 400 base pairs, at least about 500 base pairs, at least about 600 base pairs, at least about 700 base pairs, at least about 800 base pairs, at least about 900 base pairs, or at least about 1000 base pairs. In some embodiments, the primers are for an amplicon of about 200, 250 300, 350, 400, 450, 500, 550, 600, 650, 700, 750, 800, 850, 900, 950, or 1000 base pairs.

In some embodiments, the amplification reagents comprise a magnesium salt, such as magnesium chloride or magnesium sulfate. In some embodiments, the amplification reagents comprise magnesium chloride.

In some embodiments, the amplification reagents comprise a buffer. Exemplary buffers include tris(hydroxymethyl)aminomethane (Tris) buffers. The buffer can further comprise a salt, such as an ammonium salt (e.g., ammonium chloride or ammonium sulfate), and/or a potassium salt (e.g., potassium chloride). The pH of the buffer may be particularly tailored to the DNA polymerase being used in the amplification reaction. In some embodiments, the buffer has a pH of about 7.5 to about 10, or about 8.0 to about 9.5.

In some embodiments, such as embodiments of the methods to detect a viable microorganism in a sample, the amplification reagents comprise one or more probes, or any nucleic acid connected to at least one label, such as at least one dye. A probe may be a sequence-specific binding partner for a nucleic acid target and/or amplicon. The probe may be designed to enable detection of target amplification based on fluorescence resonance energy transfer (FRET), including one or more nucleic acids connected to a pair of dyes that collectively exhibit fluorescence resonance energy transfer (FRET) when proximate one another. The pair of dyes may provide first and second emitters, or an emitter and a quencher, among others. Fluorescence emission from the pair of dyes changes when the dyes are separated from one another, such as by cleavage of the probe during primer extension (e.g., a 5′ nuclease assay, such as with a TAQMAN probe), or when the probe hybridizes to an amplicon (e.g., a molecular beacon probe). The nucleic acid portion of the probe may have any suitable structure or origin, for example, the portion may be a locked nucleic acid, a member of a universal probe library, or the like. In other cases, a probe and one of the primers of a primer pair may be combined in the same molecule. For example, the primer-probe molecule may include a primer sequence at its 3′ end and a molecular beacon-style probe at its 5′ end. With this arrangement, related primer-probe molecules labeled with different dyes can be used in a multiplexed assay with the same reverse primer to quantify target sequences differing by a single nucleotide (single nucleotide polymorphisms (SNPs)).

In some embodiments, the amplification reagents also include one or more labels or reporter molecules. Exemplary dyes used for labeling are fluorescent dyes (fluorophores) and fluorescence quenchers. A reporter includes any compound or set of compounds that reports a condition such as the extent of a reaction. Exemplary reporters comprise at least one dye, such as a fluorescent dye or an energy transfer pair, and/or at least one oligonucleotide. Exemplary reporters for nucleic acid amplification assays may include a probe and/or an intercalating dye (e.g., SYBR Green, ethidium bromide, etc.).

In accordance with the embodiments provided herein, concentrations of the amplification reagents described above can vary, depending on specific reaction conditions and reagents used, as well as the desired target to be amplified. One of skill in the art would readily recognize that any specific concentrations or concentration ranges provided herein for any amplification reagents, including concentration ranges pertaining to the multifunctional compounds of the present disclosure, will vary depending on the specific reaction conditions and reagents used and are not meant to be limiting.

In some embodiments, such as those involving PCR, after adding the DNA polymerase and the amplification reagents to form the mixture, the method further comprises heating the mixture to a temperature of at least 90° C. to activate the DNA polymerase prior to subjecting the mixture to the thermal cycling protocol. For example, the DNA polymerase may initially be unreactive at ambient temperature, via inhibition through antibody interaction or other modification. An initial step activates the DNA polymerization by causing dissociation from the inhibitor. In this step, the mixture can be subjected to a temperature of at least 90° C., e.g., about 90° C. to about 96° C., e.g., about 90° C., about 91° C., about 92° C., about 93° C., about 94° C., about 95° C., or about 96° C. This heating step can be conducted for about 1 minute to about 10 minutes, or about 2 minutes to about 5 minutes, e.g., about 1, 2, 3, 4, 5, 6, 7, 8, 9, or 10 minutes.

In some embodiments, the nucleic acid is amplified using a thermal cycling protocol. In some embodiments, the thermal cycling protocol comprises:
(1) a denaturation step comprising subjecting the mixture to a temperature of about 90-96° C.;
(2) an annealing step comprising subjecting the mixture to a temperature of about 45-68° C.; and
(3) an extension step comprising subjecting the mixture to a temperature of about 50-72° C.;

wherein the sequence of steps (1)-(3) is repeated about 10 or more times in succession.

The denaturation step comprises subjecting the mixture to a temperature of about 90-96° C. to denature the double-stranded DNA and allow for subsequent annealing of the primers. For example, the mixture can be subjected to a temperature of about 90° C. to about 96° C., e.g., about 90° C., about 91° C., about 92° C., about 93° C., about 94° C., about 95° C., or about 96° C. This step can be conducted for about 15 seconds to about 60 seconds, e.g., about 15 seconds, about 30 seconds, about 45 seconds, or about 60 seconds.

The annealing step comprises subjecting the mixture to a temperature of about 45-68° C. to allow the primers to bind to the complementary sequence on the denatured DNA. The optimal annealing temperature will depend on the particular primers being used, and is typically a temperature that is about 5° C. lower than the primer melting temperature (Tm). For example, the mixture can be subjected to a temperature of about 45° C. to about 68° C., e.g., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., or about 68° C. The annealing step can be conducted for about 15 seconds to about 60 seconds, e.g., about 15 seconds, about 30 seconds, about 45 seconds, or about 60 seconds.

The extension step comprises subjecting the mixture to a temperature of about 50-72° C. to allow the DNA polymerase to extend the DNA strands starting from the annealed primers. The extension temperature will depend on the particular DNA polymerase being used, and the extension time will depend on the length of the amplicon. A typical extension temperature is about 50° C. to about 72° C., e.g., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., or about 72° C. A typical extension time is about 1 minute per kilobase of DNA. For example, the extension step can be conducted for about 15 seconds to about 2 minutes, e.g., about 15 seconds, about 30 seconds, about 45 seconds, about 60 seconds, about 75 seconds, about 90 seconds, about 105 seconds, or about 120 seconds.

The sequence of denaturation, annealing, and extension steps can be repeated about 10 or more times in succession, e.g., about 10, 15, 20, 25, 30, 35, 40, 45, or 50 times in succession.

In other embodiments, the thermal cycling protocol may comprise only two steps, a denaturation step and a combined annealing/extension step. For example, in some embodiments the thermal cycling protocol comprises:
(1a) a denaturation step comprising subjecting the mixture to a temperature of 90-96° C.;
(2a) an annealing/extension step comprising subjecting the mixture to a temperature of 45-70° C.; and

wherein the sequence of steps (1a)-(2a) is repeated 10 or more times in succession.

In these embodiments, the annealing/extension step comprises subjecting the mixture to a temperature of about 45° C. to about 70° C., e.g., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C. The annealing/extension step can be conducted for about 15 seconds to about 2 minutes, e.g., about 15 seconds, about 30 seconds, about 45 seconds, about 60 seconds, about 75 seconds, about 90 seconds, about 105 seconds, or about 120 seconds.

The sequence of denaturation and annealing/extension steps can be repeated about 10 or more times in succession, e.g., about 10, 15, 20, 25, 30, 35, 40, 45, or 50 times in succession.

In some embodiments, the nucleic acid is amplified using an isothermal amplification protocol.

In methods of detecting a viable microorganism or cell in the sample, the method produces a detectable signal, wherein the signal is indicative of the presence of a viable organism or cell in the sample. In some embodiments of such methods, the method further comprises detecting the detectable signal from the sample. The specific detection step will depend on the probe and/or dye used in the method. Any suitable detection method can be used, such as photochemical, biochemical, spectroscopic, immunochemical, electrical, optical, or chemical means. In some embodiments, the detection step is a fluorescence detection step. For example, in some embodiments, the amplified products can be directly detected using fluorescence. In some embodiments, a fluorescent probe for the amplified products can be detected using fluorescence.

In some embodiments, the detection is performed using a spectrophotometric thermal cycler. Such thermal cyclers are commercially available from, for example, Agilent (e.g., AriaDx and AriaMx instruments), Applied Biosystems (e.g., QuantStudio® systems), Bio-Rad (e.g., CFX systems), Cepheid (e.g., SmartCycler®), Roche (e.g., LightCycler® systems), and Stratagene (e.g., Mx3005p).

In some embodiments, the disclosure provides a method of removing nucleic acids from a sample, the method comprising contacting the sample with a compound described herein (i.e., a compound comprising a NAB moiety, a LDCD moiety, and a NAM moiety). In some embodiments of such methods, the multifunctional compound is immobilized on a solid support, such as a bead, a resin, or a membrane. Such methods can be used with any sample for which it would be desirable to remove free nucleic acids. For example, certain reagents or solutions intended for use as human injectables may be treated with the multifunctional compounds described herein to remove free DNA.

### 5. EXAMPLES

It will be readily apparent to those skilled in the art that other suitable modifications and adaptations of the methods of the present disclosure described herein are readily applicable and appreciable and may be made using suitable equivalents without departing from the scope of the present disclosure or the aspects and embodiments disclosed herein. The disclosure will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

### Example 1

### Viability PCR Workflow

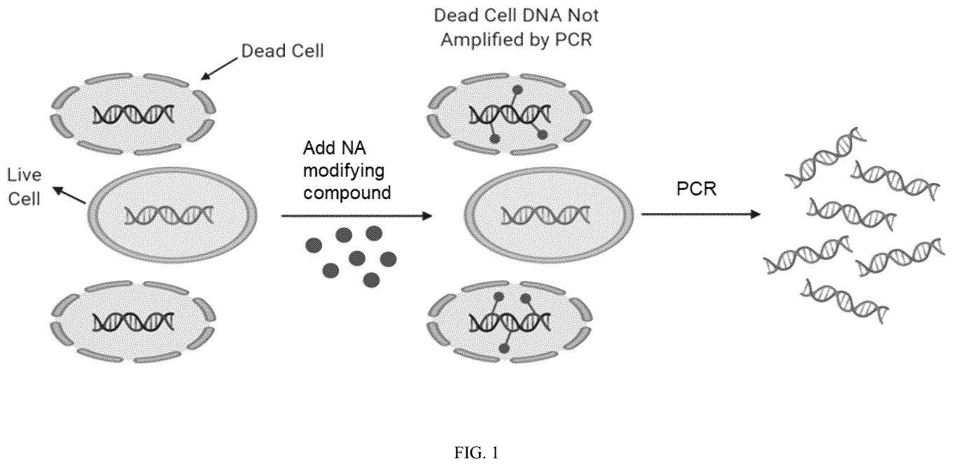

A diagram of the viability PCR (vPCR) workflow described in this application is shown in FIG. 1. In this procedure, live-cell impermeable nucleic acid-modifying compounds are incubated with a mixture of bacterial cells, entering only dead/dying cells due to the permeability of compromised cell membranes. As a result, only nucleic acid from non-viable cells is covalently crosslinked, preventing subsequent amplification via PCR. Therefore, the signal observed in a vPCR detection assay is derived exclusively from viable cells. Dead cells are notated by perforated grey membranes and live cells are notated by intact purple membranes.

### Example 2

### Compound Syntheses

Abbreviations used in this example include the following: Ac is acetyl; ACN is acetonitrile; DIPEA is N,N-diisopropylethylamine; DMF is N,N-dimethylformamide; HATU is (1-[bis(dimethylamino)methylene]-1H-1,2,3-triazolo[4,5-b]pyridinium 3-oxide hexafluorophosphate; HPLC is high performance liquid chromatography; RT is room temperature; TFA is trifluoroacetic acid; TIPS is triisopropylsilane; TMS is trimethylsilyl; and TSTU is N,N,N′,N′-tetramethyl-O—(N-succinimidyl)uronium tetrafluoroborate.

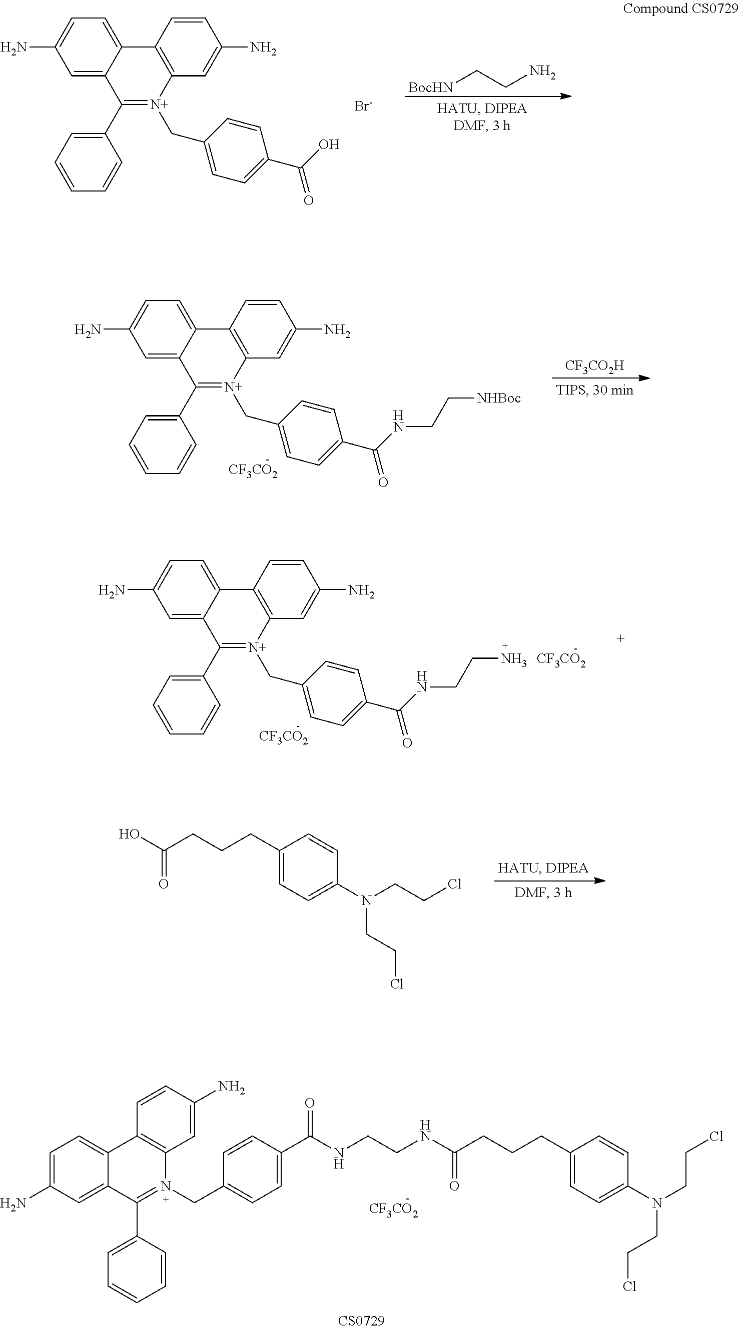

Step 1: 3,8-diamino-5-(4-carboxybenzyl)-6-phenylphenanthridin-5-ium bromide (50 mg, 0.1 mmol, 1.0 equiv) was added to a solution of tert-butyl (2-aminoethyl)carbamate (24 mg, 0.15 mmol, 1.5 equiv), DIPEA (39 mg, 0.3 mmol, 3.0 equiv) and HATU (57 mg, 0.15 mmol, 1.5 equiv) in DMF (2 mL). The solution was stirred at RT for 3 h and quenched by addition of 2 mL of CH₃CN/0.1% TFA in H₂O (1/1). The mixture was then purified by reverse phase HPLC to afford 3,8-diamino-5-(4-((2-((tert-butoxycarbonyl)amino)-ethyl)carbamoyl)benzyl)-6-phenylphenanthridin-5-ium 2,2,2-trifluoroacetate.

Step 2: 3,8-diamino-5-(4-((2-((tert-butoxycarbonyl)amino)-ethyl)carbamoyl)benzyl)-6-phenylphenanthri-din-5-ium 2,2,2-trifluoroacetate (67.5 mg, 0.1 mmol, 1.0 equiv) was dissolved in TFA (1 mL) and TIPS (0.1 mL) and stirred for 30 min. The reaction was concentrated in vacuo and purified by reverse phase HPLC to afford 5-(4-((2-(14-azaneyl)ethyl)carbamoyl)benzyl)-3,8-diamino-6-phenylphenanthridin-5-ium 2,2,2-trifluoroacetate.

Step 3: 5-(4-((2-(14-azaneyl)ethyl)carbamoyl)benzyl)-3,8-diamino-6-phenylphenanthridin-5-ium 2,2,2-trifluoroacetate (69 mg, 0.1 mmol, 1.0 equiv) was added to a solution of 4-(4-(bis(2-chloroethyl)amino)-phenyl)butanoic acid (33 mg, 0.11 mmol, 1.1 equiv), HATU (42 mg, 0.11 mmol, 1.1 equiv) and DIPEA (39 mg, 0.3 mmol, 3.0 equiv) in DMF (2 mL). The solution was stirred at RT for 3 h and quenched by addition of 2 mL of CH₃CN/0.1% TFA in H₂O (1/1). The mixture was then purified by reverse phase HPLC to afford 3,8-diamino-5-(4-((2-(4-(4-(bis(2-chloroethyl)amino)phenyl)butanamido)ethyl)carbamoyl)-benzyl)-6-phenylphenanthridin-5-ium 2,2,2-trifluoroacetate (CS0729).

Compounds CS0727, CS0775, CS0776, CS0777, CS0935, and CS0942 were synthesized analogously using the above procedure; structures and characterization data are shown in Table 1.

TABLE 1

| | | MS |
|---|---|---|
| Compound | Structure | (M+) |
| CS0729 | 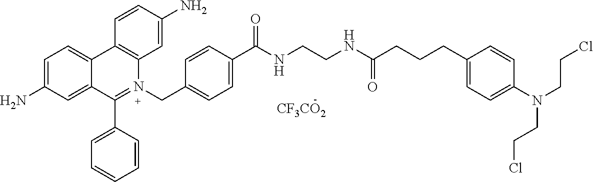 | 747.3 |
| CS0727 | 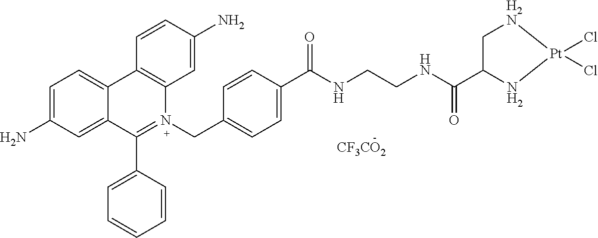 | 813.2 |
| CS0775 | | 403.7 |
| CS0776 | | 442.6 |
| CS0777 | 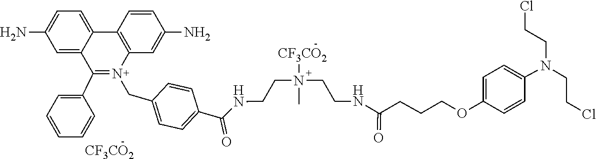 | 417.7 |
| CS0935 | 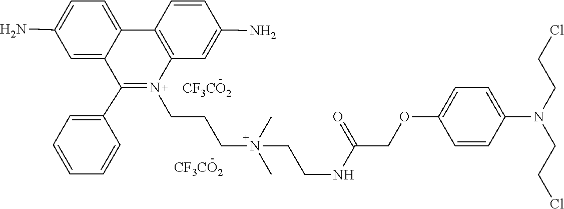 | 344.2 |
| CS0942 | 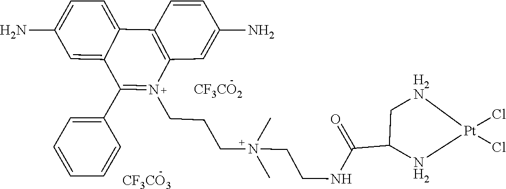 | 383.1 |

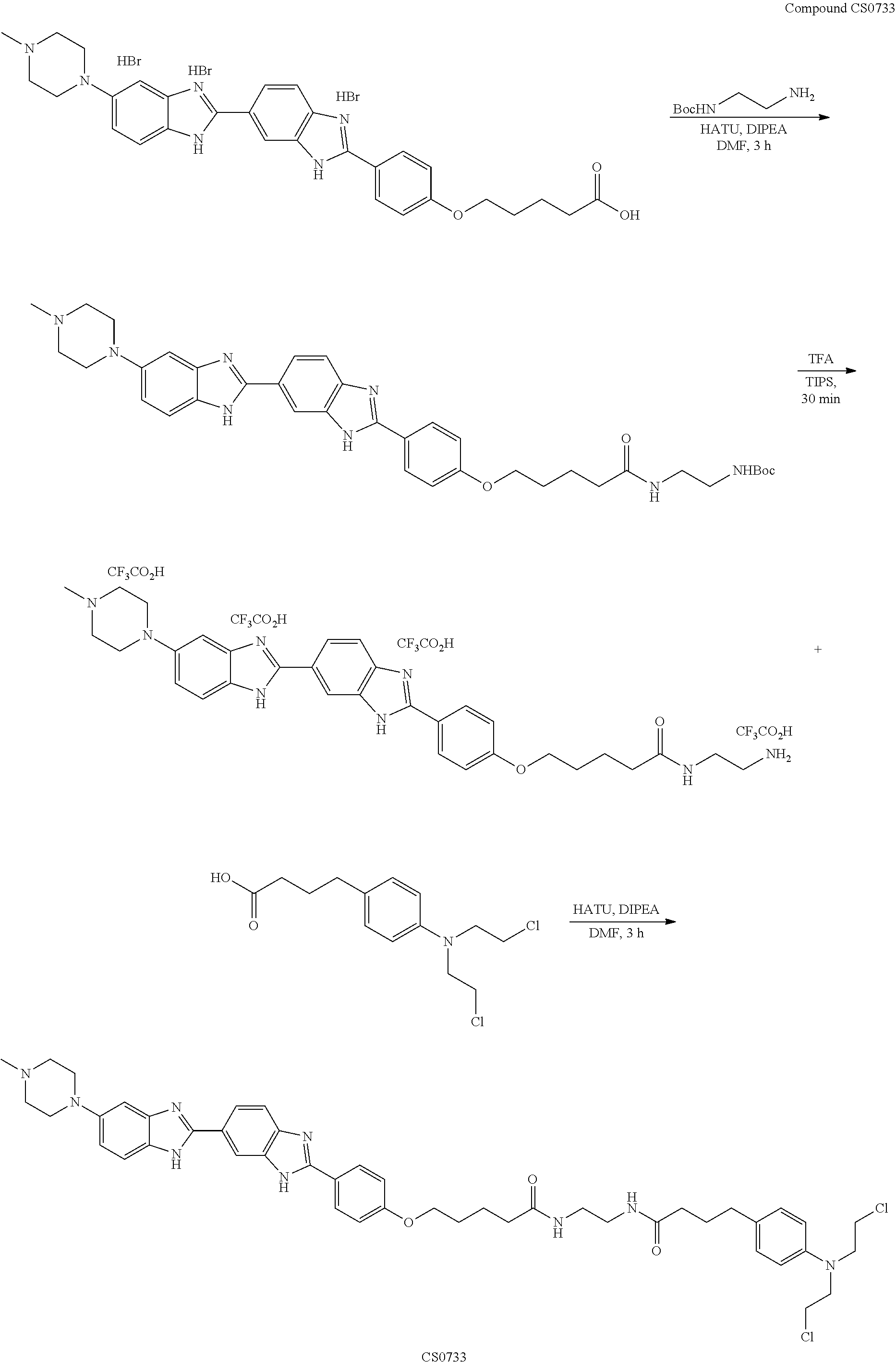

Step 1: 5-(4-(5-(4-methylpiperazin-1-yl)-1H,3′H-[2,5′-bibenzo[d]imidazol]-2′-yl)phenoxy)pentanoic acid trihydrobromide (76.6 mg, 0.1 mmol, 1.0 equiv) was added to a solution of tert-butyl (2-aminoethyl)carbamate (24 mg, 0.15 mmol, 1.5 equiv), DIPEA (78 mg, 0.6 mmol, 6.0 equiv) and HATU (57 mg, 0.15 mmol, 1.5 equiv) in DMF (2 mL). The solution was stirred at RT for 3 h and quenched by addition of 2 mL of CH₃CN/0.1% TFA in H₂O (1/1). The mixture was then purified via silica gel purification to afford tert-butyl (2-(5-(4-(5-(4-methylpiperazin-1-yl)-1H,3′H-[2,5′-bibenzo[d]imidazol]-2′-yl)phenoxy)pentanamido)ethyl)carbamate.

Step 2: tert-butyl (2-(5-(4-(5-(4-methylpiperazin-1-yl)-1H,3′H-[2,5′-bibenzo[d]imidazol]-2′-yl)phenoxy)-pentanamido)ethyl)carbamate (67 mg, 0.1 mmol, 1.0 equiv) was dissolved in TFA (1 mL) and TIPS (0.1 mL) and stirred for 30 min. The reaction was concentrated in vacuo and purified by reverse phase HPLC to afford N-(2-aminoethyl)-5-(4-(5-(4-methylpiperazin-1-yl)-1H,3′H-[2,5′-bibenzo[d]imidazol]-2′-yl)phenoxy)pentanamide tetrakis(2,2,2-trifluoroacetate).

Step 3: N-(2-aminoethyl)-5-(4-(5-(4-methylpiperazin-1-yl)-1H,3′H-[2,5′-bibenzo[d]imidazol]-2′-yl)phen-oxy)pentanamide tetrakis(2,2,2-trifluoroacetate) (101 mg, 0.1 mmol, 1.0 equiv) was added to a solution of 4-(4-(bis(2-chloroethyl)amino)-phenyl)butanoic acid (33 mg, 0.11 mmol, 1.1 equiv), HATU (42 mg, 0.11 mmol, 1.1 equiv) and DIPEA (117 mg, 0.9 mmol, 9.0 equiv) in DMF (2 mL). The solution was stirred at RT for 3 h and concentrated in vacuo. The mixture was then purified on normal phase silica gel to afford (CS0733).

Compound CS0717 was synthesized analogously using the above procedure; structures and characterization data are shown in Table 2.

TABLE 2

| | | MS |
|---|---|---|
| Compound | Structure | (M+) |
| CS0733 | 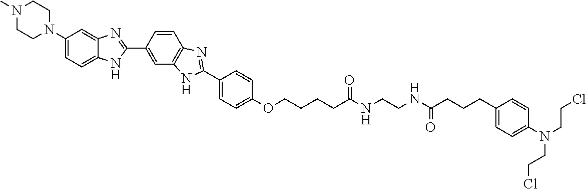 [SMILES: CN1CCN(c2ccc3[nH]c(-c4ccc5nc(-c6ccc(OCCCCC(=O)NCCNC(=O)CCCc7ccc(N(CCCl)CCCl)cc7)cc6)[nH]c5c4)nc3c2)CC1] | 852.9 |
| CS0717 | 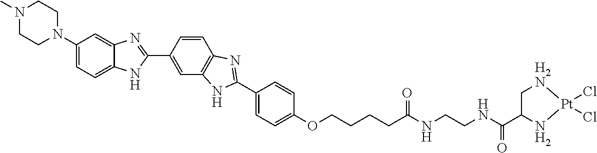 | 813.2 |

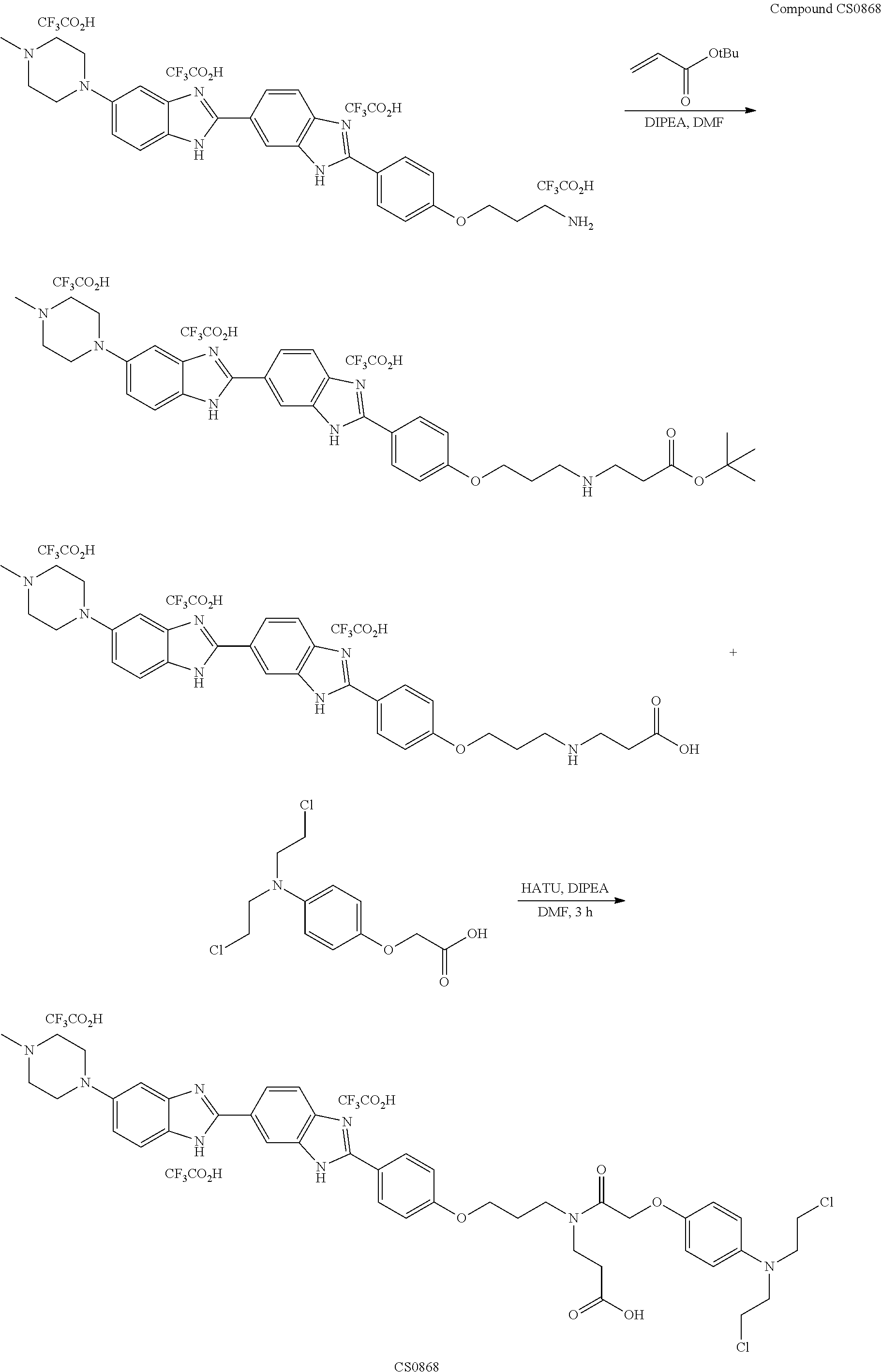

Step 1: 3-(4-(5-(4-methylpiperazin-1-yl)-1H,3′H-[2,5′-bibenzo[d]imidazol]-2′-yl)phenoxy)propan-1-amine tetrakis(2,2,2-trifluoroacetate) (93.7 mg, 0.1 mmol, 1.0 equiv) in DMF (1 mL) was added to a solution of tert-butyl acrylate (39 mg, 0.3 mmol, 3.0 equiv) and DIPEA (65 mg, 0.5 mmol, 5.0 equiv) in DMF (1 mL). The solution was stirred at RT for 48 h. The product was isolated using reverse phase HPLC to afford tert-butyl 3-((3-(4-(5-(4-methylpiperazin-1-yl)-1H,3′H-[2,5′-bibenzo [d]imidazol]-2′-yl)phenoxy)propyl)-ami-no)propanoate tris(2,2,2-trifluoroacetate).

Step 2: tert-butyl 3-((3-(4-(5-(4-methylpiperazin-1-yl)-1H,3′H-[2,5′-bibenzo[d]imidazol]-2′-yl)phenoxy)-propyl)-amino)propanoate tris(2,2,2-trifluoroacetate) (95 mg, 0.1 mmol, 1.0 equiv) was dissolved in TFA (1 mL) and TIPS (0.1 mL) and stirred for 30 min. The reaction was concentrated in vacuo and purified by reverse phase HPLC to afford 2,2,2-trifluoroacetic acid compound with 3-((3-(4-(5-(4-methylpiperazin-1-yl)-1H,3′H-[2,5′-bibenzo[d]imidazol]-2′-yl)phenoxy)propyl)amino)propanoic acid (4:1).

Step 3: N-(2-aminoethyl)-5-(4-(5-(4-methylpiperazin-1-yl)-1H,3′H-[2,5′-bibenzo[d]imidazol]-2′-yl)phen-oxy)pentanamide tetrakis(2,2,2-trifluoroacetate) (101 mg, 0.1 mmol, 1.0 equiv) was added to a solution of 2-(4-(bis(2-chloroethyl)amino)phenoxy)acetic acid (33 mg, 0.11 mmol, 1.1 equiv), HATU (42 mg, 0.11 mmol, 1.1 equiv) and DIPEA (117 mg, 0.9 mmol, 9.0 equiv) in DMF (2 mL). The solution was stirred at RT for 3 h and concentrated in vacuo. The mixture was then purified using reverse phase prep HPLC to afford 2,2,2-trifluoroacetic acid compound with 3-(2-(4-(bis(2-chloroethyl)amino)phenoxy)-N-(3-(4-(5-(4-methylpiperazin-1-yl)-1H,3′H-[2,5′-bibenzo[d]imidazol]-2′-yl)phenoxy)propyl)acetamido)propanoic acid (3:1) (CS0868). MS (M+) 852.9.

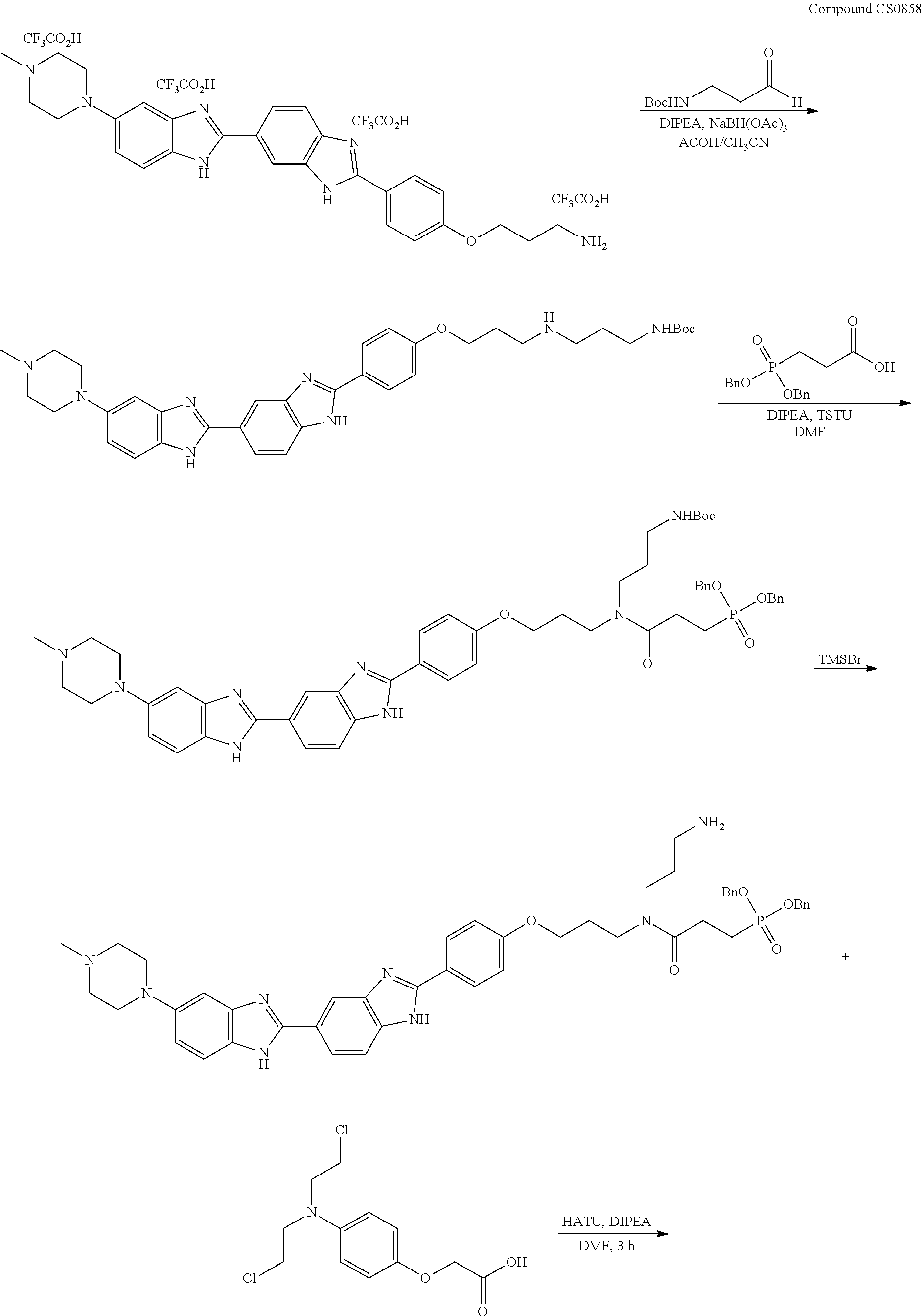

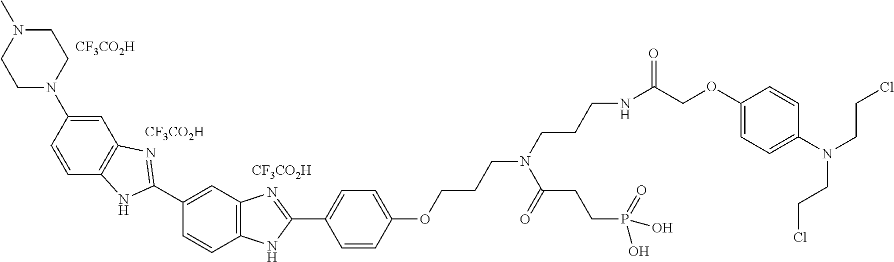 [SMILES: CN1CCN(c2ccc3[nH]c(-c4ccc5[nH]c(-c6ccc(OCCCN(CCCNC(=O)COc7ccc(N(CCCl)CCCl)cc7)C(=O)CCP(=O)(O)O)cc6)nc5c4)nc3c2)CC1 ; "CF3CO2H" ; "CF3CO2H" ; "CF3CO2H"]

Step 1: tert-Butyl (3-oxopropyl)carbamate (17 mg, 0.1 mmol, 1.0 equiv) was mixed with NaBH(OAc)₃ (53 mg, 0.25 mmol, 2.5 equiv) and AcOH (240 μL, 4.0 mmol, 40 equiv) in ACN (2 mL) and stirred for 30 min before a solution of 3-(4-(5-(4-methylpiperazin-1-yl)-1H,3′H-[12,5′-bibenzo[d]imidazol]-2′-yl)phen-oxy)propan-1-amine was added (48 mg, 0.1 mmol, 1.0 equiv). The reaction was stirred at RT for 24 h and purified via reverse phase prep HPLC to afford the intermediate tert-butyl (3-(3-(bis(benzyloxy)phosphoryl)-N-(3-(4-(5-(4-methylpiperazin-1-yl)-1H,1′H-[2,5′-bibenzo[d]imidazol]-2′-yl)phenoxy)propyl)propanamido)propyl)carbamate.

Step 2: 3-(bis(benzyloxy)phosphoryl)propanoic acid (50 mg, 0.15 mmol, 1.5 equiv) was mixed with TSTU (45 mg, 0.15 mmol, 1.5 equiv) and DIPEA (50 μL, 0.3 mmol, 3.0 equiv) in DMF (2 mL). To this solution, tert-butyl (3-(3-(bis(benzyloxy)phosphoryl)-N-(3-(4-(5-(4-methyl-piperazin-1-yl)-1H,1′H-[2,5′-bibenzo-[d]imidazol]-2′-yl)phenoxy)propyl)propanamido)propyl)-c-arbamate (64 mg, 0.1 mmol, 1.0 equiv) was added, and the solution was stirred at RT for 3 h and purified via reverse phase prep HPLC to afford the intermediate tert-butyl (3-(3-(bis(benzyloxy)phosphoryl)-N-(3-(4-(5-(4-methylpiperazin-1-yl)-1H,1′H-[2,5′-bibenzo[d]imi-dazo]-2′-yl)phenoxy)propyl)propanamido)propyl)carbamate.

Step 3: tert-Butyl (3-(3-(bis(benzyloxy)phosphoryl)-N-(3-(4-(5-(4-methylpiperazin-1-yl)-1H,1′H-[2,5′-bibenzo[d]imi-dazo]-2′-yl)phenoxy)propyl)propanamido)propyl)carbamate (95 mg, 0.1 mmol, 1.0 equiv) was dissolved in TMSBr (2 mL) and stirred at RT for 2 h. LC-MS indicated full conversion to the desired product. The reaction was concentrated and purified via reverse phase HPLC purification to afford trifluoroacetate salt of (3-((3-aminopropyl)(3-(4-(5-(4-methylpiperazin-1-yl)-1H,1′H-[2,5′-bibenzo-[d]imidazol]-2′-yl)phenoxy)propyl)amino)-3-oxopro-pyl)phosphonic acid.

Step 4: Trifluoroacetate salt of (3-((3-aminopropyl)(3-(4-(5-(4-methylpiperazin-1-yl)-1H,1′H-[2,5′-bibenzo[d]imidazol]-2′-yl)phenoxy)propyl)amino)-3-oxopro-pyl)phosphonic acid (113 mg, 0.1 mmol, 1.0 equiv) was added to a solution of 2-(4-(bis(2-chloroethyl)amino)phenoxy)acetic acid (33 mg, 0.11 mmol, 1.1 equiv), HATU (42 mg, 0.11 mmol, 1.1 equiv) and DIPEA (117 mg, 0.9 mmol, 9.0 equiv) in DMF (2 mL). The solution was stirred at RT for 3 h and concentrated in vacuo. The mixture was then purified using reverse phase prep HPLC to afford the desired trifluoroacetate salt of (3-((3-(2-(4-(bis(2-chloroethyl)amino)phenoxy)acetamido)propyl)(3-(4-(5-(4-methylpiperazin-1-yl)-1H,1′H-[2,5′-bibenzo-[d]imidazol]-2′-yl)phenoxy)propyl)amino)-3-oxopropyl)phosphonic acid (CS0858). MS (M+) 948.3.

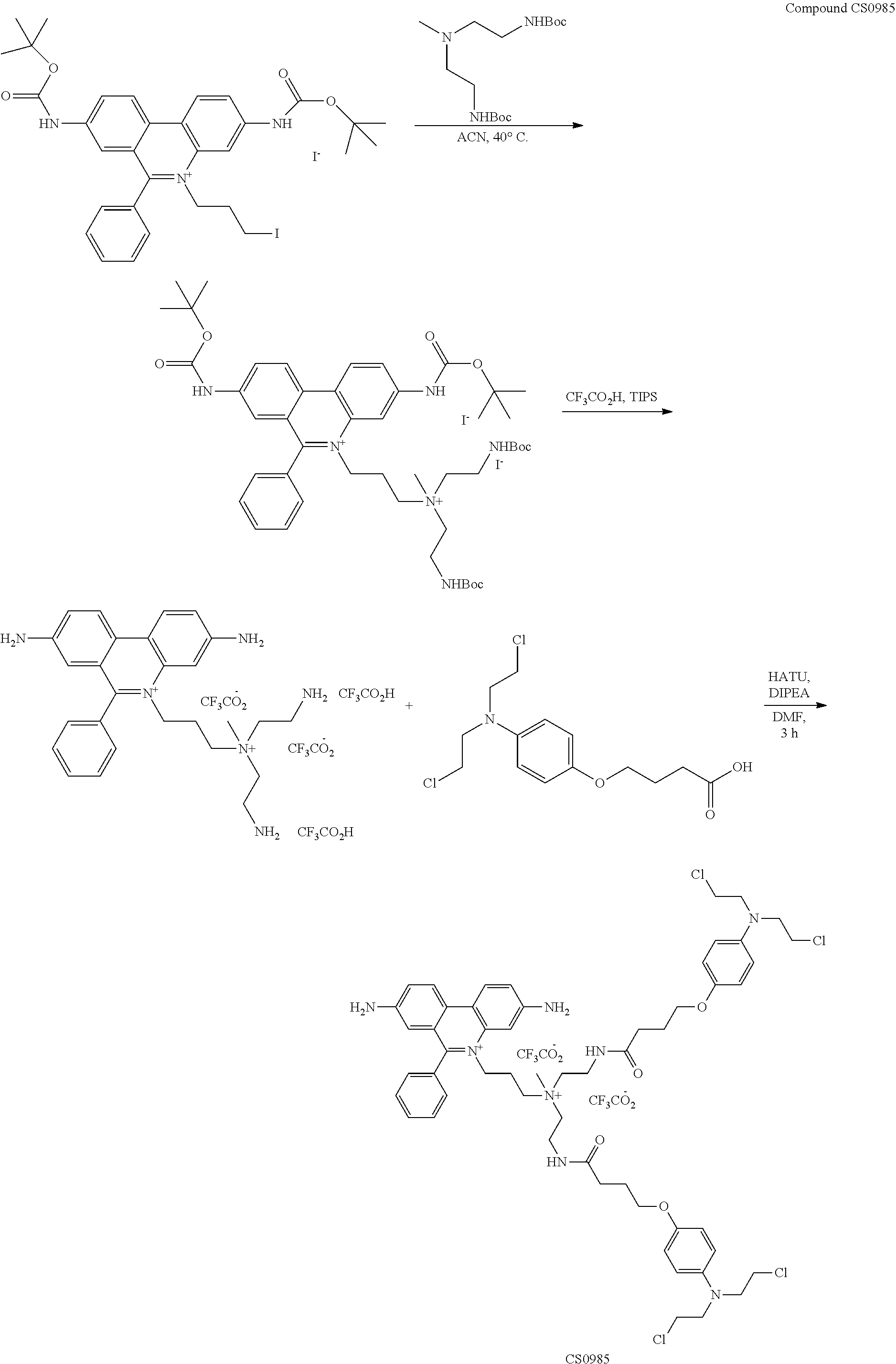

Step 1: 3,8-Bis((tert-butoxycarbonyl)amino)-5-(3-iodopropyl)-6-phenylphenanthridin-5-ium iodide (18 mg, 23 μmol, 1.0 equiv) was mixed with di-tert-butyl ((methylazanediyl)bis(ethane-2,1-diyl))dicarbamate (73 mg, 230 μmol, 10 equiv) in CH₃CN (2 mL) under 40° C. for 6 d. The reaction was concentrated and purified by reverse prep HPLC to afford 5-(3-(bis(2-((tert-butoxycarbonyl)amino)ethyl)(methyl)ammonio)propyl)-3,8-bis((tert-butoxycarbonyl)amino)-6-phenylphenanthridin-5-ium iodide.

Step 2: 5-(3-(Bis(2-((tert-butoxycarbonyl)amino)ethyl)(methyl)ammonio)propyl)-3,8-bis((tert-butoxy-car-bonyl)amino)-6-phenylphenanthridin-5-ium iodide (25 mg, 23 μmol, 1.0 equiv) was dissolved in CF₃CO₂H/TIPS (2 mL/0.2 mL) and stirred at RT for 1 h. The reaction was concentrated and purified by reverse prep HPLC to afford the trifluoroacetate salts of 3,8-diamino-5-(3-(bis(2-aminoethyl)-(methyl)ammonio)propyl)-6-phenylphenanthridin-5-ium.

Step 3: Trifluoroacetate salt of 3,8-diamino-5-(3-(bis(2-aminoethyl)-(methyl)ammonio)propyl)-6-phenylphenanthridin-5-ium (44 mg, 0.1 mmol, 1.0 equiv) was added to a solution of 4-(4-(bis(2-chloroethyl)amino)phenoxy)butanoic acid (76 mg, 0.24 mmol, 2.4 equiv), HATU (91 mg, 0.24 mmol, 2.4 equiv) and DIPEA (117 mg, 0.9 mmol, 9.0 equiv) in DMF (2 mL). The solution was stirred at RT for 3 h and then purified using reverse phase prep HPLC to afford the desired trifluoroacetate salt of 3,8-diamino-5-(3-(bis(2-(4-(4-(bis(2-chloroethyl)amino)phenoxy)butanamido)ethyl)(methyl)ammonio)propyl)-6-phe-nylphenanthridin-5-ium (CS0985). MS (M+)=524.3.

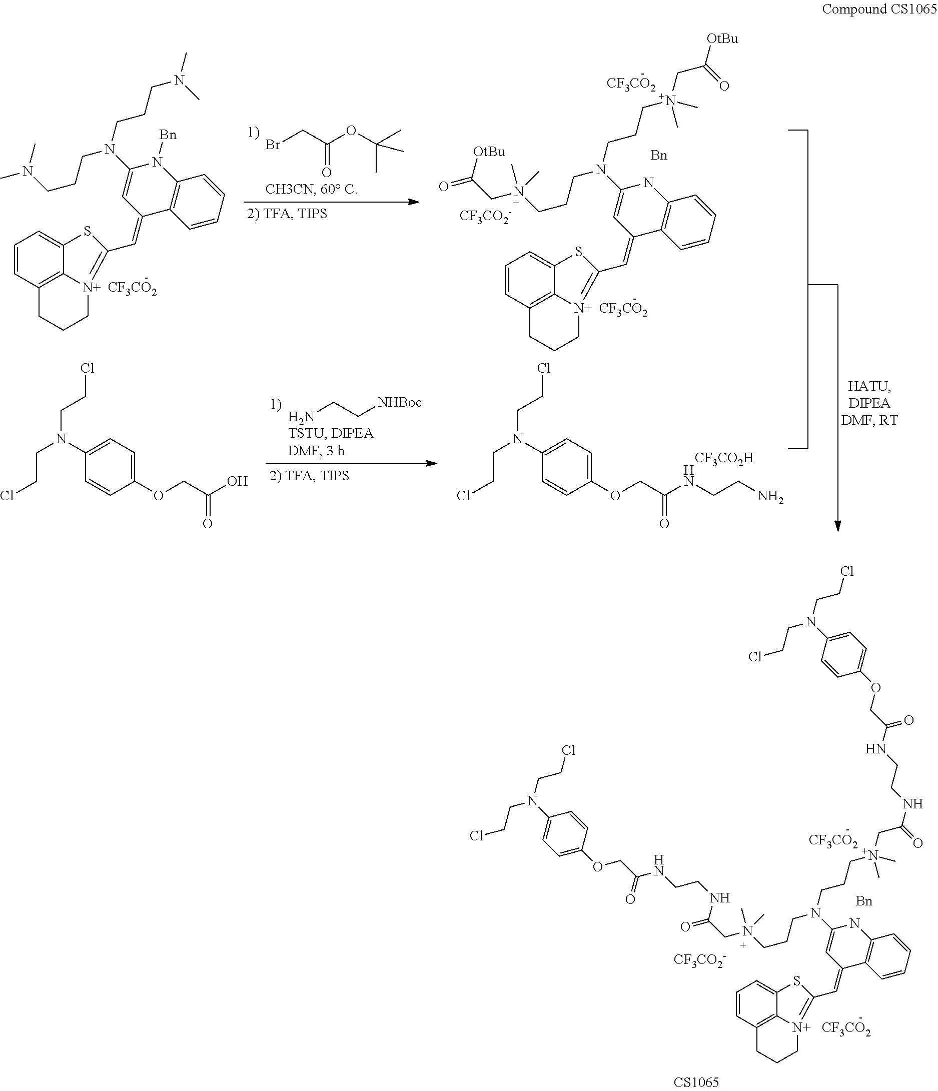

Step 1: (E)-2-((1-benzyl-2-(bis(3-(dimethylamino)propyl)amino)quinolin-4(1H)-ylidene)methyl)-5,6-dihydro-4H-thiazolo[5,4,3-ij]quinolin-3-ium trifluoroacetate (70 mg, 0.1 mmol, 1.0 equiv) was added to a solution of tert-butyl bromoacetate (390 mg, 2 mmol, 20 equiv) in CH₃CN (5 mL), and the reaction was heated to 50° C. for 3 d. The reaction was then concentration and used directly in the next step without further purification.

The intermediate mentioned above (E)-2-((1-benzyl-2-(bis(3-((2-(tert-butoxy)-2-oxoethyl)dimethylam-monio)propyl)amino)quinolin-4(1H)-ylidene)methyl)-5,6-dihydro-4H-thiazolo[5,4,3-ij]quinolin-3-ium trifluoroacetate salt (11.6 mg, 0.01 mmol, 1.0 equiv) was dissolved in TFA/TIPS (2/0.2 mL) and stirred at RT for 2 h. The reaction was concentrated and purified by reverse phase prep HPLC to afford (E)-2-((1-benzyl-2-(bis(3-((carboxymethyl)dimethylammonio)propyl)amino)quinolin-4(1H)-ylidene)methyl)-5,6-dihydro-4H-thiazolo[5,4,3-ij]quinolin-3-ium trifluoroacetate salt.

Step 2: 2-(4-(bis(2-chloroethyl)amino)phenoxy)acetic acid (29 mg, 0.1 mmol, 1.0 equiv) was added to a solution of tert-butyl (2-aminoethyl)carbamate (160 mg, 0.1 mmol, 1.0 equiv), TSTU (33 mg, 0.11 mmol, 1.1 equiv) and DIPEA (26 mg, 0.2 mmol, 2.0 equiv) in CH₃CN (2 mL). The reaction was concentration in vacuo and purified via normal phase silica gel (Heptane/EtOAc) to afford the intermediate tert-butyl (2-(2-(4-(bis(2-chloroethyl)amino)phenoxy)acetamido)ethyl)carbamate, which was subsequently dissolved in TFA/TIPS (2/0.2 mL). The reaction was stirred for 30 min to ensure completion and concentration in vacuo afforded the N-(2-aminoethyl)-2-(4-(bis(2-chloro-ethyl)amino)phenoxy)acetamide, trifluoroacetate salt product which was used in the next step without further purification.

Step 3: N-(2-aminoethyl)-2-(4-(bis(2-chloro-ethyl)amino)phenoxy)acetamide, trifluoroacetate salt (8.8 mg, 0.02 mmol, 2.0 equiv) was added to a solution of (E)-2-((1-benzyl-2-(bis(3-((carboxymethyl)dimethylammonio)propyl)amino)quinolin-4(1H)-ylidene)methyl)-5,6-dihydro-4H-thiazolo[5,4,3-ij]quinolin-3-ium trifluoroacetate salt (10.5 mg, 0.1 mmol, 1.0 equiv), HATU (84 mg, 0.22 mmol, 2.2 equiv) and DIPEA (240 mg, 1.8 mmol, 18.0 equiv) in DMF (2 mL). The solution was stirred at RT for 3 h and concentrated in vacuo. The mixture was then purified using reverse phase prep HPLC to afford (E)-2-((1-benzyl-2-(bis(3-((2-((2-(2-(4-(bis(2-chloroethyl)amino)phenoxy)acetamido)ethyl)amino)-2-oxoethyl)dimethylammonio)propyl)amino)quinolin-4(1H)-ylidene)methyl)-5,6-dihydro-4H-thiazolo[5,4,3-ij]quinolin-3-ium 2,2,2-trifluoroacetate (CS1065). MS (M+)=447.5.

### Example 3

### Viability PCR Experiment with Listeria innocua

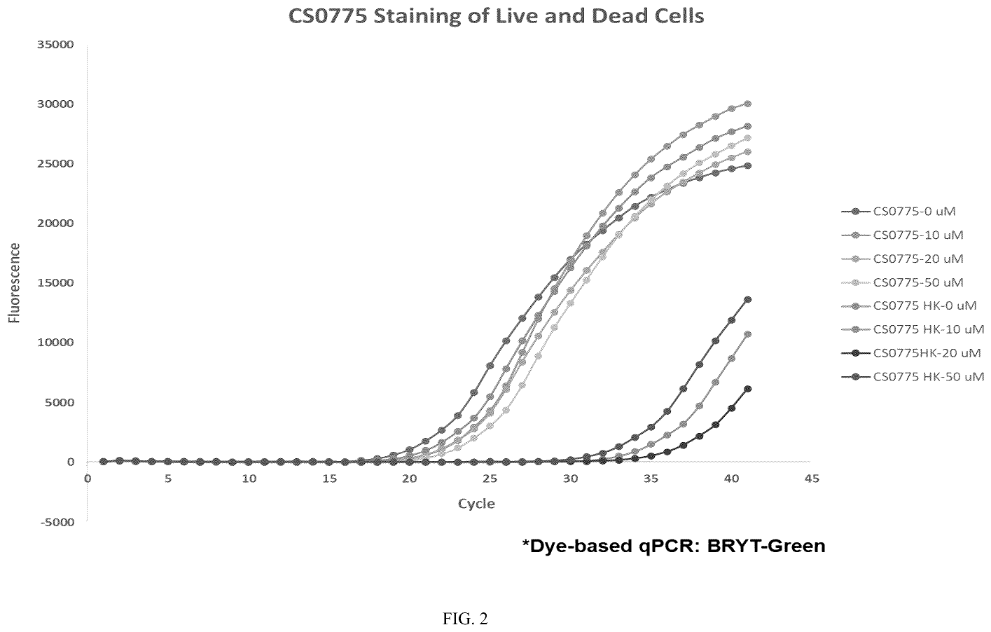

Turbid overnight cultures of Listeria innocua were subcultured into Terrific Broth, grown to early/mid-exponential phase, and concentrated to OD ˜1.0 (˜1×10\hat{ }8 CFU/mL). 200 uL of live or dead (heat-killed for 95° C. for 15 minutes, HK) Listeria innocua cells were incubated with listed concentrations of compound CS0775 at 37° C. for 90 minutes. Inactivation buffer was added to reaction tubes, and reactions incubated at room temperature for 15 minutes. Nucleic acid was purified using the Maxwell automated purification workflow and a dye-based qPCR analysis using primers specific for Listeria innocua was performed. Results are shown in FIG. 2. ΔCt is defined as the qPCR signal threshold difference between dead and live cells. Viable and non-viable cells are clearly distinguished using the described viability PCR workflow.

### Example 4

### Solvent Effect on Permeation in Non-Viable Cells

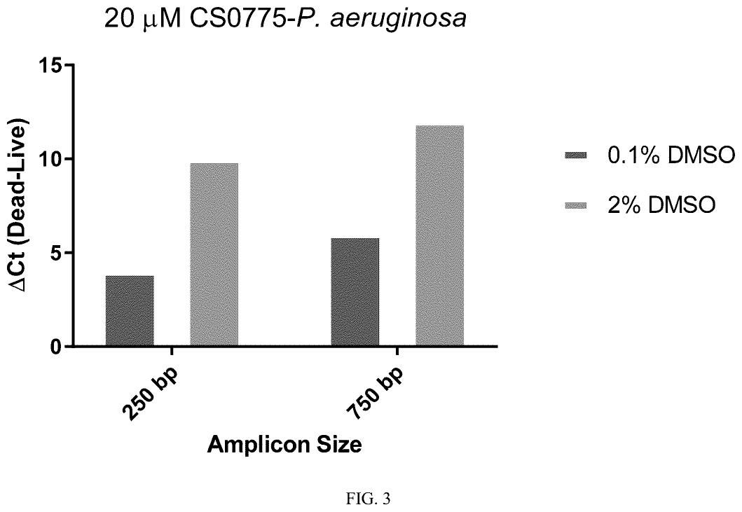

Turbid cultures of Pseudomonas aeruginosa were subcultured into LB Broth, grown to early/mid-exponential phase, and concentrated to OD ˜1.0 (˜1×10\hat{ }8 CFU/mL). 200 uL of live or dead (heat-killed for 95° C. for 15 minutes, HK) Pseudomonas aeruginosa cells were incubated with 20 uM CS0775 at 37° C. for 90 minutes. Inactivation buffer was added to reaction tubes, and reactions incubated at room temperature for 15 minutes. Nucleic acid was purified using the Maxwell automated purification workflow and a dye-based qPCR analysis using primer sets specific for Pseudomonas aeruginosa was performed. Results are shown in FIG. 3. PCR amplicon size notated on horizontal axis. ΔCt is defined as the qPCR signal threshold difference between dead and live cells. Increased concentration of DMSO in the final reaction increases the DNA modification efficiency and subsequent differentiation of non-viable cells from viable cells.

### Example 5

### Amplicon Length Analysis

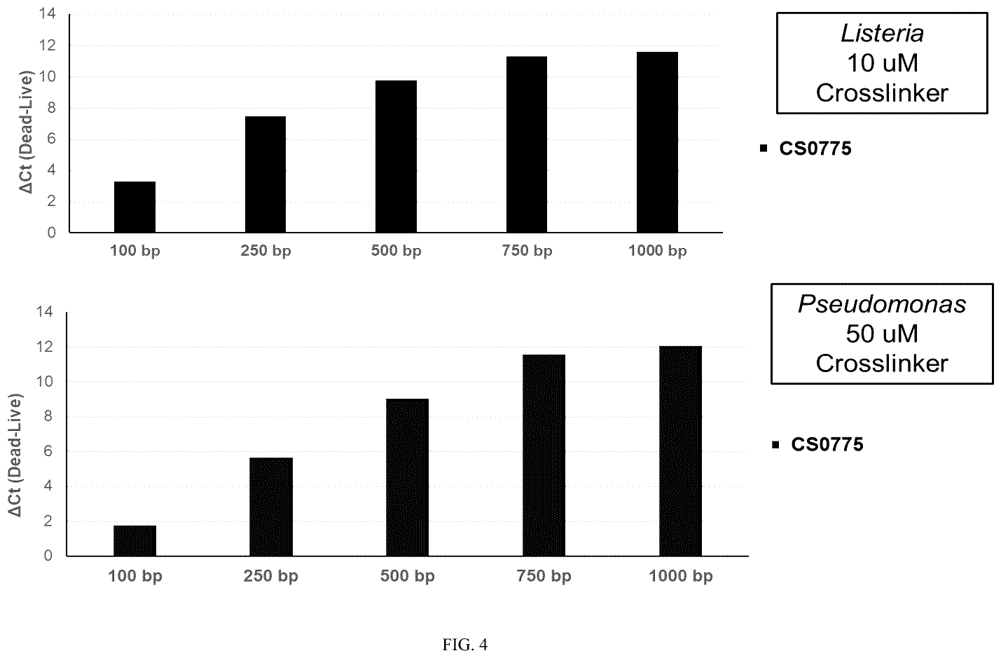

Turbid cultures of Pseudomonas aeruginosa and Listeria innocua were subcultured into LB Broth or Terrific Broth, respectively, grown to early/mid-exponential phase, and concentrated to OD ˜1.0 (˜1×10\hat{ }8 CFU/mL). 200 uL of live or dead (heat-killed for 95° C. for 15 minutes, HK) bacterial cells were incubated with 10 uM (Listeria) or 50 uM (Pseudomonas) CS0775 at 37° C. for 90 minutes. Inactivation buffer was added to reaction tubes, and reactions incubated at room temperature for 15 minutes. Nucleic acid was purified using the Maxwell automated purification workflow and a dye-based qPCR analysis using primers sets specific for Pseudomonas aeruginosa or Listeria innocua was performed. Results are shown in FIG. 4. PCR amplicon size notated on horizontal axis. Live-dead differentiation (ΔCt) increases with amplicon length.

### Example 6

### Effects of Compound Concentration

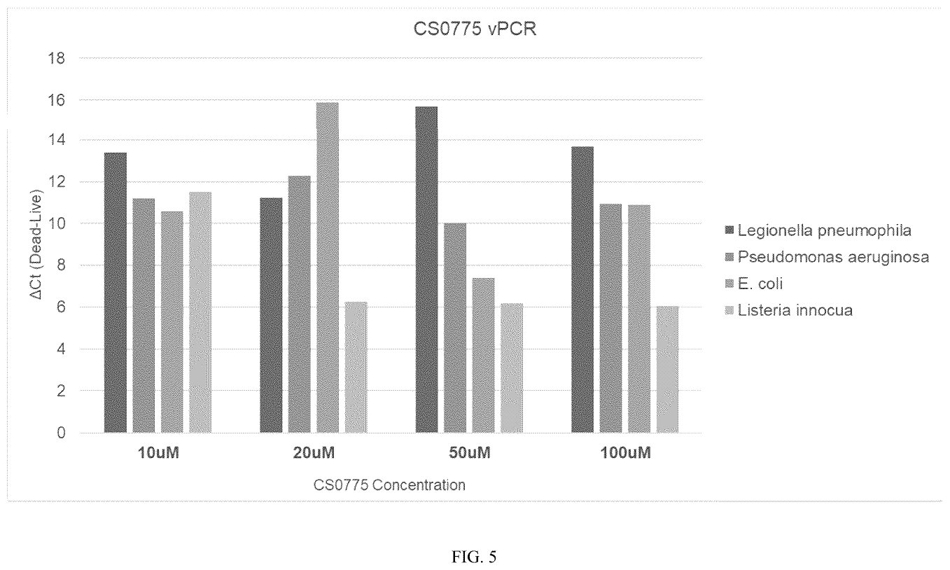

Turbid cultures of Pseudomonas aeruginosa, Escherichia coli, Legionella pneumophila, and Listeria innocua were subcultured into LB Broth (E. coli, P. aeruginosa) Terrific Broth (L. innocua), or Legionella broth (L. pneumophila), grown to early/mid-exponential phase, and concentrated to OD ˜1.0 (˜1×10\hat{ }8 CFU/mL). 200 uL of live or dead (heat-killed for 95° C. for 15 minutes, HK) bacterial cells were incubated with the indicated concentration of CS0775 at 37° C. for 90 minutes. Inactivation buffer was added to reaction tubes, and reactions incubated at room temperature for 15 minutes. Nucleic acid was purified using the Maxwell automated purification workflow and a dye-based qPCR analysis using primers sets specific for the listed bacterial species was performed. Results are shown in FIG. 5. Optimal concentration of compound varies between Gram-negative and Gram-positive bacteria.

### Example 7

### Viability PCR Assays Using Various Compounds

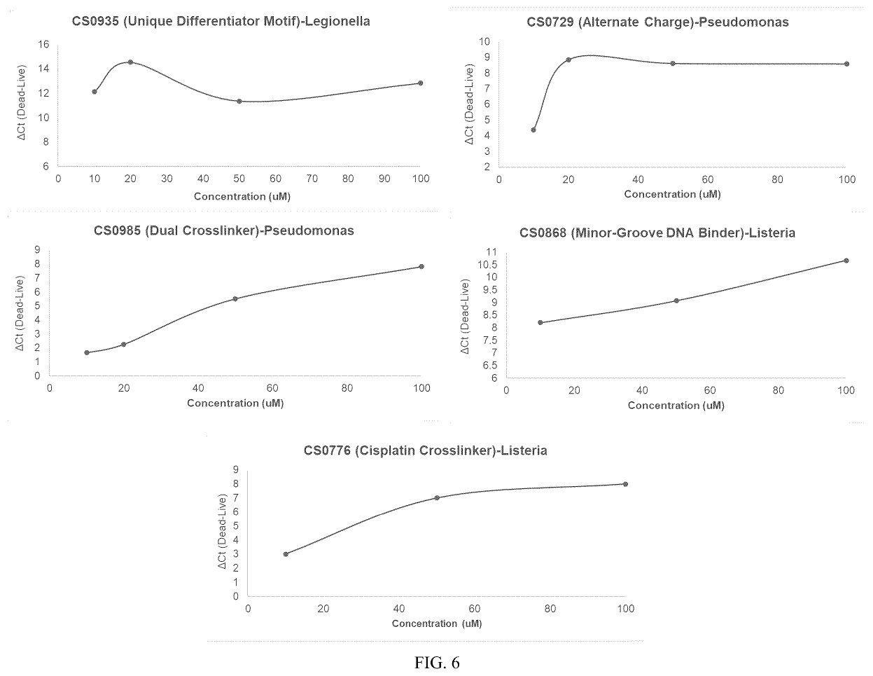

Turbid cultures of Pseudomonas aeruginosa, Legionella pneumophila, and Listeria innocua were subcultured into LB Broth (P. aeruginosa), Terrific Broth (L. innocua), or Legionella broth (L. pneumophila), grown to early/mid-exponential phase, and concentrated to OD ˜1.0 (˜1×10\hat{ }8 CFU/mL). 200 uL of live or dead (heat-killed for 95° C. for 15 minutes, HK) bacterial cells were incubated with the indicated concentration of the indicated compound at 37° C. for 90 minutes. Inactivation buffer was added to reaction tubes, and reactions incubated at room temperature for 15 minutes. Nucleic acid was purified using the Maxwell automated purification workflow and a dye-based qPCR analysis using primers sets specific for the listed bacterial species was performed. Results are shown in FIG. 6. Different classes of compounds can effectively discriminate between viable and non-viable cells when used as part of the viability PCR procedure.

### Example 8

### Assay Sensitivity

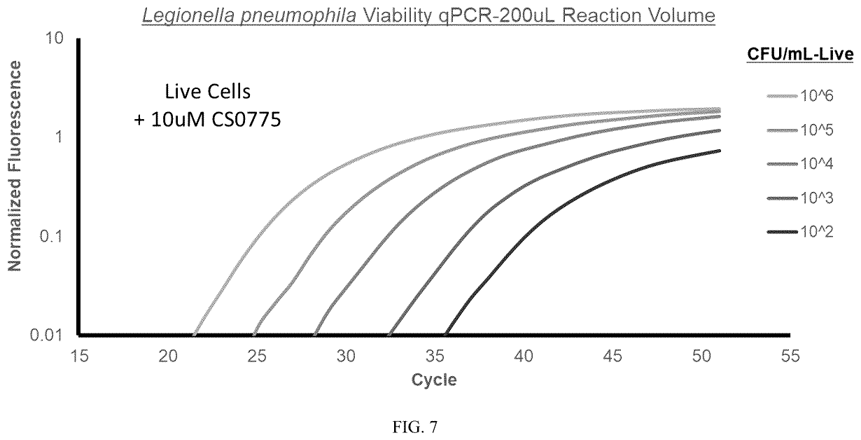

Turbid cultures of Legionella pneumophila were subcultured into Legionella Broth, grown to early/mid-exponential phase, and concentrated to OD ˜1.0 (˜1×10\hat{ }8 CFU/mL). Serial dilutions were performed and 200 uL of the indicated concentration of live Legionella pneumophila cells were incubated with 10 uM CS0775 at 37° C. for 90 minutes. Inactivation buffer was added to reaction tubes, and reactions incubated at room temperature for 15 minutes. Nucleic acid was purified using the Maxwell automated purification workflow and a probe-based qPCR analysis using a primer and probe set specific for Legionella pneumophila was performed. Results are shown in FIG. 7. The assay can detect bacteria at concentrations as low as ˜10² CFU/mL.

### Example 9

### Assays in Samples with Live and Dead Cells

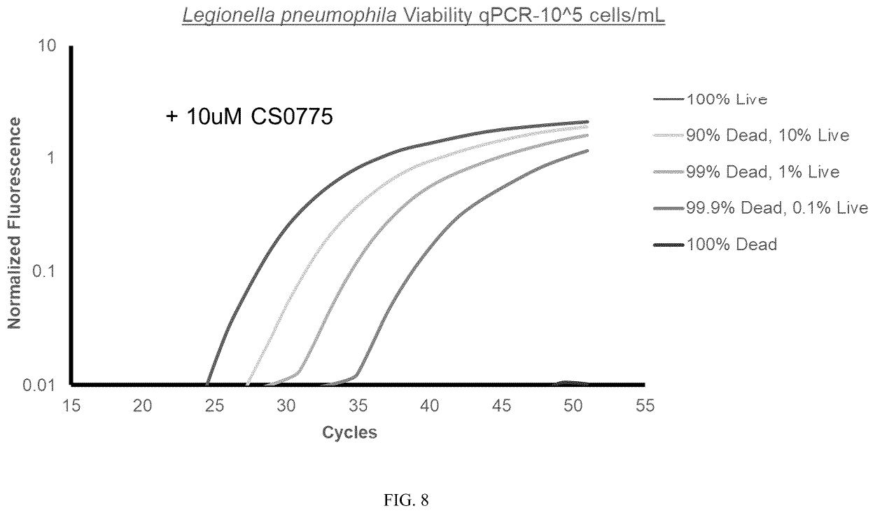

Turbid cultures of Legionella pneumophila were subcultured into Legionella broth, grown to early/mid-exponential phase, and concentrated to OD ˜1.0 (˜1×10\hat{ }8 CFU/mL). Cells were subsequently diluted to 10≡CFU/mL. The listed proportions of live or dead (heat-killed for 95° C. for 15 minutes, HK) bacterial cells were mixed (total volume: 200 uL) and incubated with 10 uM of CS0775 at 37° C. for 90 minutes. Inactivation buffer was added to reaction tubes, and reactions incubated at room temperature for 15 minutes. Nucleic acid was purified using the Maxwell automated purification workflow and a probe-based qPCR analysis using a primer and probe set specific for Legionella pneumophila was performed. Results are shown in FIG. 8. The assay can discriminately detect small proportions of live cells in background of predominantly dead cells.

### Example 10

### Viability PCR Comparison to Traditional Culture

Turbid cultures of Legionella pneumophila were subcultured into Legionella Broth, grown to early/mid-exponential phase, and concentrated to OD ˜1.0 (˜1×10\hat{ }8 CFU/mL). Serial dilutions were performed, plated on Legionella agar to confirm CFU/mL. In parallel, 200 uL of the indicated dilution of live Legionella pneumophila cells were incubated with or without 10 uM CS0775 at 37° C. for 90 minutes. Inactivation buffer was added to reaction tubes, and reactions incubated at room temperature for 15 minutes. Nucleic acid was purified using the Maxwell automated purification workflow and a probe-based qPCR analysis using a primer and probe set specific for Legionella pneumophila was performed. A standard curve of Legionella template DNA was amplified concurrently with the experimental samples to allow for quantification. Results are shown in Table 2. vGU: viable genomic units. The assay produces quantitative data that is comparable to traditional culture-based Legionella pneumophila detection techniques.

TABLE 2
vPCR-Determined Live Cell Numbers

| Culture | | | |
|---|---|---|---|
| Live Cell | Predicted vGU/PCR | qPCR | |
| Concentration | Reaction | −Crosslinker | +Crosslinker |
| 5.2 × 10\hat{ }5 CFU/mL | 2600 | 5181.00 | 2534.00 |
| 5.2 × 10\hat{ }4 CFU/mL | 260 | 537.00 | 211.10 |
| 5.2 × 10\hat{ }3 CFU/mL | 26 | 46.87 | 17.88 |
| 5.2 × 10\hat{ }2 CFU/mL | 2.6 | 4.13 | 1.54 |

### Example 11

### Viability PCR Experiment with Adeno-Associated Virus (AAV)

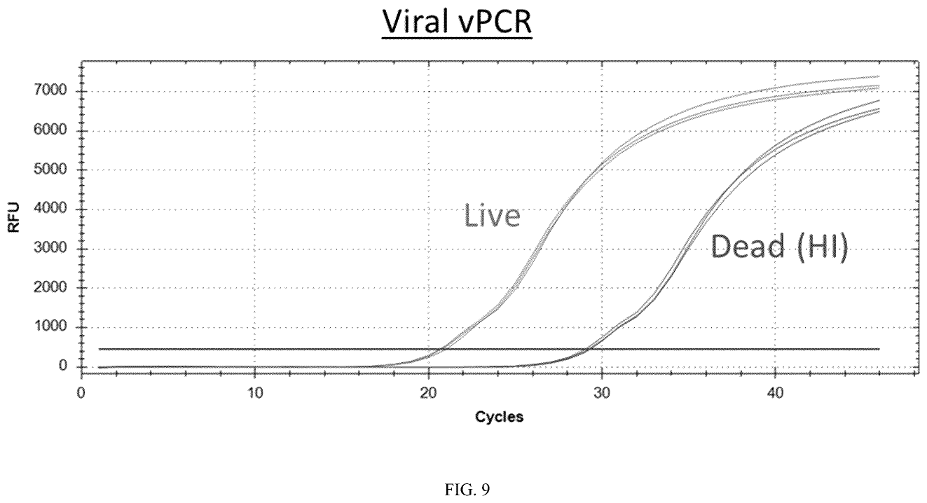

AAV9 reference capsids (Vigene Biosciences; Rockville, MD) containing a recombinant CMV-GFP plasmid were diluted to ˜10\hat{ }8/mL in PBS. 200 uL of live (intact) or dead (heat-inactivated at 75° C. for 10 minutes, HI) AAV9 suspensions were incubated with 1 uM CS0775 at 37° C. for 60 minutes. Inactivation buffer was added to reaction tubes, and reactions incubated at room temperature for 15 minutes. Nucleic acid was purified using the Maxwell automated purification workflow, and a dye-based qPCR analysis using primers specific for GFP was performed. Results are shown in FIG. 9. Intact and heat-inactivated viral capsids are clearly distinguished using the described viability PCR workflow.

### Example 12

### SARS-CoV-2 Viral Infectivity Assay

Three different RT-qPCR assays were designed with different amplicon lengths for use with capsid integrity PCR (Table 3). Regions with higher predicted secondary structure were selected as amplicons.

TABLE 3
SARS-COV-2 RT-qPCR primers and probes

114 bp amplicon

| | |
|---|---|
| E-F1 Fwd | 5′-ACAGGTACGTTAATAGTTAATAGCGT-3′ |
| E-R2 Rev | 5′-ATATTGCAGCAGTACGCACACA-3′ |
| E-P1 | 5′-FAM-ACACTAGCCATCCTTACTGCGCTTCG-3′ |

291 bp amplicon

| | |
|---|---|
| E-F1 Fwd | 5′-ACAGGTACGTTAATAGTTAATAGCGT-3′ |
| E Rev-2 | 5′-AAGCTCTTCAACGGTAATAGTACC-3′ |
| E-P1 | 5′-ACACTAGCCATCCTTACTGCGCTTCG-3′ |

465 bp amplicon

| | |
|---|---|
| N1-Fwd | 5′-GACCCCAAAATCAGCGAAAT-3′ |
| N3 Rev | 5′-TGTAGCACG ATTGCAGCATTG-3′ |
| N1-P | 5′-ACCCCGCATTACGTTTGGTGGACC-3′ |

Nasal swab samples from individuals infected with SARS-CoV-2 in PBS were diluted at 1:100 in PBS. Each dilution was divided into two parts. Samples were treated with different treatment (untreated, 70° C. for 10 minutes, 100° C. for 10 minutes, 70° C. for 10 min in presence of 0.2% CTAB). One was treated with 50 uM CS0775 (viability crosslinker) and the other mock treated (DMSO of same volume). Samples are briefly vortexed and incubated at 37° C. for 60 minutes. Then to the reactions, neutralization solution was added at 1× concentration, mixed well, and incubated for an additional 15 minutes at ambient temperature. This part of the procedure was done in a BSL2 space.

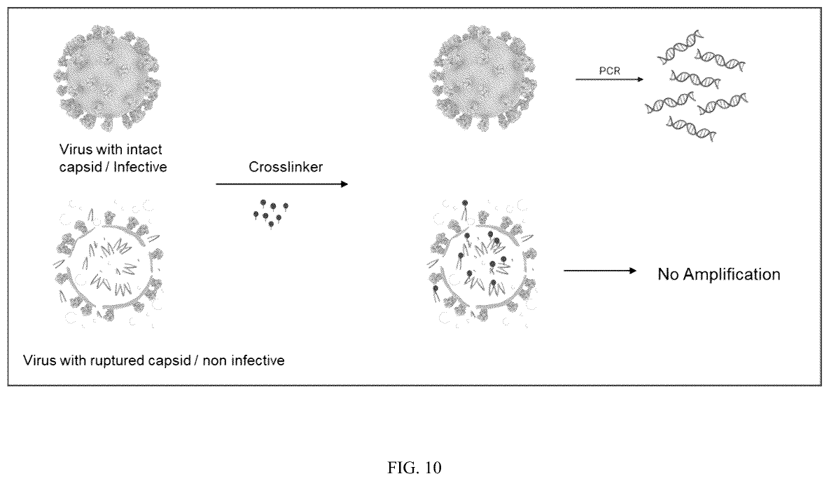

Nucleic acid was extracted using the Maxwell® GMO PureFood Authentication kit. Briefly, to the viral sample, 10 ul of proteinase K and 200 ul of CTAB buffer was added, and the mixture incubated for 30 minutes. This mixture and lysis buffer was added to the well #1 of a Maxwell® cartridge, and nucleic acid was extracted using the Maxwell® automated particle handler. Nucleic acid was extracted in 80 ul of nuclease free water. RT-qPCR was performed using the GoTaq® Enviro RT-qPCR system using N1 Fwd primer (5′-GACCCCAAAATCAGCGAAAT-3′), N1 Probe (5′-FAM-ACCCCGCAT-ZEN-TACGTTTGGTGGACC-IABkFQ-3′) and N3 Rev primer (5′-TGTAGCACGATTGCAGCATTG-3′) in a 20 μL amplification reactions composed of 15 PL reaction mastermix and 5 uL of nucleic acid. 5 μL of nuclease-free water was used as a no-template-control (NTC). RT-qPCR reactions were performed on a BioRad CFX96 Real-Time Thermocycler with the following cycling conditions: reverse transcription for 15 minutes at 45° C., initial denaturation for 2 minutes at 95° C., and 40 cycles of 3 seconds at 95° C. and 30 seconds at 60° C. Ct values for each sample and difference in Ct value between CS0775 treated and untreated samples (DCt) were calculated. The DCt value was directly proportional to the amount of virus with a compromised membrane and thus acted as a surrogate for viral infectivity (FIG. 10).

### Example 13

### AAV Viral Infectivity Assay

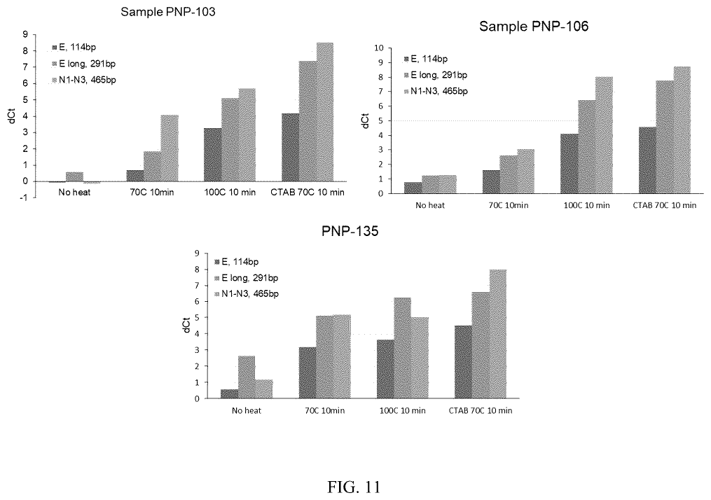

Information on Adeno associated virus (AAV) infectivity can help in determination of dosing for gene therapy applications. AAV does not have a membrane envelope, it has a capsid comprised of three capsid proteins VP1, VP2, and VP3. Experiments were conducted during development of embodiments herein using AAV Reference material (from Vigene Biosciences). The AAV contained a plasmid comprising a GFP insert. First, it was determined whether the multifunctional crosslinker was permeable to AAV-8 particles by treating them with increasing amounts of the multifunctional crosslinker CS0775. It was found that the Ct values are not significantly altered even with 20 uM of the multifunctional crosslinker, indicating that the multifunctional crosslinker is not permeable (FIG. 11A). AAV-8 particles were then heat treated by heating at 75° C. for 10 minutes. A 480-bp amplicon within the GFP was used for analysis (FIG. 11B). For the heat treated AAV-8 sample, the Ct values were significantly right shifted, indicating that the viral particles were compromised, and the multifunctional crosslinker was able to crosslink the DNA genetic material and inhibit the PCR amplification.

A second AAV serotype, AAV9, was also tested (FIG. 11C). AAV-9 was treated with various concentration of the crosslinker (1, 5, 10 and 20 uM, DNase 10U or Dnase+1 uM multifunctional crosslinker). Three different amplicon lengths were analyzed. Compared to Dnase, the crosslinker was able to inhibit free DNA signal.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

1. A multifunctional compound or a salt thereof, the multifunctional compound comprising:
(A) a RNA binding moiety (“RAB moiety”) of formula (I): 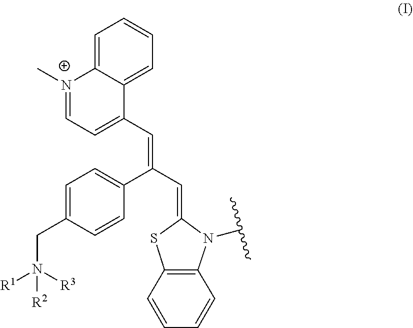
or a tautomer or a salt thereof, wherein:
R¹ and R² are each independently selected from hydrogen, C₁-C₆ alkyl, C₂-C₆ alkenyl, C₂-C₆ alkynyl, aryl, and aryl-C₁-C₆ alkyl, each of which is optionally substituted with 1-3 substituents, or R¹ and R² are taken together with the nitrogen atom to which they are attached to form an optionally substituted heterocyclyl or heteroaryl ring; and
R³ is absent or is C₁-C₆ alkyl;
(B) a live/dead cell differentiating moiety (“LDCD moiety”); and
(C) a nucleic acid modifying moiety (“NAM moiety”) comprising a bischloroethylamine (nitrogen mustard) moiety, a platinum-based moiety, a 1-(chloromethyl)-2,3-dihydro-1H-benzo[e]indolyl moiety, or a pyrrolo[2,1-c][1,4]benzodiazepine (PBD) moiety.

2. (canceled)

3. The multifunctional compound of claim 1, or a salt thereof, in which the multifunctional compound has more than one RAB moiety, more than one LDCD moiety, and/or more than one NAM moiety.

4. (canceled)

5. The multifunctional compound of claim 1, wherein:
(a) R¹ and R² are each independently selected from C₁-C₆ alkyl, and R³ is absent or is C₁-C₆ alkyl;
(b) R¹, R², and R³ are each independently selected from C₁-C₆ alkyl
(c) R³ is absent, and R¹ and R² are taken together with the nitrogen atom to which they are attached to form an optionally substituted monocyclic 5- or 6-membered heterocyclyl or heteroaryl ring having 1, 2, or 3 heteroatoms independently selected from N, O, and S; or
(d) R³ is absent, and R¹ and R² are taken together with the nitrogen atom to which they are attached to form a pyrrolidinyl, piperidinyl, morpholino, piperazinyl, or imidazolyl ring, each of which is optionally substituted with one substituent.

6-9. (canceled)

10. The multifunctional compound of claim 1, wherein the group —NR¹R²R³ in the moiety of formula (I) has a formula selected from:

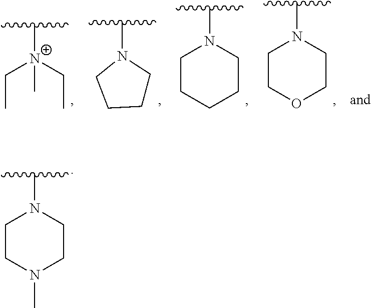

11. The multifunctional compound of claim 1, or a salt thereof, wherein the RAB moiety has a structure selected from:

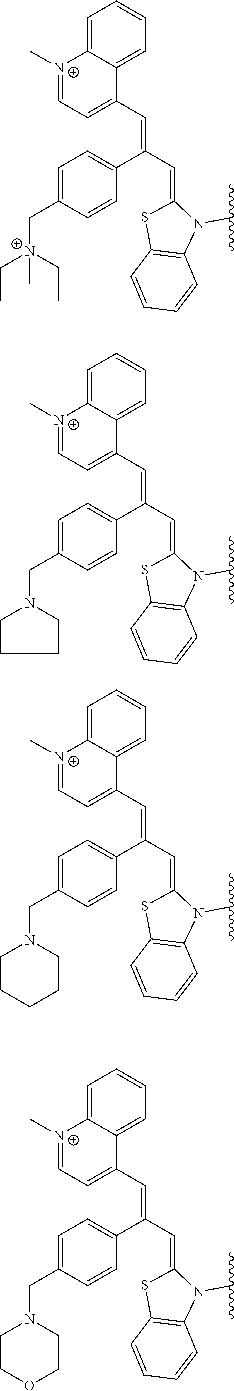 [SMILES: *N1/C(=C/C(=C/c2cc[n+](C)c3ccccc23)c2ccc(C[N+](C)(CC)CC)cc2)Sc2ccccc21 ; *N1/C(=C/C(=C/c2cc[n+](C)c3ccccc23)c2ccc(CN3CCCC3)cc2)Sc2ccccc21 ; *N1/C(=C/C(=C/c2cc[n+](C)c3ccccc23)c2ccc(CN3CCCCC3)cc2)Sc2ccccc21 ; *N1/C(=C/C(=C/c2cc[n+](C)c3ccccc23)c2ccc(CN3CCOCC3)cc2)Sc2ccccc21]

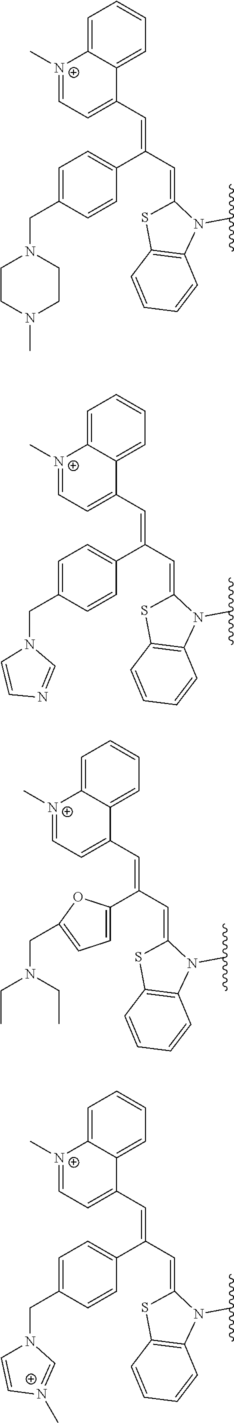 [SMILES: *N1/C(=C/C(=C/c2cc[n+](C)c3ccccc23)c2ccc(CN3CCN(C)CC3)cc2)Sc2ccccc21 ; *N1/C(=C/C(=C/c2cc[n+](C)c3ccccc23)c2ccc(Cn3ccnc3)cc2)Sc2ccccc21 ; *N1/C(=C/C(=C/c2cc[n+](C)c3ccccc23)c2ccc(CN(CC)CC)o2)Sc2ccccc21 ; *N1/C(=C/C(=C/c2cc[n+](C)c3ccccc23)c2ccc(Cn3cc[n+](C)c3)cc2)Sc2ccccc21]

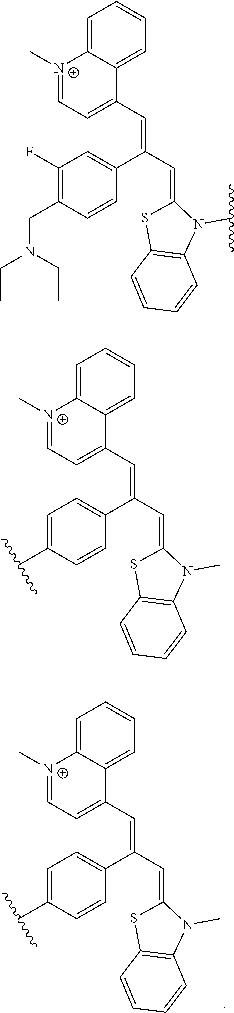

12. (canceled)

13. The multifunctional compound of claim 1, or a salt thereof, wherein the NAM moiety has a structure selected from:

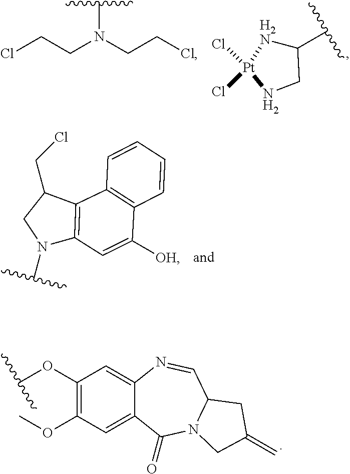

14. (canceled)

15. The multifunctional compound of claim 1, wherein the LDCD moiety comprises at least one quaternary ammonium group.

16. The multifunctional compound of claim 1, or a salt thereof, wherein the LDCD moiety comprises at least one poly(ethylene glycol) moiety.

17. (canceled)

18. The multifunctional compound of claim 1, or a salt thereof, wherein the LDCD moiety comprises a functional group bound to a solid support.

19. The multifunctional compound of claim 1, or a salt thereof, wherein the LDCD moiety comprises a metabolically cleavable group.

20. The multifunctional compound of claim 1, wherein the multifunctional compound is selected from: 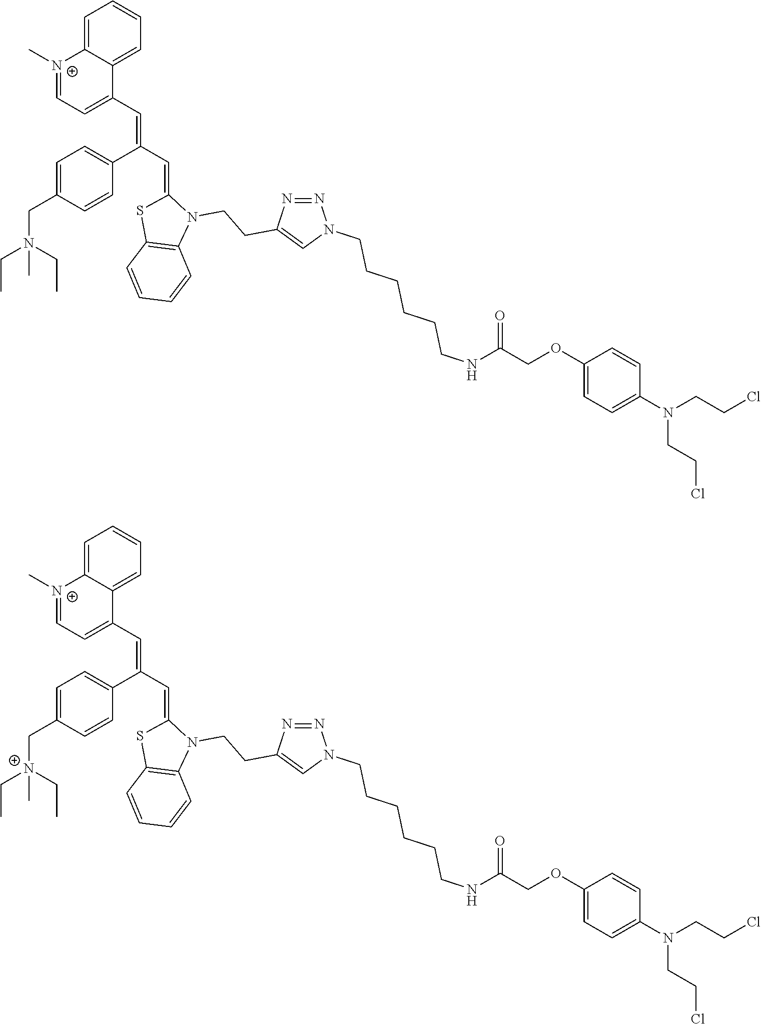 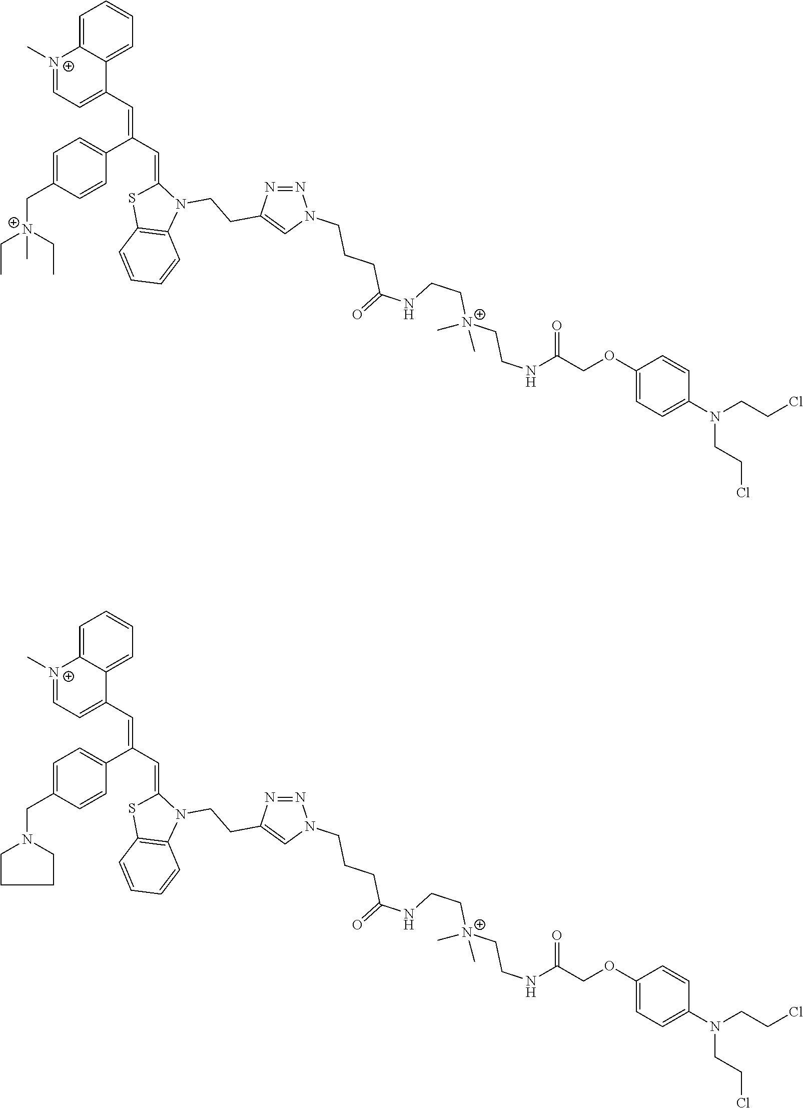 [SMILES: CC[N+](C)(CC)Cc1ccc(C(=C\c2cc[n+](C)c3ccccc23)/C=C2\Sc3ccccc3N2CCc2cn(CCCC(=O)NCC[N+](C)(C)CCNC(=O)COc3ccc(N(CCCl)CCCl)cc3)nn2)cc1 ; C[n+]1ccc(/C=C(/C=C2\Sc3ccccc3N2CCc2cn(CCCC(=O)NCC[N+](C)(C)CCNC(=O)COc3ccc(N(CCCl)CCCl)cc3)nn2)c2ccc(CN3CCCC3)cc2)c2ccccc21] 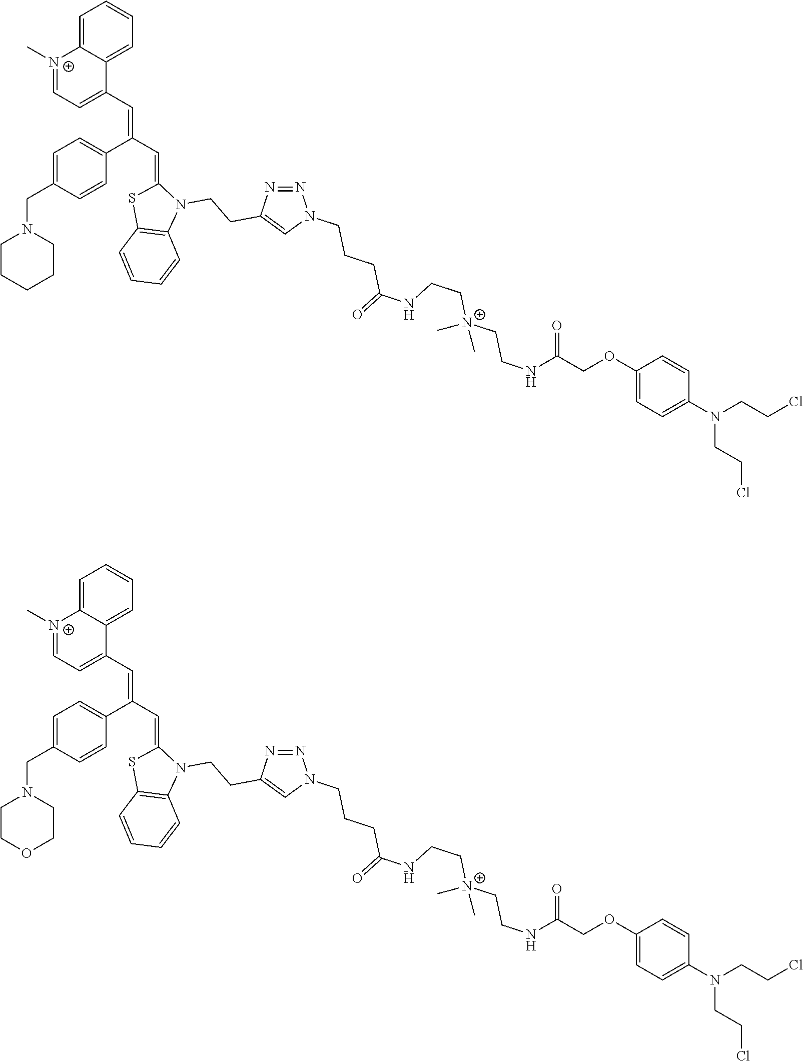 [SMILES: C[n+]1ccc(/C=C(/C=C2\Sc3ccccc3N2CCc2cn(CCCC(=O)NCC[N+](C)(C)CCNC(=O)COc3ccc(N(CCCl)CCCl)cc3)nn2)c2ccc(CN3CCCCC3)cc2)c2ccccc21 ; C[n+]1ccc(/C=C(/C=C2\Sc3ccccc3N2CCc2cn(CCCC(=O)NCC[N+](C)(C)CCNC(=O)COc3ccc(N(CCCl)CCCl)cc3)nn2)c2ccc(CN3CCOCC3)cc2)c2ccccc21] 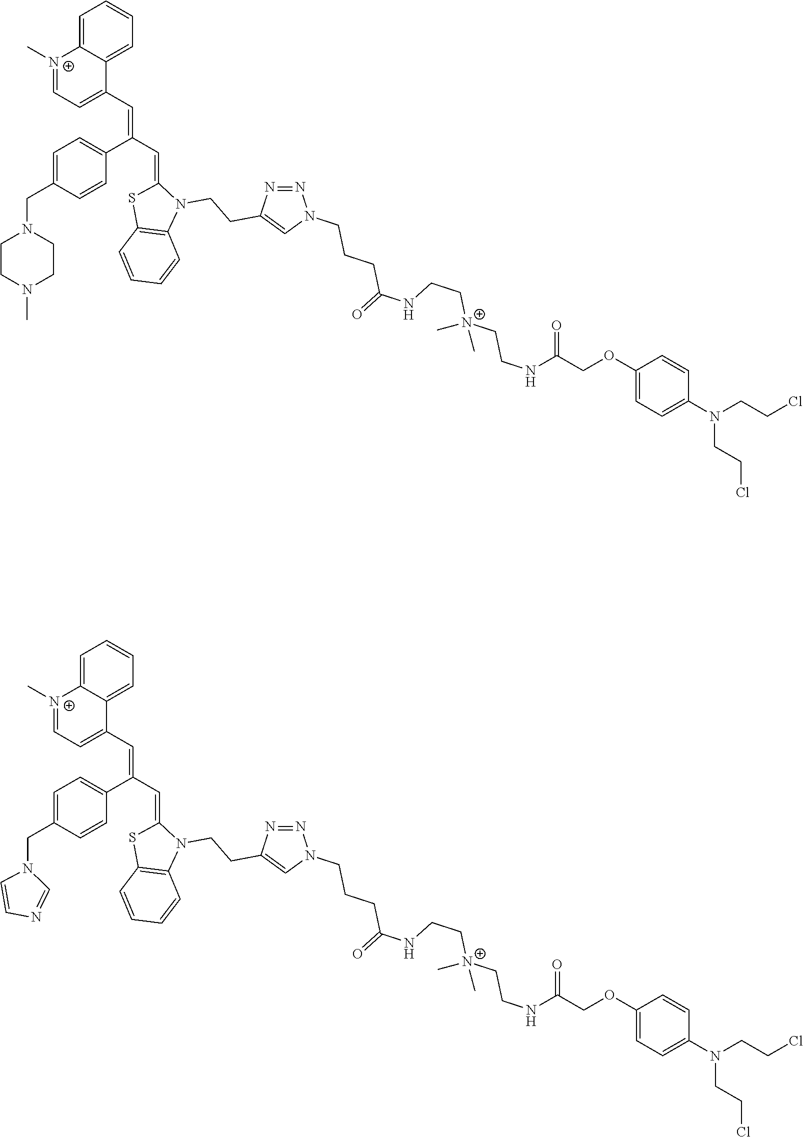 [SMILES: CN1CCN(Cc2ccc(C(=C\c3cc[n+](C)c4ccccc34)/C=C3\Sc4ccccc4N3CCc3cn(CCCC(=O)NCC[N+](C)(C)CCNC(=O)COc4ccc(N(CCCl)CCCl)cc4)nn3)cc2)CC1 ; C[n+]1ccc(/C=C(/C=C2\Sc3ccccc3N2CCc2cn(CCCC(=O)NCC[N+](C)(C)CCNC(=O)COc3ccc(N(CCCl)CCCl)cc3)nn2)c2ccc(Cn3ccnc3)cc2)c2ccccc21] 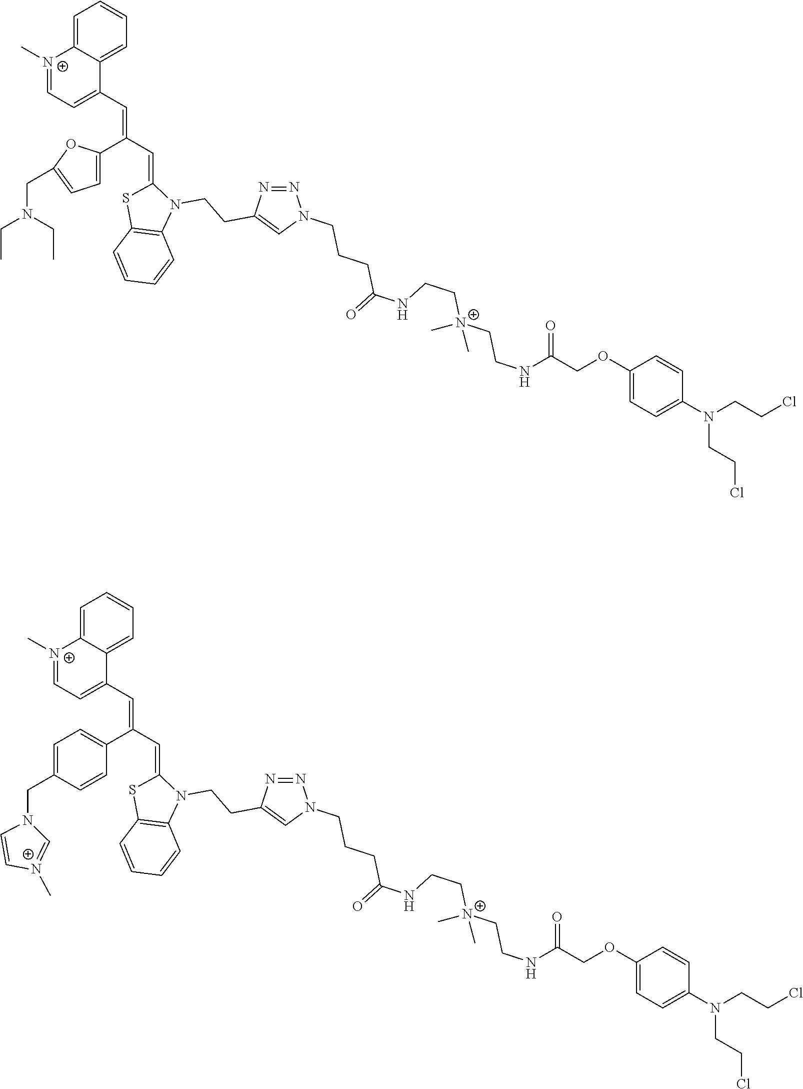 [SMILES: CCN(CC)Cc1ccc(C(=C\c2cc[n+](C)c3ccccc23)/C=C2\Sc3ccccc3N2CCc2cn(CCCC(=O)NCC[N+](C)(C)CCNC(=O)COc3ccc(N(CCCl)CCCl)cc3)nn2)o1 ; C[n+]1ccn(Cc2ccc(C(=C\c3cc[n+](C)c4ccccc34)/C=C3\Sc4ccccc4N3CCc3cn(CCCC(=O)NCC[N+](C)(C)CCNC(=O)COc4ccc(N(CCCl)CCCl)cc4)nn3)cc2)c1] 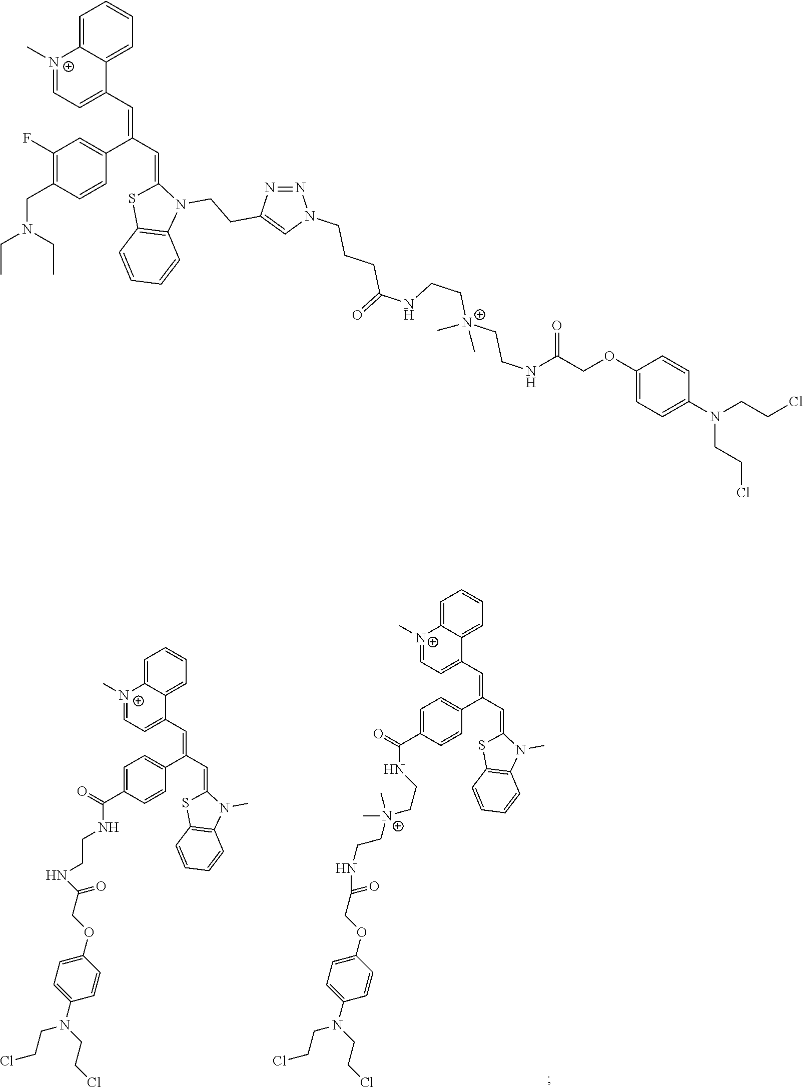 [SMILES: CCN(CC)Cc1ccc(C(=C\c2cc[n+](C)c3ccccc23)/C=C2\Sc3ccccc3N2CCc2cn(CCCC(=O)NCC[N+](C)(C)CCNC(=O)COc3ccc(N(CCCl)CCCl)cc3)nn2)cc1F ; CN1/C(=C/C(=C/c2cc[n+](C)c3ccccc23)c2ccc(C(=O)NCCNC(=O)COc3ccc(N(CCCl)CCCl)cc3)cc2)Sc2ccccc21 ; CN1/C(=C/C(=C/c2cc[n+](C)c3ccccc23)c2ccc(C(=O)NCC[N+](C)(C)CCNC(=O)COc3ccc(N(CCCl)CCCl)cc3)cc2)Sc2ccccc21]
and a salt of any thereof.

21. A method of detecting a viable microorganism or cell in a sample, the method comprising:
(a) contacting the sample with a compound of claim 1, or a salt thereof, to form a first mixture;
(b) contacting the first mixture with an inactivating agent to form a second mixture; and
(c) amplifying nucleic acids from the second mixture to produce a detectable signal, wherein the signal is indicative of the presence of a viable microorganism or cell in the sample.

22-40. (canceled)

41. A method of removing nucleic acids from a sample, the method comprising contacting the sample with a compound of claim 1.

42-50. (canceled)

51. The multifunctional compound of claim 1, or a salt thereof, further comprising:
(D) an affinity or conjugation moiety.

52-74. (canceled)

75. The multifunctional compound of claim 51, or a salt thereof, wherein the LDCD moiety is a branched LDCD moiety.

76. The multifunctional compound of claim 75, wherein the branched LDCD moiety comprises one of the following branch-point functional groups:

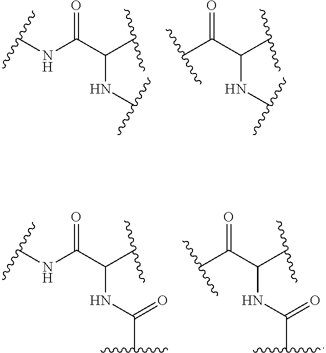 [SMILES: *NC(=O)C(*)N* ; *NC(*)C(*)=O ; *NC(=O)C(*)NC(*)=O ; *C(=O)NC(*)C(*)=O]

77. The multifunctional compound of claim 76, wherein the branched LDCD moiety comprises:

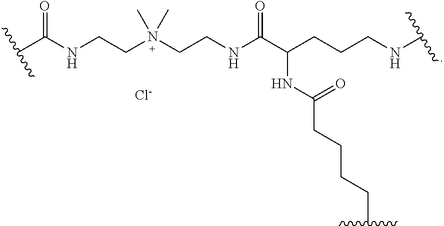

78. The multifunctional compound of claim 51, or a salt thereof, wherein the multifunctional compound comprises an affinity moiety selected from biotin, a peptide tag, and an epitope.

79. (canceled)

80. The multifunctional compound of claim 78, comprising the structure of:

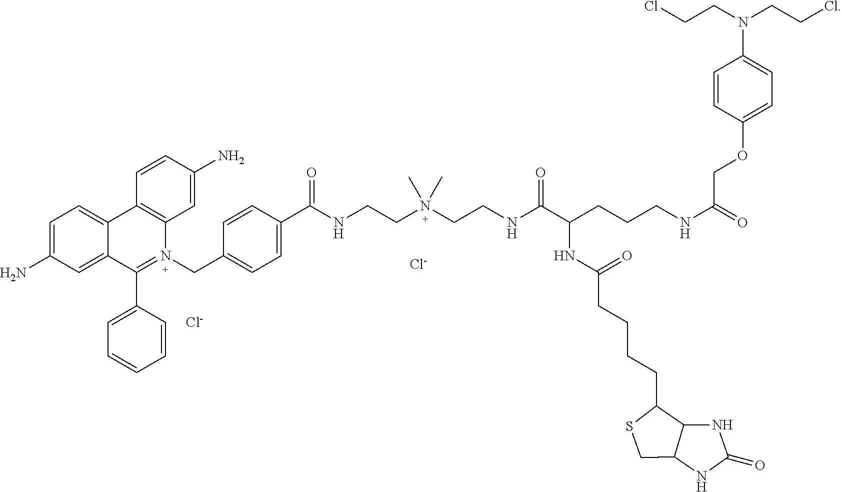

81. The multifunctional compound of claim 51, or a salt thereof, wherein the multifunctional compound comprises a haloalkane conjugation moiety of the structure —(CH₂)ₙ—Y, wherein n is 2, 3, 4, 5, 6, 7, 8, 9, 10, 11, or 12, and Y is F, Cl, Br, or I.

82-87. (canceled)
